(19) United States
(12) Patent Application Publication
(10) Pub. No.: US 2007/0066584 A1
(43) Pub. Date: Mar. 22, 2007

(54) Amido compounds and their use as pharmaceuticals

(76) Inventors: Wenqing Yao, Kennett Square, PA (US); Jincong Zhuo, Boothwyn, PA (US); Ding-Quan Qian, Newark, DE (US)

Correspondence Address: FISH & RICHARDSON PC, P.O. BOX 1022, MINNEAPOLIS, MN, 55440-1022, US

(21) Appl. No.: 11/524,361

(22) Filed: Sep. 20, 2006

Related U.S. Application Data
(60) Provisional application No. 60/719,054, filed on Sep. 21, 2005.
(60) Provisional application No. 60/808,606, filed on May 26, 2006.

(51) Int. Cl. A61K 31/55; A61K 31/496; A61K 31/5377; A61K 31/4545; A61K 31/4747; C07D 413/02; C07D 403/02; C07D 401/02

(52) U.S. Cl. 514/210.2; 514/235.2; 514/278; 514/253.12; 514/217.03; 514/217.04; 514/217.05; 514/326; 540/597; 544/124; 544/360; 546/16; 546/189

(57) ABSTRACT

The present invention relates to inhibitors of 11-β hydroxyl steroid dehydrogenase type 1, antagonists of the mineralocorticoid receptor (MR), and pharmaceutical compositions thereof. The compounds of the invention can be useful in the treatment of various diseases associated with expression or activity of 11-β hydroxyl steroid dehydrogenase type 1 and/or diseases associated with aldosterone excess.

### CROSS REFERENCE TO RELATED APPLICATIONS

This application claims the benefit of U.S. Ser. No. 60/719,054, filed Sep. 21, 2005, and U.S. Ser. No. 60/808,606, filed May 26, 2006, the disclosures of each of which are incorporated herein by reference in their entireties.

### FIELD OF THE INVENTION

The present invention relates to modulators of 11-β hydroxyl steroid dehydrogenase type 1 (11βHSD1) and/or mineralocorticoid receptor (MR), compositions thereof and methods of using the same.

### BACKGROUND OF THE INVENTION

Glucocorticoids are steroid hormones that regulate fat metabolism, function and distribution. In vertebrates, glucocorticoids also have profound and diverse physiological effects on development, neurobiology, inflammation, blood pressure, metabolism and programmed cell death. In humans, the primary endogenously-produced glucocorticoid is cortisol. Cortisol is synthesized in the zona fasciculate of the adrenal cortex under the control of a short-term neuroendocrine feedback circuit called the hypothalamic-pituitary-adrenal (HPA) axis. Adrenal production of cortisol proceeds under the control of adrenocorticotrophic hormone (ACTH), a factor produced and secreted by the anterior pituitary. Production of ACTH in the anterior pituitary is itself highly regulated, driven by corticotropin releasing hormone (CRH) produced by the paraventricular nucleus of the hypothalamus. The HPA axis maintains circulating cortisol concentrations within restricted limits, with forward drive at the diurnal maximum or during periods of stress, and is rapidly attenuated by a negative feedback loop resulting from the ability of cortisol to suppress ACTH production in the anterior pituitary and CRH production in the hypothalamus.

Aldosterone is another hormone produced by the adrenal cortex; aldosterone regulates sodium and potassium homeostasis. Fifty years ago, a role for aldosterone excess in human disease was reported in a description of the syndrome of primary aldosteronism (Conn, (1955), J. Lab. Clin. Med. 45: 6-17). It is now clear that elevated levels of aldosterone are associated with deleterious effects on the heart and kidneys, and are a major contributing factor to morbidity and mortality in both heart failure and hypertension.

Two members of the nuclear hormone receptor superfamily, glucocorticoid receptor (GR) and mineralocorticoid receptor (MR), mediate cortisol function in vivo, while the primary intracellular receptor for aldosterone is the MR. These receptors are also referred to as ‘ligand-dependent transcription factors,’ because their functionality is dependent on the receptor being bound to its ligand (for example, cortisol); upon ligand-binding these receptors directly modulate transcription via DNA-binding zinc finger domains and transcriptional activation domains.

Historically, the major determinants of glucocorticoid action were attributed to three primary factors: 1) circulating levels of glucocorticoid (driven primarily by the HPA axis), 2) protein binding of glucocorticoids in circulation, and 3) intracellular receptor density inside target tissues. Recently, a fourth determinant of glucocorticoid function was identified: tissue-specific pre-receptor metabolism by glucocorticoid-activating and -inactivating enzymes. These 11-beta-hydroxysteroid dehydrogenase (11-β-HSD) enzymes act as pre-receptor control enzymes that modulate activation of the GR and MR by regulation of glucocorticoid hormones. To date, two distinct isozymes of 11-beta-HSD have been cloned and characterized: 11βHSD1 (also known as 11-beta-HSD type 1, 11betaHSD1, HSD11B1, HDL, and HSD11L) and 11βHSD2. 11βHSD1 and 11βHSD2 catalyze the interconversion of hormonally active cortisol (corticosterone in rodents) and inactive cortisone (11-dehydrocorticosterone in rodents). 11βHSD1 is widely distributed in rat and human tissues; expression of the enzyme and corresponding mRNA have been detected in lung, testis, and most abundantly in liver and adipose tissue. 11βHSD1 catalyzes both 11-beta-dehydrogenation and the reverse 11-oxoreduction reaction, although 11βHSD1 acts predominantly as a NADPH-dependent oxoreductase in intact cells and tissues, catalyzing the activation of cortisol from inert cortisone (Low et al. (1994) J. Mol. Endocrin. 13: 167-174) and has been reported to regulate glucocorticoid access to the GR. Conversely, 11βHSD2 expression is found mainly in mineralocorticoid target tissues such as kidney, placenta, colon and salivary gland, acts as an NAD-dependent dehydrogenase catalyzing the inactivation of cortisol to cortisone (Albiston et al. (1994) Mol. Cell. Endocrin. 105: R11-R17), and has been found to protect the MR from glucocorticoid excess, such as high levels of receptor-active cortisol (Blum, et al., (2003) Prog. Nucl. Acid Res. Mol. Biol. 75:173-216).

In vitro, the MR binds cortisol and aldosterone with equal affinity. The tissue specificity of aldosterone activity, however, is conferred by the expression of 11βHSD2 (Funder et al. (1988), Science 242: 583-585). The inactivation of cortisol to cortisone by 11βHSD2 at the site of the MR enables aldosterone to bind to this receptor in vivo. The binding of aldosterone to the MR results in dissociation of the ligand-activated MR from a multiprotein complex containing chaperone proteins, translocation of the MR into the nucleus, and its binding to hormone response elements in regulatory regions of target gene promoters. Within the distal nephron of the kidney, induction of serum and glucocorticoid inducible kinase-1 (sgk-1) expression leads to the absorption of Na⁺ ions and water through the epithelial sodium channel, as well as potassium excretion with subsequent volume expansion and hypertension (Bhargava et al., (2001), Endo 142: 1587-1594).

In humans, elevated aldosterone concentrations are associated with endothelial dysfunction, myocardial infarction, left ventricular atrophy, and death. In attempts to modulate these ill effects, multiple intervention strategies have been adopted to control aldosterone overactivity and attenuate the resultant hypertension and its associated cardiovascular consequences. Inhibition of angiotensin-converting enzyme (ACE) and blockade of the angiotensin type 1 receptor (AT1R) are two strategies that directly impact the rennin-angiotensin-aldosterone system (RAAS). However, although ACE inhibition and AT1R antagonism initially reduce aldosterone concentrations, circulating concentrations of this hormone return to baseline levels with chronic therapy (known as ‘aldosterone escape’). Importantly, co-administration of the MR antagonist Spironolactone or Eplerenone directly blocks the deleterious effects of this escape mechanism and dramatically reduces patient mortality (Pitt et al., New England J. Med. (1999), 341: 709-719; Pitt et al., New England J. Med. (2003), 348: 1309-1321). Therefore, MR antagonism may be an important treatment strategy for many patients with hypertension and cardiovascular disease, particularly those hypertensive patients at risk for target-organ damage.

Mutations in either of the genes encoding the 11-beta-HSD enzymes are associated with human pathology. For example, 11βHSD2 is expressed in aldosterone-sensitive tissues such as the distal nephron, salivary gland, and colonic mucosa where its cortisol dehydrogenase activity serves to protect the intrinsically non-selective MR from illicit occupation by cortisol (Edwards et al. (1988) Lancet 2: 986-989). Individuals with mutations in 11βHSD2 are deficient in this cortisol-inactivation activity and, as a result, present with a syndrome of apparent mineralocorticoid excess (also referred to as ‘SAME’) characterized by hypertension, hypokalemia, and sodium retention (Wilson et al. (1998) Proc. Natl. Acad. Sci. 95: 10200-10205). Likewise, mutations in 11βHSD1, a primary regulator of tissue-specific glucocorticoid bioavailability, and in the gene encoding a co-localized NADPH-generating enzyme, hexose 6-phosphate dehydrogenase (H6PD), can result in cortisone reductase deficiency (CRD), in which activation of cortisone to cortisol does not occur, resulting in adrenocorticotropin-mediated androgen excess. CRD patients excrete virtually all glucocorticoids as cortisone metabolites (tetrahydrocortisone) with low or absent cortisol metabolites (tetrahydrocortisols). When challenged with oral cortisone, CRD patients exhibit abnormally low plasma cortisol concentrations. These individuals present with ACTH-mediated androgen excess (hirsutism, menstrual irregularity, hyperandrogenism), a phenotype resembling polycystic ovary syndrome (PCOS) (Draper et al. (2003) Nat. Genet. 34: 434-439).

The importance of the HPA axis in controlling glucocorticoid excursions is evident from the fact that disruption of homeostasis in the HPA axis by either excess or deficient secretion or action results in Cushing's syndrome or Addison's disease, respectively (Miller and Chrousos (2001) Endocrinology and Metabolism, eds. Felig and Frohman (McGraw-Hill, New York), 4^{th} Ed.: 387-524). Patients with Cushing's syndrome (a rare disease characterized by systemic glucocorticoid excess originating from the adrenal or pituitary tumors) or receiving glucocorticoid therapy develop reversible visceral fat obesity. Interestingly, the phenotype of Cushing's syndrome patients closely resembles that of Reaven's metabolic syndrome (also known as Syndrome X or insulin resistance syndrome) the symptoms of which include visceral obesity, glucose intolerance, insulin resistance, hypertension, type 2 diabetes and hyperlipidemia (Reaven (1993) Ann. Rev. Med. 44: 121-131). However, the role of glucocorticoids in prevalent forms of human obesity has remained obscure because circulating glucocorticoid concentrations are not elevated in the majority of metabolic syndrome patients. In fact, glucocorticoid action on target tissue depends not only on circulating levels but also on intracellular concentration, locally enhanced action of glucocorticoids in adipose tissue and skeletal muscle has been demonstrated in metabolic syndrome. Evidence has accumulated that enzyme activity of 11βHSD1, which regenerates active glucocorticoids from inactive forms and plays a central role in regulating intracellular glucocorticoid concentration, is commonly elevated in fat depots from obese individuals. This suggests a role for local glucocorticoid reactivation in obesity and metabolic syndrome.

Given the ability of 11βHSD1 to regenerate cortisol from inert circulating cortisone, considerable attention has been given to its role in the amplification of glucocorticoid function. 11βHSD1 is expressed in many key GR-rich tissues, including tissues of considerable metabolic importance such as liver, adipose, and skeletal muscle, and, as such, has been postulated to aid in the tissue-specific potentiation of glucocorticoid-mediated antagonism of insulin function. Considering a) the phenotypic similarity between glucocorticoid excess (Cushing's syndrome) and the metabolic syndrome with normal circulating glucocorticoids in the latter, as well as b) the ability of 11βHSD1 to generate active cortisol from inactive cortisone in a tissue-specific manner, it has been suggested that central obesity and the associated metabolic complications in syndrome X result from increased activity of 11βHSD1 within adipose tissue, resulting in ‘Cushing's disease of the omentum’ (Bujalska et al. (1997) Lancet 349: 1210-1213). Indeed, 11βHSD1 has been shown to be upregulated in adipose tissue of obese rodents and humans (Livingstone et al. (2000) Endocrinology 131: 560-563; Rask et al. (2001) J. Clin. Endocrinol. Metab. 86: 1418-1421; Lindsay et al. (2003) J. Clin. Endocrinol. Metab. 88: 2738-2744; Wake et al. (2003) J. Clin. Endocrinol. Metab. 88: 3983-3988).

Additional support for this notion has come from studies in mouse transgenic models. Adipose-specific overexpression of 11βHSD1 under the control of the aP2 promoter in mouse produces a phenotype remarkably reminiscent of human metabolic syndrome (Masuzaki et al. (2001) Science 294: 2166-2170; Masuzaki et al. (2003) J. Clinical Invest. 112: 83-90). Importantly, this phenotype occurs without an increase in total circulating corticosterone, but rather is driven by a local production of corticosterone within the adipose depots. The increased activity of 11βHSD1 in these mice (2-3 fold) is very similar to that observed in human obesity (Rask et al. (2001) J. Clin. Endocrinol. Metab. 86: 1418-1421). This suggests that local 11βHSD1-mediated conversion of inert glucocorticoid to active glucocorticoid can have profound influences whole body insulin sensitivity.

Based on this data, it would be predicted that the loss of 11HSD1 would lead to an increase in insulin sensitivity and glucose tolerance due to a tissue-specific deficiency in active glucocorticoid levels. This is, in fact, the case as shown in studies with 11βHSD1-deficient mice produced by homologous recombination (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). These mice are completely devoid of 11-keto reductase activity, confirming that 11βHSD1 encodes the only activity capable of generating active corticosterone from inert 11-dehydrocorticosterone. 11βHSD1-deficient mice are resistant to diet- and stress-induced hyperglycemia, exhibit attenuated induction of hepatic gluconeogenic enzymes (PEPCK, G6P), show increased insulin sensitivity within adipose, and have an improved lipid profile (decreased triglycerides and increased cardio-protective HDL). Additionally, these animals show resistance to high fat diet-induced obesity. Further, adipose-tissue overexpression of the 11-beta dehydrogenase enzyme, 11bHSD2, which inactivates intracellular corticosterone to 11-dehydrocorticosterone, similarly attenuates weight gain on high fat diet, improves glucose tolerance, and heightens insulin sensitivity. Taken together, these transgenic mouse studies confirm a role for local reactivation of glucocorticoids in controlling hepatic and peripheral insulin sensitivity, and suggest that inhibition of 11βHSD1 activity may prove beneficial in treating a number of glucocorticoid-related disorders, including obesity, insulin resistance, hyperglycemia, and hyperlipidemia.

Data in support of this hypothesis has been published. Recently, it was reported that 11βHSD1 plays a role in the pathogenesis of central obesity and the appearance of the metabolic syndrome in humans. Increased expression of the 11βHSD1 gene is associated with metabolic abnormalities in obese women and that increased expression of this gene is suspected to contribute to the increased local conversion of cortisone to cortisol in adipose tissue of obese individuals (Engeli, et al., (2004) Obes. Res. 12: 9-17).

A new class of 11βHSD1 inhibitors, the arylsulfonamidothiazoles, was shown to improve hepatic insulin sensitivity and reduce blood glucose levels in hyperglycemic strains of mice (Barf et al. (2002) J. Med. Chem. 45: 3813-3815; Alberts et al. Endocrinology (2003) 144: 4755-4762). Additionally, it was recently reported that these selective inhibitors of 11βHSD1 can ameliorate severe hyperglycemia in genetically diabetic obese mice. Data using a structurally distinct series of compounds, the adamantyl triazoles (Hermanowski-Vosatka et al. (2005) J. Exp. Med. 202: 517-527), also indicates efficacy in rodent models of insulin resistance and diabetes, and further illustrates efficacy in a mouse model of atherosclerosis, perhaps suggesting local effects of corticosterone in the rodent vessel wall. Thus, 11βHSD1 is a promising pharmaceutical target for the treatment of the Metabolic Syndrome (Masuzaki, et al., (2003) Curr. Drug Targets Immune Endocr. Metabol. Disord. 3: 255-62).

A. Obesity and Metabolic Syndrome

As described above, multiple lines of evidence suggest that inhibition of 11βHSD1 activity can be effective in combating obesity and/or aspects of the metabolic syndrome cluster, including glucose intolerance, insulin resistance, hyperglycemia, hypertension, hyperlipidemia, and/or atherosclerosis/coronary heart disease. Glucocorticoids are known antagonists of insulin action, and reductions in local glucocorticoid levels by inhibition of intracellular cortisone to cortisol conversion should increase hepatic and/or peripheral insulin sensitivity and potentially reduce visceral adiposity. As described above, 11βHSD1 knockout mice are resistant to hyperglycemia, exhibit attenuated induction of key hepatic gluconeogenic enzymes, show markedly increased insulin sensitivity within adipose, and have an improved lipid profile. Additionally, these animals show resistance to high fat diet-induced obesity (Kotelevstev et al. (1997) Proc. Natl. Acad. Sci. 94: 14924-14929; Morton et al. (2001) J. Biol. Chem. 276: 41293-41300; Morton et al. (2004) Diabetes 53: 931-938). In vivo pharmacology studies with multiple chemical scaffolds have confirmed the critical role for 11HSD1 in regulating insulin resistance, glucose intolerance, dyslipidemia, hypertension, and atherosclerosis. Thus, inhibition of 11βHSD1 is predicted to have multiple beneficial effects in the liver, adipose, skeletal muscle, and heart, particularly related to alleviation of component(s) of the metabolic syndrome, obesity, and/or coronary heart disease.

B. Pancreatic Function

Glucocorticoids are known to inhibit the glucose-stimulated secretion of insulin from pancreatic beta-cells (Billaudel and Sutter (1979) Horm. Metab. Res. 11: 555-560). In both Cushing's syndrome and diabetic Zucker fa/fa rats, glucose-stimulated insulin secretion is markedly reduced (Ogawa et al. (1992) J. Clin. Invest. 90: 497-504). 11βHSD1 mRNA and activity has been reported in the pancreatic islet cells of ob/ob mice and inhibition of this activity with carbenoxolone, an 11βHSD1 inhibitor, improves glucose-stimulated insulin release (Davani et al. (2000) J. Biol. Chem. 275: 34841-34844). Thus, inhibition of 11βHSD1 is predicted to have beneficial effects on the pancreas, including the enhancement of glucose-stimulated insulin release and the potential for attenuating pancreatic beta-cell decompensation.

C. Cognition and Dementia

Mild cognitive impairment is a common feature of aging that may be ultimately related to the progression of dementia. In both aged animals and humans, inter-individual differences in general cognitive function have been linked to variability in the long-term exposure to glucocorticoids (Lupien et al. (1998) Nat. Neurosci. 1: 69-73). Further, dysregulation of the HPA axis resulting in chronic exposure to glucocorticoid excess in certain brain subregions has been proposed to contribute to the decline of cognitive function (McEwen and Sapolsky (1995) Curr. Opin. Neurobiol. 5: 205-216). 11βHSD1 is abundant in the brain, and is expressed in multiple subregions including the hippocampus, frontal cortex, and cerebellum (Sandeep et al. (2004) Proc. Natl. Acad. Sci. Early Edition: 1-6). Treatment of primary hippocampal cells with the 11βHSD1 inhibitor carbenoxolone protects the cells from glucocorticoid-mediated exacerbation of excitatory amino acid neurotoxicity (Rajan et al. (1996) J. Neurosci. 16: 65-70). Additionally, 11βHSD1-deficient mice are protected from glucocorticoid-associated hippocampal dysfunction that is associated with aging (Yau et al. (2001) Proc. Natl. Acad. Sci. 98: 4716-4721). In two randomized, double-blind, placebo-controlled crossover studies, administration of carbenoxolone improved verbal fluency and verbal memory (Sandeep et al. (2004) Proc. Natl. Acad. Sci. Early Edition: 1-6). Thus, inhibition of 11βHSD1 is predicted to reduce exposure to glucocorticoids in the brain and protect against deleterious glucocorticoid effects on neuronal function, including cognitive impairment, dementia, and/or depression.

D. Intra-Ocular Pressure

Glucocorticoids can be used topically and systemically for a wide range of conditions in clinical ophthalmology. One particular complication with these treatment regimens is corticosteroid-induced glaucoma. This pathology is characterized by a significant increase in intra-ocular pressure (IOP). In its most advanced and untreated form, IOP can lead to partial visual field loss and eventually blindness. IOP is produced by the relationship between aqueous humour production and drainage. Aqueous humour production occurs in the non-pigmented epithelial cells (NPE) and its drainage is through the cells of the trabecular meshwork. 11βHSD1 has been localized to NPE cells (Stokes et al. (2000) Invest. Ophthalmol. Vis. Sci. 41: 1629-1683; Rauz et al. (2001) Invest. Ophthalmol. Vis. Sci. 42: 2037-2042) and its function is likely relevant to the amplification of glucocorticoid activity within these cells. This notion has been confirmed by the observation that free cortisol concentration greatly exceeds that of cortisone in the aqueous humour (14:1 ratio). The functional significance of 11βHSD1 in the eye has been evaluated using the inhibitor carbenoxolone in healthy volunteers (Rauz et al. (2001) Invest. Ophthalmol. Vis. Sci. 42: 2037-2042). After seven days of carbenoxolone treatment, IOP was reduced by 18%. Thus, inhibition of 11βHSD1 in the eye is predicted to reduce local glucocorticoid concentrations and IOP, producing beneficial effects in the management of glaucoma and other visual disorders.

E. Hypertension

Adipocyte-derived hypertensive substances such as leptin and angiotensinogen have been proposed to be involved in the pathogenesis of obesity-related hypertension (Matsuzawa et al. (1999) Ann. N.Y. Acad. Sci. 892: 146-154; Wajchenberg (2000) Endocr. Rev. 21: 697-738). Leptin, which is secreted in excess in aP2-11βHSD1 transgenic mice (Masuzaki et al. (2003) J. Clinical Invest. 112: 83-90), can activate various sympathetic nervous system pathways, including those that regulate blood pressure (Matsuzawa et al. (1999) Ann. N.Y. Acad. Sci. 892: 146-154). Additionally, the renin-angiotensin system (RAS) has been shown to be a major determinant of blood pressure (Walker et al. (1979) Hypertension 1: 287-291). Angiotensinogen, which is produced in liver and adipose tissue, is the key substrate for renin and drives RAS activation. Plasma angiotensinogen levels are markedly elevated in aP2-11βHSD1 transgenic mice, as are angiotensin II and aldosterone (Masuzaki et al. (2003) J. Clinical Invest. 112: 83-90). These forces likely drive the elevated blood pressure observed in aP2-11βHSD1 transgenic mice. Treatment of these mice with low doses of an angiotensin II receptor antagonist abolishes this hypertension (Masuzaki et al. (2003) J. Clinical Invest. 112: 83-90). This data illustrates the importance of local glucocorticoid reactivation in adipose tissue and liver, and suggests that hypertension may be caused or exacerbated by 11βHSD1 activity. Thus, inhibition of 11βHSD1 and reduction in adipose and/or hepatic glucocorticoid levels is predicted to have beneficial effects on hypertension and hypertension-related cardiovascular disorders.

F. Bone Disease

Glucocorticoids can have adverse effects on skeletal tissues. Continued exposure to even moderate glucocorticoid doses can result in osteoporosis (Cannalis (1996) J. Clin. Endocrinol. Metab. 81: 3441-3447) and increased risk for fractures. Experiments in vitro confirm the deleterious effects of glucocorticoids on both bone-resorbing cells (also known as osteoclasts) and bone forming cells (osteoblasts). 11βHSD1 has been shown to be present in cultures of human primary osteoblasts as well as cells from adult bone, likely a mixture of osteoclasts and osteoblasts (Cooper et al. (2000) Bone 27: 375-381), and the 11βHSD1 inhibitor carbenoxolone has been shown to attenuate the negative effects of glucocorticoids on bone nodule formation (Bellows et al. (1998) Bone 23: 119-125). Thus, inhibition of 11βHSD1 is predicted to decrease the local glucocorticoid concentration within osteoblasts and osteoclasts, producing beneficial effects in various forms of bone disease, including osteoporosis.

Small molecule inhibitors of 11βHSD1 are currently being developed to treat or prevent 11βHSD1-related diseases such as those described above. For example, certain amide-based inhibitors are reported in WO 2004/089470, WO 2004/089896, WO 2004/056745, and WO 2004/065351. Antagonists of 11βHSD1 have been evaluated in human clinical trials (Kurukulasuriya, et al., (2003) Curr. Med. Chem. 10: 123-53).

In light of the experimental data indicating a role for 11βHSD1 in glucocorticoid-related disorders, metabolic syndrome, hypertension, obesity, insulin resistance, hyperglycemia, hyperlipidemia, type 2 diabetes, atherosclerosis, androgen excess (hirsutism, menstrual irregularity, hyperandrogenism), polycystic ovary syndrome (PCOS), and other diseases, therapeutic agents aimed at augmentation or suppression of these metabolic pathways, by modulating glucocorticoid signal transduction at the level of 11βHSD1 are desirable.

Furthermore, because the MR binds to aldosterone (its natural ligand) and cortisol with equal affinities, compounds that are designed to interact with the active site of 11βHSD1 (which binds to cortisone/cortisol) may also interact with the MR and act as antagonists. Because the MR is implicated in heart failure, hypertension, and related pathologies including atherosclerosis, arteriosclerosis, coronary artery disease, thrombosis, angina, peripheral vascular disease, vascular wall damage, and stroke, MR antagonists are desirable and may also be useful in treating complex cardiovascular, renal, and inflammatory pathologies including disorders of lipid metabolism including dyslipidemia or hyperlipoproteinaemia, diabetic dyslipidemia, mixed dyslipidemia, hypercholesterolemia, hypertriglyceridemia, as well as those associated with type 1 diabetes, type 2 diabetes, obesity, metabolic syndrome, and insulin resistance, and general aldosterone-related target-organ damage.

As evidenced herein, there is a continuing need for new and improved drugs that target 11βHSD1. The compounds, compositions and methods described herein help meet this and other needs.

### SUMMARY OF THE INVENTION

The present invention provides, inter alia, compounds of Formula I: 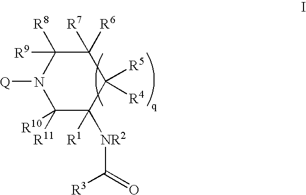
or pharmaceutically acceptable salts or prodrugs thereof, wherein constituent members are defined herein.

The present invention further provides compositions comprising compounds of the invention and a pharmaceutically acceptable carrier.

The present invention further provides methods of modulating 11βHSD1 or MR by contacting 11βHSD1 or MR with a compound of the invention.

The present invention further provides methods of inhibiting 11βHSD1 or MR by contacting 11βHSD1 or MR with a compound of the invention.

The present invention further provides methods of treating diseases associated with activity or expression of 11βHSD1 or MR.

### DETAILED DESCRIPTION

The present invention provides, inter alia, compounds of Formula I: 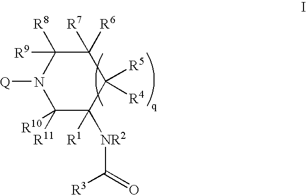
or pharmaceutically acceptable salt or prodrug thereof, wherein:

Q is —SO₂-Cy, —C(O)O-Cy or —C(O)NR^{A}R^{B};

Cy is cycloalkyl or heterocycloalkyl, each optionally substituted by 1, 2, 3, 4 or 5 —W—X—Y-Z;

R^{A} and R^{B} are independently selected from H, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl, and heterocycloalkylalkyl, wherein said C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl, or heterocycloalkylalkyl are each optionally substituted with 1, 2, 3, 4 or 5 —W—X—Y-Z;

or R^{A} and R^{B} together with the N atom to which they are attached form a 4-20 membered heterocycloalkyl ring optionally substituted with 1, 2, 3, 4 or 5 —W—X—Y-Z;

R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;

R² is H, C₁₋₆ alkyl, arylalkyl, heteroarylalkyl, cycloalkyl, cycloalkylalkyl, heterocycloalkyl or heterocycloalkylalkyl, wherein said C₁₋₆ alkyl, arylalkyl, heteroarylalkyl, cycloalkyl, cycloalkylalkyl, heterocycloalkyl or heterocycloalkylalkyl is optionally substituted by 1, 2 or 3 R¹⁴;

R³ is H, NR^{3a}R^{3b}, C₁₋₆ alkyl, aryl, cycloalkyl, heteroaryl, or heterocycloalkyl, wherein said C₁₋₆ alkyl, aryl, cycloalkyl, heteroaryl, or heterocycloalkyl is optionally substituted by 1, 2 or 3 —W′—X′—Y′-Z′;

R^{3a} and R^{3b} are independently selected from H, C₁₋₆ alkyl, aryl, cycloalkyl, heteroaryl, and heterocycloalkyl, wherein said C₁₋₆ alkyl, aryl, cycloalkyl, heteroaryl, or heterocycloalkyl is optionally substituted by 1, 2 or 3 —W′—X′—Y′-Z′;

or R^{3a} and R^{3b} together with the N atom to which they are attached form a 4-14 membered heterocycloalkyl group which is optionally substituted by 1, 2 or 3 —W′—X′—Y′-Z′;

R⁴, R⁵, R⁶, R⁷, R⁸, R⁹, R¹⁰ and R¹¹ are independently selected from H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl is optionally substituted by 1, 2 or 3 R¹⁴;

or R² and R³ together with the nitrogen and carbon atoms to which they are attached form a 3-14 membered heterocycloalkyl group which is optionally substituted by 1, 2 or 3 R¹⁴;

or R⁴ and R⁵ together with the carbon atom to which they are attached form a 3-14 membered cycloalkyl or 3-14 membered heterocycloalkyl group which is optionally substituted by 1, 2 or 3 R¹⁴;

or R⁶ and R⁷ together with the carbon atom to which they are attached form a 3-14 membered cycloalkyl or 3-14 membered heterocycloalkyl group which is optionally substituted by 1, 2 or 3 R¹⁴;

or R⁸ and R⁹ together with the carbon atom to which they are attached form a 3-14 membered cycloalkyl or 3-14 membered heterocycloalkyl group which is optionally substituted by 1, 2 or 3 R¹⁴;

or R¹⁰ and R¹¹ together with the carbon atom to which they are attached form a 3-14 membered cycloalkyl or 3-14 membered heterocycloalkyl group which is optionally substituted by 1, 2 or 3 R¹⁴;

or R⁴ and R⁶ together with the carbon atoms to which they are attached form a 3-7 membered fused cycloalkyl group or 3-7 membered fused heterocycloalkyl group which is optionally substituted by 1, 2 or 3 R¹⁴;

or R⁶ and R⁸ together with the carbon atoms to which they are attached form a 3-7 membered fused cycloalkyl group or 3-7 membered fused heterocycloalkyl group which is optionally substituted by 1, 2 or 3 R¹⁴;

R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};

W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of said C₁₋₆ alkylenyl, C₂₋₆ alkenylenyl, or C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino, and C₂₋₈ dialkylamino;

X, X′ and X″ are independently selected from absent, C₁₋₆ alkylenyl, C₂₋₆ alkenylenyl, C₂₋₆ alkynylenyl, aryl, cycloalkyl, heteroaryl, and heterocycloalkyl, wherein each of said C₁₋₆ alkylenyl, C₂₋₆ alkenylenyl, C₂₋₆ alkynylenyl, cycloalkyl, heteroaryl, and heterocycloalkyl is optionally substituted by one or more substituents independently selected from halo, CN, NO₂, OH, C₁₋₄ alkoxy, C₁₋₄ haloalkoxy, amino, C₁₋₄ alkylamino, and C₂₋₈ dialkylamino;

Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein each of said C₁₋₆ alkylenyl, C₂₋₆ alkenylenyl, and C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino, and C₂₋₈ dialkylamino;

Z, Z′ and Z″ are independently selected from H, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, pentahalosulfanyl, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl, and heterocycloalkyl, wherein each of said C₁₋₆ alkyl, C₂₋₆ alkenyl, C₂₋₆ alkynyl, aryl, cycloalkyl, heteroaryl or heterocycloalkyl is optionally substituted by 1, 2 or 3 substituents independently selected from halo, C₁₋₆ alkyl, C₂₋₆ alkenyl, C₂₋₆ alkynyl, C₁₋₄ haloalkyl, aryl, cycloalkyl, heteroaryl, heterocycloalkyl, pentahalosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, and S(O)₂NR^{c}R^{d};

wherein two —W—X—Y-Z attached to the same atom optionally form a 3-14 membered cycloalkyl or 3-14 membered heterocycloalkyl group optionally substituted by 1, 2 or 3 —W″—X″—Y″-Z″;

wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;

wherein —W—X—Y-Z is other than H;

wherein —W′—X′—Y′-Z′ is other than H;

wherein —W″—X″—Y″-Z″ is other than H;

R^{a} and R^{a′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, aryl, arylalkyl, heteroaryl, heteroarylalkyl, cycloalkyl or heterocycloalkyl;

R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c} and R^{d} together with the N atom to which they are attached form a 4-, 5-, 6- or 7-membered heterocycloalkyl group;

R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{c′} and R^{d′} together with the N atom to which they are attached form a 4-, 5-, 6- or 7-membered heterocycloalkyl group;

R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{e} and R^{f} together with the N atom to which they are attached form a 4-, 5-, 6- or 7-membered heterocycloalkyl group; and

q is 1 or 2.

In some embodiments, when q is 1 and R⁴ is H, then R⁵ is other than —NHC(O)R^{g}, wherein R^{g} is heteroaryl substituted by halo.

In some embodiments, when Q is —C(O)NR^{A}R^{B} and R^{A} is H, C₁₋₄ alkyl, or arylalkyl substituted by halo, then R^{B} is other than C₁₋₄ alkyl optionally substituted by COOH, COO(C₁₋₄ alkyl), aryl substituted by halo, or aryloxy substituted by 1 or 2 C₁₋₆ alkyl.

In some embodiments, R³ is other than piperidin-3-yl which is N-substituted by Q¹, wherein: Q¹ is -Cy¹, —SO₂-Cy¹, —C(O)Cy¹, —C(O)O-Cy¹, or C(O)NR^{h}Cy¹; Cy¹ is aryl, heteroaryl, cycloalkyl, or heterocycloalkyl, each optionally substituted by 1, 2, 3, 4 or 5 —W—X—Y-Z; and R^{h} is H, C₁₋₆ alkyl, aryl, heteroaryl, C₃₋₇ cycloalkyl, heterocycloalkyl, arylalkyl, heteroarylalkyl, (C₃₋₇ cycloalkyl)alkyl, or heterocycloalkylalkyl.

In some embodiments, R³ is other than N-substituted piperidin-3-yl.

In some embodiments, Q is —SO₂-Cy.

In some embodiments, Q is —C(O)O-Cy.

In some embodiments, Q is —C(O)NR^{A}R^{B}.

In some embodiments:

Q is —C(O)NR^{A}R^{B};

R^{A} is H, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein each of said C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl is optionally substituted by 1, 2, 3, 4 or 5 —W—X—Y-Z; and

R^{B} is cycloalkyl, heterocycloalkyl, cycloalkylalkyl or heterocycloalkylalkyl, each optionally substituted by 1, 2, 3, 4 or 5 —W—X—Y-Z.

In some embodiments:

Q is —C(O)NR^{A}R^{B};

R^{A} is H, C₁₋₁₀ alkyl, C₂₋₁₀ alkenyl, C₂₋₁₀ alkynyl, cycloalkyl, heterocycloalkyl, cycloalkylalkyl or heterocycloalkylalkyl, wherein each of said C₁₋₁₀ alkyl, C₂₋₁₀ alkenyl, C₂₋₁₀ alkynyl, cycloalkyl, heterocycloalkyl, cycloalkylalkyl or heterocycloalkylalkyl is optionally substituted by 1, 2, 3, 4 or 5 —W—X—Y-Z; and

R^{B} is cycloalkyl or heterocycloalkyl, each optionally substituted by 1, 2, 3, 4 or 5 —W—X—Y-Z.

In some embodiments Q is —C(O)NR^{A}R^{B} and R^{A} and R^{B} together with the N atom to which they are attached form a 4-20 membered heterocycloalkyl ring optionally substituted by 1, 2, 3, 4 or 5 —W—X—Y-Z.

In some embodiments, Q is —C(O)NR^{A}R^{B} and R^{A} and R^{B} together with the N atom to which they are attached form a moiety having the formula: 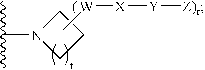
wherein:

r is 0, 1, 2, 3, 4 or 5; and

t is 1, 2, 3, 4, or 5.

In some embodiments, Q is —C(O)NR^{A}R^{B} and R^{A} and R^{B} together with the N atom to which they are attached form a moiety having the formula: 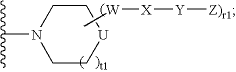
wherein:

r1 is 0, 1, 2 or 3;

t1 is 0 or 1; and

U is CH₂, NH or O.

In some embodiments, Q is —C(O)NR^{A}R^{B} and R^{A} and R^{B} together with the N atom to which they are attached form a moiety having the formula: 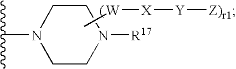
wherein:

r1 is 0, 1, 2 or 3;

R¹⁷ is C(O)R^{b}, C(O)NR^{c}R^{d}, C(O)OR^{a}, C₁₋₆ alkyl, aryl or heteroaryl, wherein each of said C₁₋₆ alkyl, aryl or heteroaryl is optionally substituted by 1, 2 or 3, halo, C₁₋₄ alkyl, C₁₋₄ alkoxy or C₁₋₄ haloalkyl.

In some embodiments, Q is —C(O)NR^{A}R^{B} and R^{A} and R^{B} together with the N atom to which they are attached form a moiety having the formula: 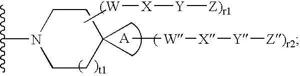
wherein:

ring A is a 3-14 membered cycloalkyl group or a 3-14 membered heterocycloalkyl group;

r1 is 0, 1, 2 or 3; and

r2 is 0, 1, 2, or 3.

In some further embodiments, ring A is a 5-10 membered heterocycloalkyl group. In yet further embodiments, ring-forming carbon atoms and heteroatoms of a heterocycloalkyl group of ring A are optionally substituted by oxo.

In some embodiments, Q is —C(O)NR^{A}R^{B} and R^{A} and R^{B} together with the N atom to which they are attached form a moiety having Formula IIa or IIb: 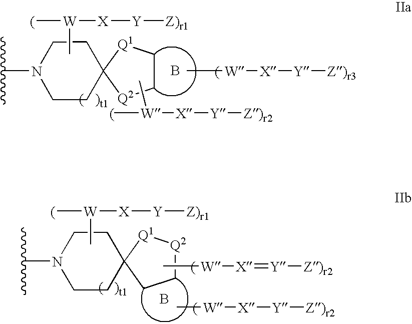
wherein:

Q¹ is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;

Q² is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;

ring B is a fused 5- or 6-membered aryl or fused 5- or 6-membered heteroaryl group;

r1 is 0, 1 or 2;

r2 is 0, 1 or 2;

r3 is 0, 1, or 2; and

the sum of r1, r2 and r3 is 0, 1, 2 or 3.

In some embodiments, Q is —C(O)NR^{A}R^{B} and R^{A} and R^{B} together with the N atom to which they are attached form pyrrolidinyl, piperidinyl, piperizinyl, morpholino, 1,2,3,6-tetrahydro-pyridinyl, 3-oxo-piperazinyl, azepanyl or azocanyl, each optionally substituted by 1, 2 or 3 OH, CN, C₁₋₄ alkyl, C₁₋₄ alkoxy, arylalkyl, heterocycloalkyl, aryl, heteroaryl, NR^{c}C(O)R^{d}, NR^{c}C(O)OR^{a}, C(O)R^{b}, C(O)NR^{c}R^{d} or C(O)OR^{a}, wherein each of said aryl or heteroaryl is optionally substituted by 1, 2 or 3 halo, CN, C₁₋₄ alkyl, C₁₋₄ alkoxy or C₁₋₄ haloalkyl.

In some embodiments, Cy is cycloalkyl optionally substituted by 1, 2, 3, 4 or 5 —W—X—Y-Z.

In some embodiments, Cy is heterocycloalkyl optionally substituted by 1, 2, 3, 4 or 5 —W—X—Y-Z;

In some embodiments, R² is H.

In some embodiments, R³ is cycloalkyl or heterocycloalkyl, each optionally substituted by 1, 2 or 3 —W′—X′—Y′-Z′.

In some embodiments, R³ is cycloalkyl or heterocycloalkyl, each optionally substituted by OH.

In some embodiments, R³ is adamantyl optionally substituted by 1, 2 or 3 —W′—X′—Y′-Z′.

In some embodiments, R³ is adamantyl optionally substituted by OH.

In some embodiments, R³ is NR^{3a}R^{3b}; and R^{3a} and R^{3b} together with the N atom to which they are attached form a 4-14 membered heterocycloalkyl group which is optionally substituted by 1, 2 or 3 —W′—X′—Y′-Z′.

In some embodiments, R³ is 8-azabicyclo[3.2.1]octanyl optionally substituted by 1, 2 or 3 —W′—X′—Y′-Z′.

In some embodiments, R³ is 8-azabicyclo[3.2.1]octanyl optionally substituted by OH.

In some embodiments, R⁴, R⁵, R⁶, R⁷, R⁸, R⁹, R¹⁰ and R¹¹ are each H.

In some embodiments, R¹ is H.

In some embodiments, R² is H.

In some embodiments, the compounds of the invention have Formula III: 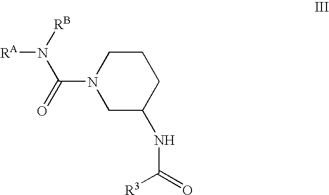
wherein R^{A} and R^{B} together with the N atom to which they are attached form a 4-20 membered heterocycloalkyl ring which is optionally substituted by 1, 2, 3, 4 or 5 —W—X—Y-Z.

In some embodiments, the compounds of the invention have Formula III and R³ is cycloalkyl or heterocycloalkyl, each optionally substituted by 1, 2 or 3 —W′—X′—Y′-Z′.

In some embodiments, the compounds of the invention have Formula IV: 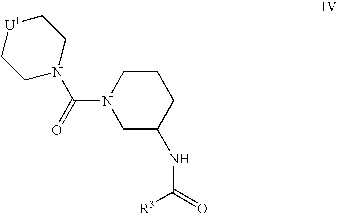
wherein:

U¹ is O, NR¹⁷ or CR¹⁸R¹⁹;

R¹⁷ is C(O)R^{b}, C(O)NR^{c}R^{d}, C(O)OR^{a}, C₁₋₆ alkyl, aryl or heteroaryl, wherein each of said C₁₋₆ alkyl, aryl or heteroaryl is optionally substituted by 1, 2 or 3, halo, C₁₋₄ alkyl, C₁₋₄ alkoxy or C₁₋₄ haloalkyl;

R¹⁸ is H, OH, CN, C₁₋₄ alkyl, C₁₋₄ alkoxy, arylalkyl, heterocycloalkyl, aryl or heteroaryl; and

R¹⁹ is OH, CN, C₁₋₄ alkyl, C₁₋₄ alkoxy, arylalkyl, heterocycloalkyl, aryl, heteroaryl, NR^{c}C(O)R^{d}, NR^{c}C(O)OR^{a}, C(O)R^{b}, C(O)NR^{c}R^{d} or C(O)OR^{a}, wherein each of said aryl or heteroaryl is optionally substituted by 1, 2 or 3 halo, CN, C₁₋₄ alkyl, C₁₋₄ alkoxy or C₁₋₄ haloalkyl.

In some embodiments, the compounds of the invention have Formula IV, and U¹ is NR¹⁷.

In some embodiments, the compounds of the invention have Formula IV, and U¹ is CR¹⁸R¹⁹.

In some embodiments, each —W—X—Y-Z is, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of said C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d};

In some embodiments, each —W—X—Y-Z is, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ haloalkoxy, C₁₋₆ alkyl, C₂₋₆ alkenyl, C₂₋₆ alkynyl, aryl, cycloalkyl, heteroaryl or heterocycloalkyl, wherein each of said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d}.

In some embodiments, each —W—X—Y-Z is, independently, OH, C₁₋₄ alkoxy, C₁₋₄ haloalkoxy, CN, C₁₋₆ alkyl, C₂₋₆ alkenyl, C₂₋₆ alkynyl, cycloalkyl, heterocycloalkyl, aryl, heteroaryl, NR^{c}C(O)R^{d}, NR^{c}C(O)OR^{a}, C(O)R^{b}, C(O)NR^{c}R^{d} or C(O)OR^{a}, wherein each of said aryl or heteroaryl is optionally substituted by 1, 2 or 3 halo, OH, CN, C₁₋₄ alkyl, C₁₋₄ alkoxy or C₁₋₄ haloalkyl.

In some embodiments, each —W—X—Y-Z is, independently, OH, CN, C₁₋₄ alkyl, C₁₋₄ alkoxy, arylalkyl, heterocycloalkyl, aryl, heteroaryl, NR^{c}C(O)R^{d}, NR^{c}C(O)OR^{a}, C(O)R^{b}, C(O)NR^{c}R^{d} or C(O)OR^{a}, wherein each of said aryl or heteroaryl is optionally substituted by 1, 2 or 3 halo, CN, C₁₋₄ alkyl, C₁₋₄ alkoxy or C₁₋₄ haloalkyl.

In some embodiments, each —W′—X′—Y′-Z′ is, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of said C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d}.

In some embodiments, each —W′—X′—Y′-Z′ is, independently, halo, CN, NO₂, OR^{a}, SR^{a}, C₁₋₄ haloalkoxy, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of said C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d}.

In some embodiments, each —W′—X′—Y′-Z′ is, independently, halo, CN, NO₂, OR^{a}, SR^{a}, C₁₋₄ haloalkoxy, C₁₋₆ alkyl, C₂₋₆ alkenyl, C₂₋₆ alkynyl, aryl, cycloalkyl, heteroaryl or heterocycloalkyl, wherein each of said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, CN, NO₂, OR^{a} or SR^{a}.

In some embodiments, each —W′—X′—Y′-Z′ is, independently, OH.

In some embodiments, each —W″—X″—Y″-Z″ is, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl, wherein each of said C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d}.

In some embodiments, each —W″—X″—Y″-Z″ is, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ haloalkoxy, C₁₋₆ alkyl, C₂₋₆ alkenyl, C₂₋₆ alkynyl, arylalkyl, cycloalkylalkyl, heteroarylalkyl, heterocycloalkylalkyl, aryl, cycloalkyl, heteroaryl or heterocycloalkyl.

In some embodiments, each —W″—X″—Y-Z″ is, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ haloalkoxy, C₁₋₆ alkyl, C₂₋₆ alkenyl, C₂₋₆ alkynyl, aryl, cycloalkyl, heteroaryl or heterocycloalkyl.

In some embodiments, each —W″—X″—Y″-Z″ is, independently, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}C(O)R^{d}, NR^{c}C(O)OR^{a}, C₁₋₄ haloalkoxy, C₁₋₆ alkyl, C₂₋₆ alkenyl, C₂₋₆ alkynyl, aryl, cycloalkyl, heteroaryl or heterocycloalkyl.

In some embodiments, each —W″—X″—Y″-Z″ is, independently, aryl, C(O)R^{b} or C(O)OR^{a}.

In some embodiments, Z, Z′ and Z″ are each, independently, H, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl or heterocycloalkyl, wherein each of said C₁₋₆ alkyl, C₂₋₆ alkenyl, C₂₋₆ alkynyl, aryl, cycloalkyl, heteroaryl or heterocycloalkyl is optionally substituted by 1, 2 or 3 halo, C₁₋₆ alkyl, C₂₋₆ alkenyl, C₂₋₆ alkynyl, C₁₋₄ haloalkyl, aryl, cycloalkyl, heteroaryl, heterocycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, or S(O)₂NR^{c}R^{d}.

In some embodiments, q is 1.

In some embodiments, q is 2.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R. In another example, when an optionally multiple substituent is designated in the form: 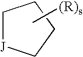
then it is understood that substituent R can occur s number of times on the ring, and R can be a different moiety at each occurrence. Further, in the above example, should the variable J be defined to include hydrogens, such as when J is said to be CH₂, NH, etc., any floating substituent such as R in the above example, can replace a hydrogen of the J variable as well as a hydrogen in any other non-variable component of the ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms. The term “alkylenyl” refers to a divalent alkyl linking group.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic cyclic hydrocarbons including cyclized alkyl, alkenyl, and alkynyl groups. Cycloalkyl groups can include mono- or polycyclic (e.g., having 2, 3 or 4 fused rings) groups. Ring-forming carbon atoms of a cycloalkyl group can be optionally substituted by oxo or sulfido. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like.

As used herein, “heterocyclyl”, “heterocyclic” or “heterocycle” refers to a saturated or unsaturated cyclic hydrocarbon wherein one or more of the ring-forming carbon atoms of the cyclic hydrocarbon is replaced by a heteroatom such as O, S, or N. Heterocyclyl groups can be aromatic (e.g., “heteroaryl”) or non-aromatic (e.g., “heterocycloalkyl”). Heterocyclyl groups can also correspond to hydrogenated and partially hydrogenated heteroaryl groups. Heterocyclyl groups can include mono- or polycyclic (e.g., having 2, 3 or 4 fused rings) ring systems. Heterocyclyl groups can be characterized as having 3-14 or 3-7 ring-forming atoms. In some embodiments, heterocyclyl groups can contain, in addition to at least one heteroatom, from about 1 to about 13, about 2 to about 10, or about 2 to about 7 carbon atoms and can be attached through a carbon atom or heteroatom. In further embodiments, the heteroatom can be oxidized (e.g., have an oxo or sulfido substituent) or a nitrogen atom can be quaternized. Examples of heterocyclyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like, as well as any of the groups listed below for “heteroaryl” and “heterocycloalkyl.” Further example heterocycles include pyrimidinyl, phenanthridinyl, phenanthrolinyl, phenazinyl, phenothiazinyl, phenoxathiinyl, phenoxazinyl, phthalazinyl, piperazinyl, piperidinyl, 3,6-dihydropyridyl, 1,2,3,6-tetrahydropyridyl, 1,2,5,6-tetrahydropyridyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, tetrahydrofuranyl, tetrahydroisoquinolinyl, tetrahydroquinolinyl, tetrazolyl, 6H-1,2,5-thia-diazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl, octahydro-isoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, quinazolinyl, quinolinyl, 4H-quinolizinyl, quinoxalinyl, quinuclidinyl, acridinyl, azocinyl, benzimidazolyl, benzofuranyl, benzothiofuranyl, benzo-thiophenyl, benzoxazolyl, benzthiazolyl, benztriazolyl, benztetrazolyl, benzisoxazolyl, benzisothiazolyl, benzimidazolinyl, methylenedioxyphenyl, morpholinyl, naphthyridinyl, deca-hydroquinolinyl, 2H,6H-1,5,2-dithiazinyl, dihydrofuro[2,3-b]tetrahydrofuran, furanyl, furazanyl, carbazolyl, 4aH-carbazolyl, carbolinyl, chromanyl, chromenyl, cinnolinyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl and isoxazolyl. Further examples of heterocycles include azetidin-1-yl, 2,5-dihydro-1H-pyrrol-1-yl, piperindin-1-yl, piperazin-1-yl, pyrrolidin-1-yl, isoquinol-2-yl, pyridin-1-yl, 3,6-dihydropyridin-1-yl, 2,3-dihydroindol-1-yl, 1,3,4,9-tetrahydrocarbolin-2-yl, thieno[2,3-c]pyridin-6-yl, 3,4,10,10a-tetrahydro-1H-pyrazino[1,2-a]indol-2-yl, 1,2,4,4a,5,6-hexahydro-pyrazino[1,2-a]quinolin-3-yl, pyrazino[1,2-a]quinolin-3-yl, diazepan-1-yl, 1,4,5,6-tetrahydro-2H-benzo[f]isoquinolin-3-yl, 1,4,4a,5,6,10b-hexahydro-2H-benzo[f]isoquinolin-3-yl, 3,3a,8,8a-tetrahydro-1H-2-aza-cyclopenta[a]inden-2-yl, and 2,3,4,7-tetrahydro-1H-azepin-1-yl, azepan-1-yl.

As used herein, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to non-aromatic heterocycles including cyclized alkyl, alkenyl, and alkynyl groups where one or more of the ring-forming carbon atoms is replaced by a heteroatom such as an O, N, or S atom. Heterocycloalkyl groups include monocyclic and polycyclic (e.g., having 2, 3 or 4 fused rings) systems. Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains 3 to about 14, 4 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double or triple bonds. In some embodiments, the heterocycloalkyl group contains 0 to 2 double or triple bonds.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used here, “haloalkoxy” refers to an —O-haloalkyl group. An example haloalkoxy group is OCF₃.

As used herein, “pentahalosulfanyl” refers to moieties of formula —SX₅ where each X is independently selected from F, Cl, Br, or I. For methods of preparing compounds containing pentahalosulfanyl groups see, e.g., Org. Lett. 2002, 4, 3013.

As used herein, “aryloxy” refers to an —O-aryl group. An example aryloxy group is phenoxy.

As used herein, “arylalkyl” refers to alkyl substituted by aryl and “cycloalkylalkyl” refers to alkyl substituted by cycloalkyl. An example arylalkyl group is benzyl.

As used herein, “heteroarylalkyl” refers to alkyl substituted by heteroaryl and “heterocycloalkylalkyl” refers to alkyl substituted by heterocycloalkyl.

As used herein, “amino” refers to NH₂.

As used herein, “alkylamino” refers to an amino group substituted by an alkyl group.

As used herein, “dialkylamino” refers to an amino group substituted by two alkyl groups.

As used herein, “N-substituted piperidin-3-yl” refers to a moiety having the formula: 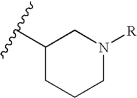
wherein R is any moiety other than H. In general, the terms “substitute” or “substitution” refer to replacing a hydrogen with a non-hydrogen moiety.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include all potential tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention further include solid forms which are crystalline, amorphous, hydrated, solvated, anyhydrous, or non-solvated.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention can be in isolated form. An isolated compound is one that has been at least partially or substantially separated from the environment in which is was formed or discovered.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The present invention also includes prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Synthesis

The novel compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the invention can be prepared, for example, using the reaction pathways and techniques as described below.

A series of N-(piperidin-3-yl)carboxamides of formula 4 and 4′ can be prepared by the method outlined in Scheme 1. 1-(tert-Butoxycarbonyl)-3-amino-piperidine 1 can be coupled to an acid chloride R³COCl in the presence of a base such as Hunig's base or potassium carbonate to provide the desired product 2. Alternatively, the amide coupling of compound 1 with an acid R³COOH can be conducted by utilizing conventional coupling agents such as BOP, DIC, EDCI, DCC, PyBOP, or triazine coupling agents (Kunishima, M. et al. Tetrahedron 1999, 55, 13159). The Boc protecting group of compound 2 can be removed by treatment with an acid such as TFA or HCl in 1,4-dioxane to afford the amino salt 3, which can be directly coupled with the appropriate chloride CyLCl to give the final compounds of formula 4, wherein L can be SO₂ or CO. Alternatively, urea compounds 4′ can be prepared via the activated p-nitro-carbamate or carbonyl-3-methyl-1H-imidazol-3-ium species (intermediates I-3 where A is 4-nitrophenoxy or 3-methylimidazol-1-yl). Alternatively, the piperidine 3 can be reacted with an appropriate carbamoyl chloride R^{A}R^{B}NC(O)Cl or isocyanate R^{A}R^{B}N═C═O to afford urea compounds 4′.

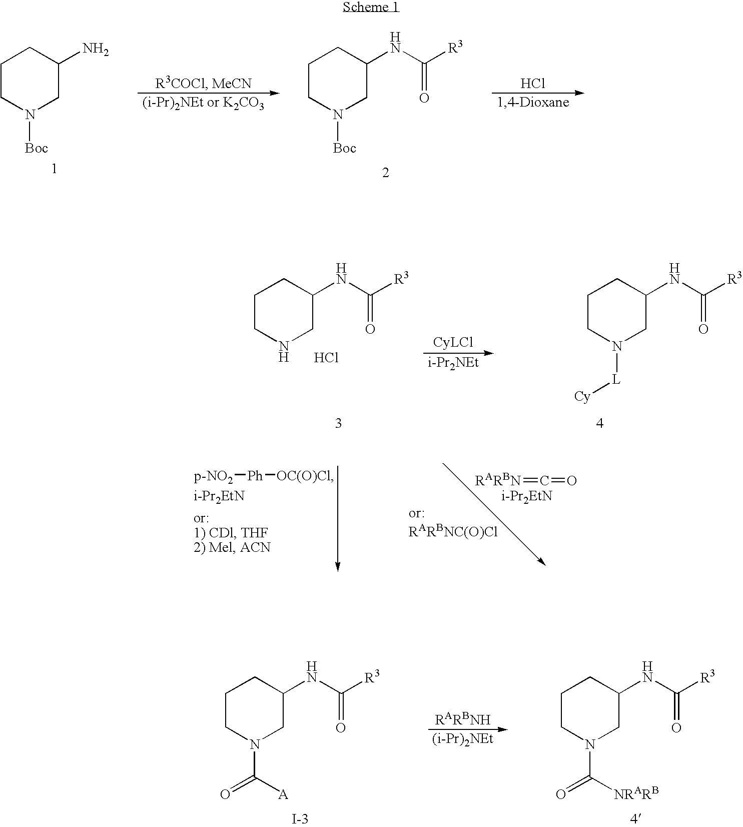

Alternatively, the same series of N-(piperidin-3-yl)carboxamides of formula 4 and 4′ can be prepared by reversing the coupling sequences as depicted in Scheme 2 (where A is 4-nitrophenoxy or 3-methylimidazol-1-yl).

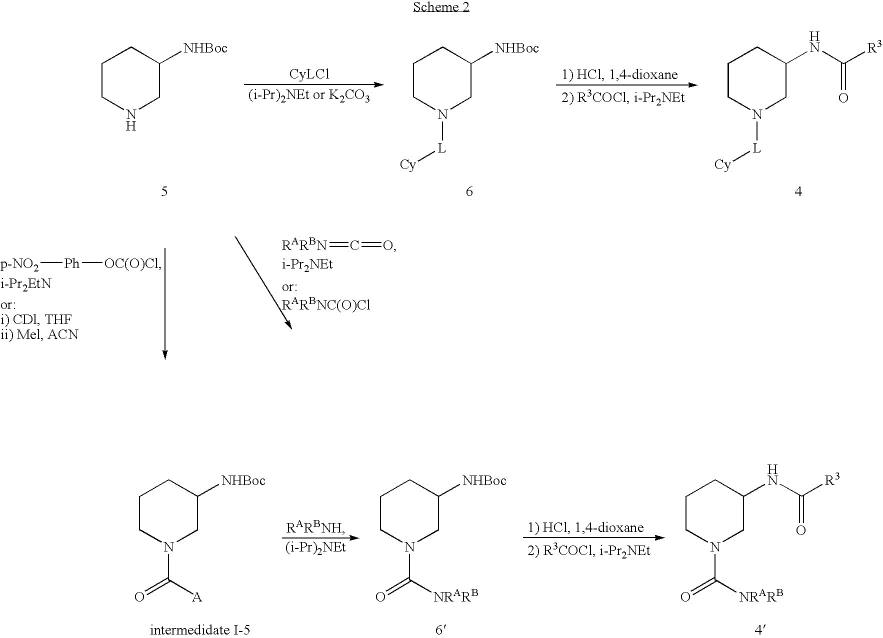

A series of 5-substituted 3-aminopiperidines of formula 13 can be prepared according to the method outlined in Scheme 3. L-Glutamic acid dimethyl ester 7 was protected by reaction with di-tert-butyl dicarbonate to afford the N-Boc protected compound 8. The dianion enolate of compound 8 can be formed in the presence of a suitable base such as sodium hydride, LDA, or LiHMDS and in a suitable solvent such as THF and then coupled with an electrophile RX such as an alkylhalide or alkyltriflate to provide 4-alkyl dimethyl ester 9. Reduction of the ester groups with a suitable reducing reagent such as NaBH₄/CaCl₂ affords the di-alcohol compound 10. Subsequent conversion of the hydroxyl groups of compound 10 to leaving groups such as tosyl or mesyl groups followed by reaction with an appropriate primary amine such as BnNH₂ affords the 5-substituted 3-aminopiperidine 12, which can be deprotected and derivatized by the methods previously described.

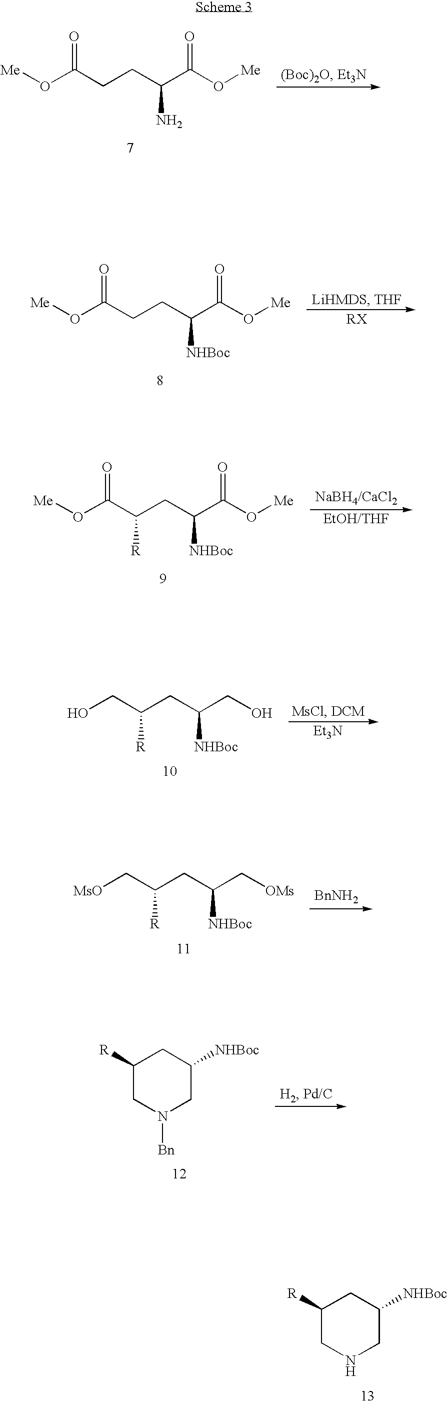

As shown in Scheme 4, a series of spiro-3-aminopiperidines of formula 19 can be prepared utilizing a similar synthetic strategy to that described above by reacting the dianion enolate of compound 14 with a reagent R-14, i.e., an alkyl chain that has two leaving groups such as halides or alcohol derivatives (i.e., tosyl, mesyl, etc). For example, reagents R-14 can be 1,2-di-bromoethane or 1,3-di-bromopropane.

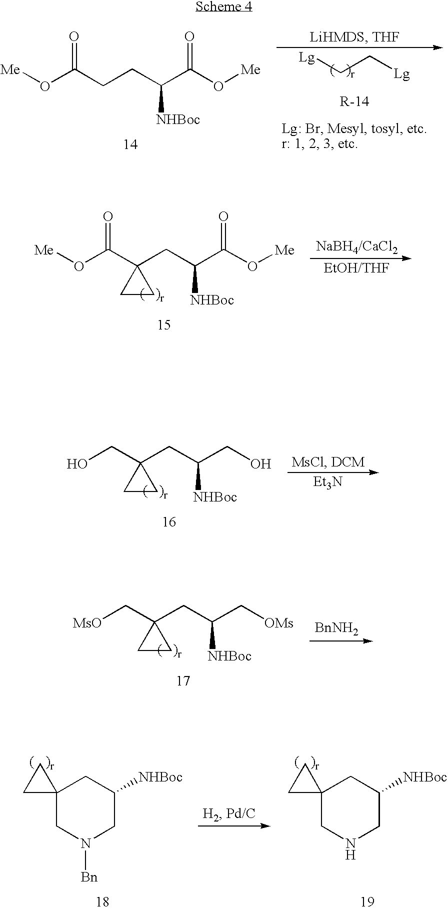

A series of 3-substituted-3-aminopiperidines of formula 25 can be prepared according to the method outlined in Scheme 5, wherein R¹ can be alkyl, aryl, arylalkyl, cycloalkyl or cycloalkylalkyl. Ketone 20 can be treated with TsNH₂ to give the imino compound 21, which can be subsequently reacted with an electrophile such as a Grignard reagent to afford a Ts-protected-amine compound 22. The Ts protecting group of compound 22 can then be removed by treatment with PhSH and replaced with a more labile Boc-protecting group by treatment with (Boc)₂O in the presence a suitable base such as triethylamine to afford compound 24. The Bn group of compound 24 is removed by palladium mediated hydrogenation to afford the desired 3-substituted-3-aminopiperidine intermediate 25, which can then be derivatized accordingly by methods previously described herein.

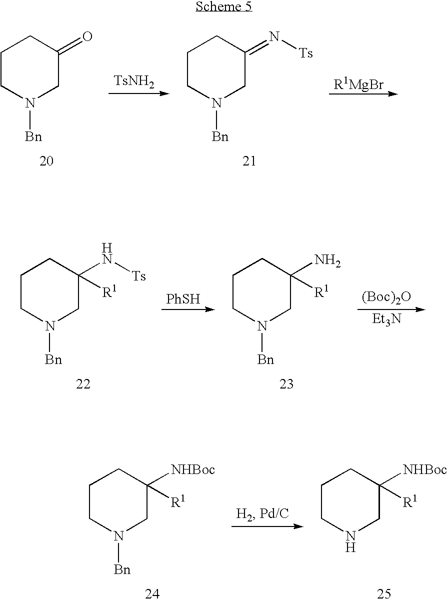

Tertiary amides of formula 28 can be prepared as shown in Scheme 6, wherein Q is SO₂Cy, CO₂Cy, or C(O)NR^{A}R^{B}. Reductive amination of 3-aminopiperidine 26 with a suitable aldehyde R′CHO, wherein R′ is alkyl, aryl, heteroaryl, cycloalkyl, heterocycloalkyl, arylalkyl and the like, affords a secondary amine 27. Subsequent amide coupling of amine 27 with a carboxylic acid R³COOH (via activation by a coupling reagent such as BOP) provides the tertiary amide 28.

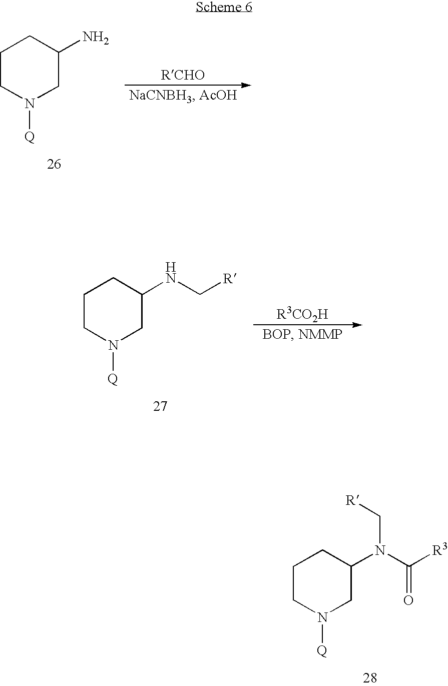

Alternatively, a series of N-(piperidin-3-yl)carboxamides of formula 30 can be prepared by the method outlined in Scheme 7, wherein R² can be alkyl or cycloalkyl. An alkyl or cycloalkyl group R² can be directly introduced to the N-atom of the secondary amide 29 to form the desired tertiary amide 30 under the conditions of phase transfer catalysis by using a suitable catalyst such as tributylammonium bromide.

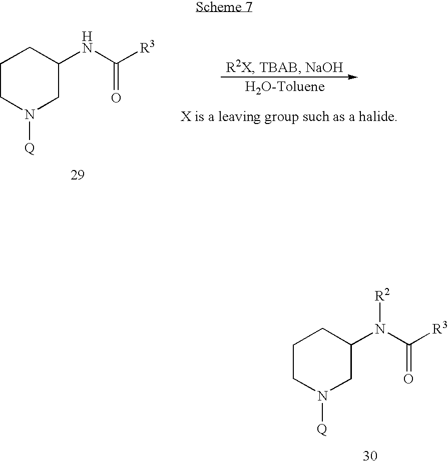

A series of carboxamides of formula 34 (wherein A is S, O, CH₂ or NR′; R′ is alkyl, cycloalkyl, arylalkyl, etc.; s is 1, 2 or 3; and t is 1 or 2) can be prepared according to the method outlined in Scheme 8, wherein R can be alkyl, aryl, arylalkyl, or the like and X is a leaving group such as halo. Formation of the ester enolate of compound 31 can be facilitated by treatment with a base such as sodium hydride, LiHMDS, or LDA and in a suitable solvent such as DMF or THF. Subsequent reaction of the enolate with an electrophile, such as an alkyl halide affords an R-substituted ester 32, which upon basic hydrolysis yields carboxylic acid 33. Activation of the carboxylic acid 33 by treatment with a reagent such as thionyl chloride, DIC, or BOP reagent followed by condensation with the 3-aminopiperidine 26 affords the desired amide 34.

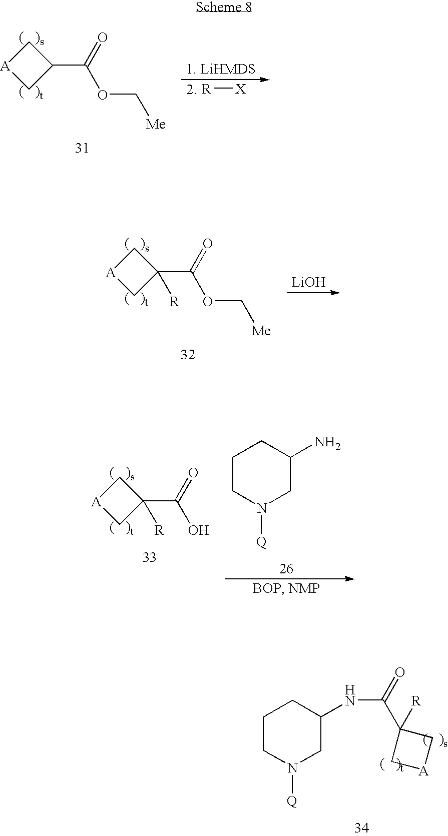

Due to the plethora of available carboxylic acids, an abundance of carboxamides can be prepared with a wide range of structural diversity. The following schemes illustrate typical synthetic methodologies that can be used to prepare a variety of carboxylic acids that can be subsequently coupled to the 3-aminopiperidine by using procedures analogous to those disclosed herein.

A series of carboxylic acids of formula 38 can be prepared according to the method outlined in Scheme 9, wherein J can be S, O, or NR; R can H, alkyl, or the like; R′ and R″ can be independently alkyl or arylalkyl; and Cy² can be aryl, heteroaryl, cycloalkyl or heterocycloalkyl. Reaction of an appropriate thiol, alcohol, or amine 35 with methyl bromoacetate in the presence of a suitable base such as potassium or sodium carbonate, triethylamine or sodium hydride in a suitable solvent such as tetrahydrofuran, acetonitrile or dichloromethane provides a thioether, ether, or amine compound 36. Treatment of compound 36 with R′X and R″X (R′X and R″X can be the same or different, such as alkyl halides or activated alcohol, e.g. tosylate, mesylate, etc.) in the presence of a suitable base such as sodium hydride or LDA and in a suitable solvent such as DMF or THF provides ester compound 37, which upon basic hydrolysis yields the desired carboxylic acid 38.

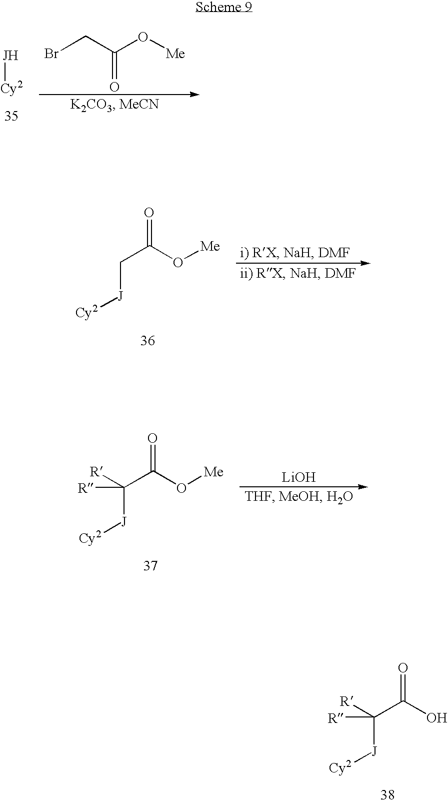

R′ and R″ described in Schemes 9 can be alkyl chains or R′ and R″ together with the carbon atom to which they are attached can form a cycloalkyl or heterocycloalkyl, group (ring T) such that the alkylation of the enolate of ester 36 affords compound 37′ as depicted in Scheme 10.

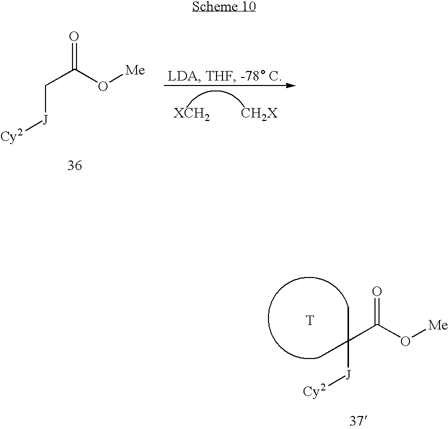

α,β-Unsaturated, aromatic, and heteroaromatic carboxylic acids derivitization can be accomplished by conventional methods such as conjugate addition, electrophilic aromatic substitution, stereoselective reduction, and transition metal catalyzed coupling reactions, particularly palladium-catalyzed cross coupling reactions (Nicolaou, K. C.; Bulger, P. G.; Sarlah, D. Angew. Chem. Int. Ed. 2005, 44, 4442).

As shown in scheme 11, ortho-amino-pyridine carboxylic acids of the general formula 39 and 39′ can be prepared by heating the corresponding ortho-halopyridine compound 38 in the presence of an appropriate amine R′R″NH (wherein R′ and R″ can be independently alkyl, cycloalkyl, heteocycloalkyl, aromatic, heteroaromatic, etc.; X can be halo or triflate, etc.; Y is cyano, alkyl, haloalkyl, etc.) or an NH-containing heterocyclic compound R-38 such as piperidine or morpholine [von Geldern, Thomas W. et al. Biorg. & Med. Chem. Lett. 2005, 15, 195].

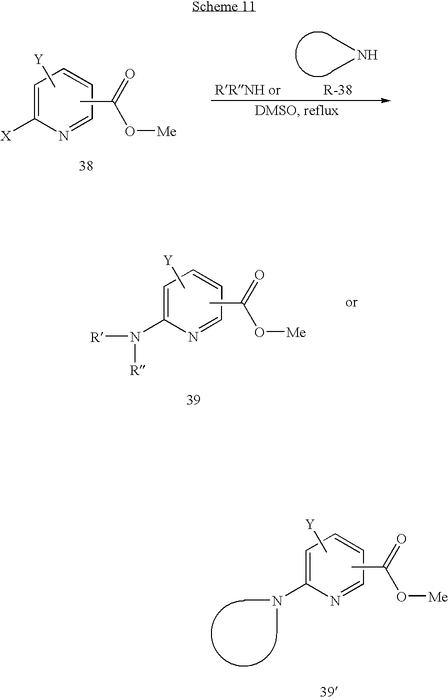

As shown in Scheme 12, alternatively, conventional aromatic/amine metal mediated coupling reactions of compounds 40 and 41 can be implemented when compound 40 is other than an ortho-halo-pyridine derivative (i.e., W is N and X is a halo or triflate group at the ortho position to W), wherein X is, e.g., Cl, Br, I, OTf, etc.; W is N or CH; Q is O, NH, N(alkyl), CH₂, CH(alkyl), C(alkyl)₂, etc.; and R^{II} and R^{III} are independently H, alkyl, cycloalkyl, aromatic, heteroaromatic, etc.; or R^{II} and R^{III} together with the C(=Q)NH to which they are attached form a heterocycle. For example, copper (I) mediated coupling reactions can be used when the NH group of compound 41 is α to an sp² carbon such as in the case of a pyrazole, oxazolidin-2-one, 2-oxo-pyrrolidine, imidazole, indazole, 1H-benzimidazole, pyrid-2-one, t-butyl carbamate, etc. according to Scheme 12. (Woolven, James M. et al. J. Med. Chem. 2003, 46, 4428).

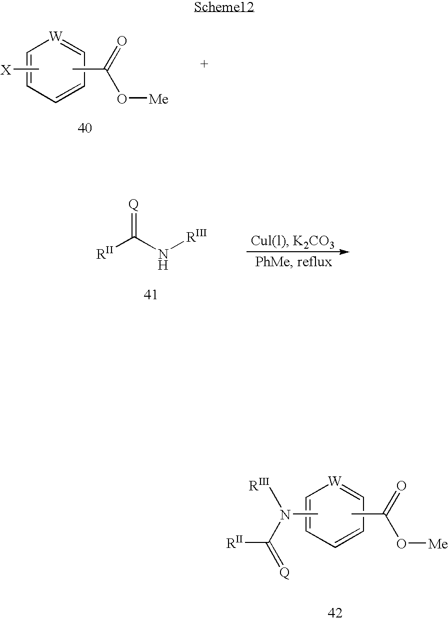

In addition to the abundance of carboxylic acids that are readily available, there is also a plethora of readily available amines that can be used for the synthesis of the compounds of the invention as shown in Scheme 13 (where A is 4-nitrophenoxy or 3-methylimidazol-1-yl). For example, a variety of amines R^{3a}R^{3b}NH can be used for making the intermediate 2′, and a variety of amines R^{A}R^{B}NH can be used for making compounds 4′ and 6′.

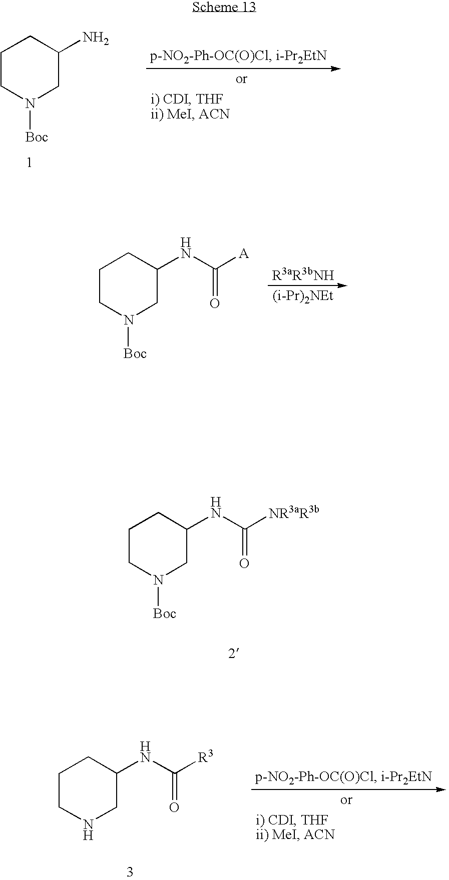

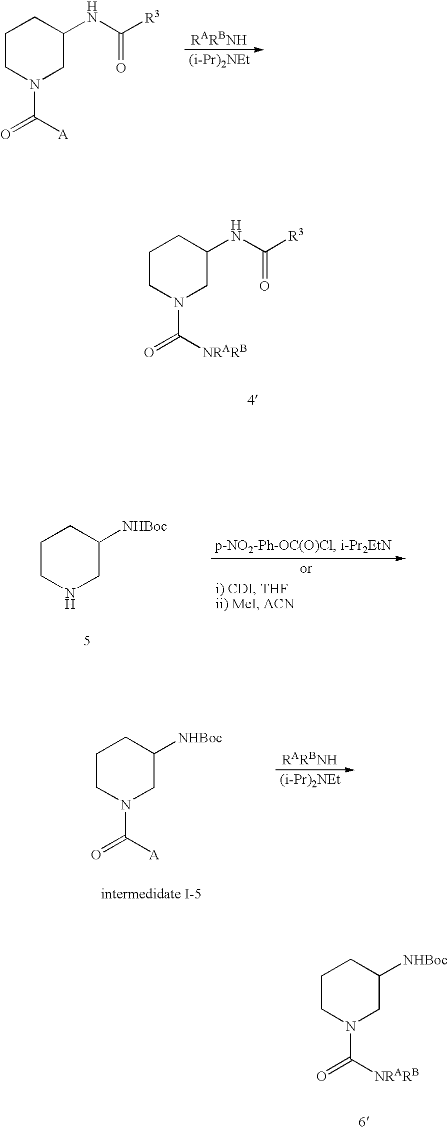

Spiro-pyrrolidines 45 can be prepared according to Scheme 14. Halogen/metal exchange between aryl iodide 43 and isopropylmagnesium bromide followed by reaction with N-Boc-3-oxo-pyrrolidine provides spiro-lactone 44 which upon acidic cleavage of the Boc group yields the desired pyrrolidine 45.

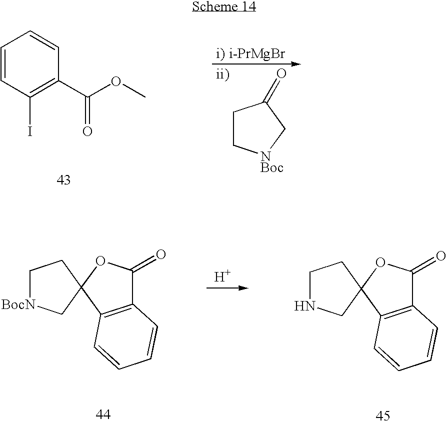

Spiro-pyrrolidines 48 can be prepared according to Scheme 15. ortho-Lithiation of carboxylic acid 46 followed by reaction of the resulting organolithium species with N-Boc-3-oxo-pyrrolidine yields spiro-lactone 47, which upon acidic cleavage of the Boc group provides the desired pyrrolidine 48.

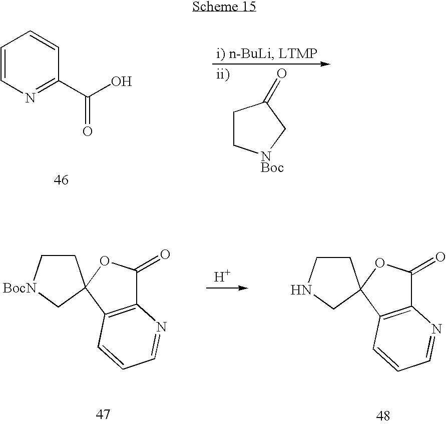

Spiro-pyrrolidine 53 can be prepared according to the rearrangement method outlined in Scheme 16.

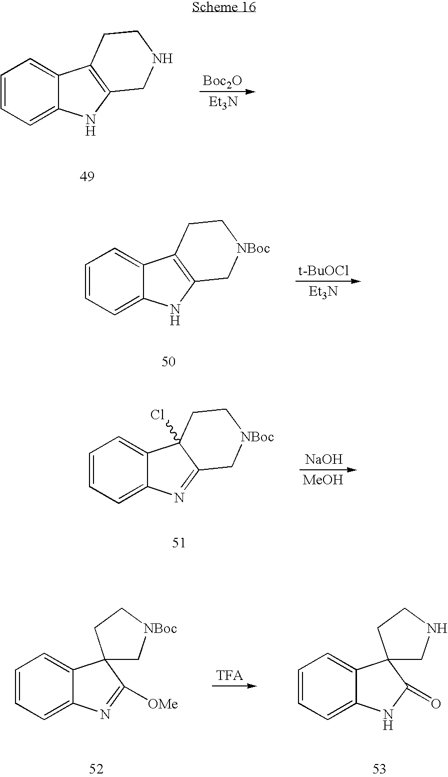

A series of 3-substituted pyrrolidines 56 and 58 and pyrrolid-3-enes 57 can be prepared by the method outlined in Scheme 17 (R^{x} can be, for example, alkyl or cycloalkyl). Compound 54 can be treated with an organolithium or Grignard reagent to provide alcohol 55. The Boc protecting group of 55 can be removed by treatment with an acid such as TFA to afford the 3-substituted pyrrolidine 56. Alternatively, 55 can be treated with HCl to provide the pyrrolid-3-ene 57, which can be subsequently reduced by Pd-catalyzed hydrogenation to afford 3-substituted pyrrolidine 58.

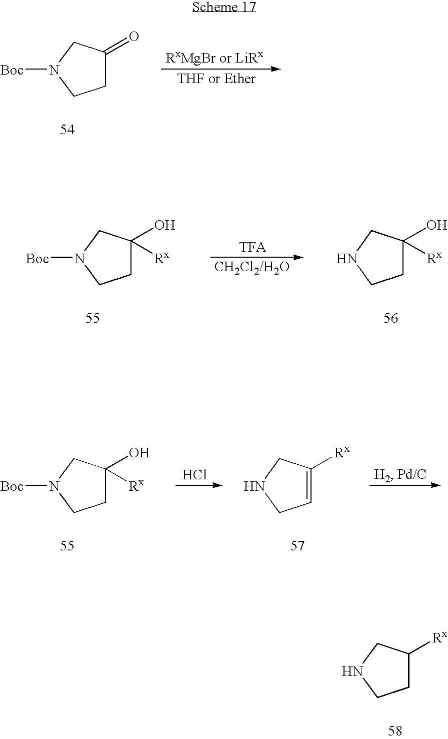

A series of 3-substituted pyrrolidines 60 can be prepared by the method outlined in Scheme 18 (Ar can be, for example, aryl or heteroaryl). Palladium catalyzed Heck coupling reaction of alkene 59 with arylbromides or heteroarylbromides followed by hydrogenation to remove the Cbz group provides the desired 3-substituted pyrrolindine 60 (Ho, C. et al Tetrahedron Lett. 2004, 45, 4113).

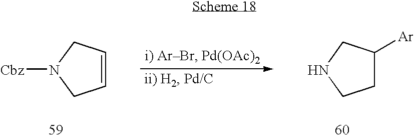

A series of 3-hydroxyl-4-substituted pyrrolidines 62 can be prepared by the method outlined in Scheme 19 (wherein Ar can be, for example, aryl or heteroaryl; X can be halo). Alkene 59 can be reacted with MCPBA to provide the corresponding epoxide, which is subsequently reacted with an organolithium reagent in the presence of a Lewis acid, such as AM(Me)₃, and followed by hydrogenation to remove the Cbz group, to provide the desired 3-hydroxyl-4-substituted pyrrolindine 62.

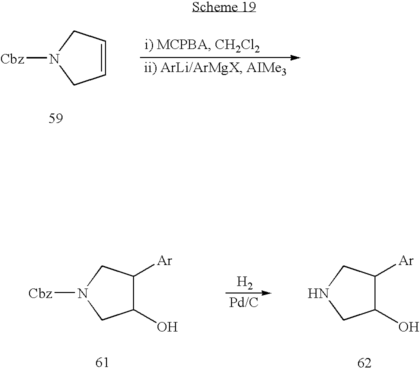

A series of di-substituted nitrogen-containing heterocycles of formula 66 can be prepared by the method outlined in Scheme 20 (wherein Ar is, for example, aryl or heteroaryl; m and n are independently, 0, 1, 2 3 or 4, but both can not be 0 simultaneously). Ketone 63 can be treated with a Wittig reagent to provide vinyl compound 64, which can be reacted with Ar₂CuLi to provide the 1,4-addition product 65. The Cbz protecting group of 65 can be removed by hydrogenation to provide the desired di-substituted nitrogen-containing heterocycle 66. Alternatively, the alkene 64 can be reduced under asymmetric homogeneous catalyzed hydrogenation to afford compound 65′ or compound 65″, which can be subjected to further hydrogenation to afford compound 66′ or compound 66″. In some instances, compound 64 can be reduced under asymmetric homogeneous catalyzed hydrogenation to afford compound 66′ or compound 66″ directly.

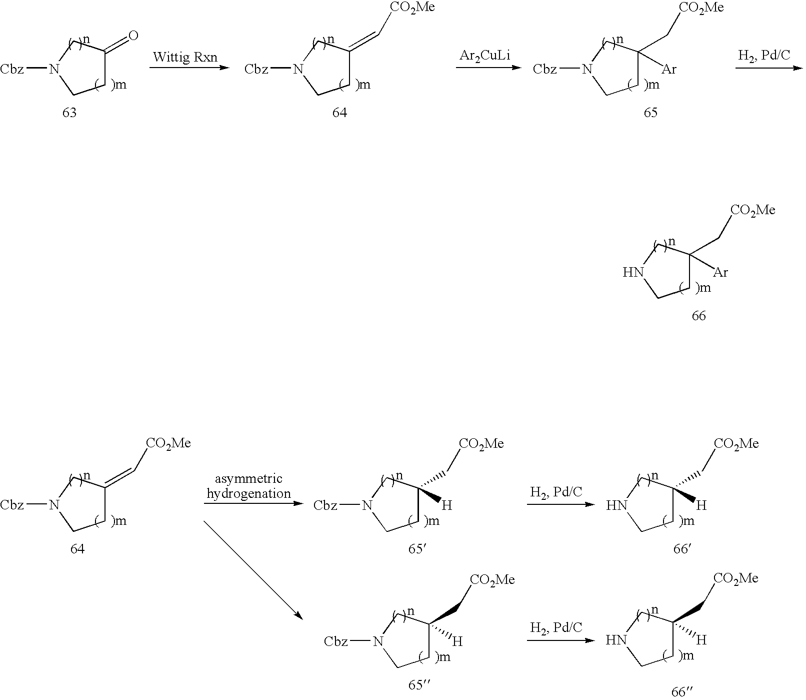

A series of aromatic piperazine intermediates 71 can be prepared according to Scheme 21, wherein Lv is a leaving group such as Cl, Br, I or OTf; R can be CN, alkyl, haloalkyl or the like; and G is N or CH. Boc-piperazine 67 can be reacted with a variety of boronic acids 68 under the catalysis of copper (II) acetate (Combs, A. P.; Tadesse, S.; Rafalski, M.; Haque, T. S.; Lam, P. Y. S. J. Comb. Chem. 2002, 4, 179) or with a variety of aryl or heteroaryl halides 69 using Buchwald/Hartwig conditions (Louie, J; Hartwig, J. F. Tetrahedron Lett. 1995, 36, 3609 & Bolm, C. et al. J. Org. Chem. 2005, 70, 2346.). Removal of the Boc group of compound 70 with TFA affords the desired the secondary amine 71. Alternatively, the aromatic piperazine compounds 70 or 71 can also be prepared through classical ring closure of appropriately substituted anilines and bis-(2-chloroethyl)amine hydrochloride in the presence of base (E. Mishani, et. al. Tetrahedron Lett. 1996, 37, 319), or through direct nucleophilic aromatic substitution of the piperazine (S. M. Dankwardt, et al., Tetrahedron Lett. 1995, 36, 4923).

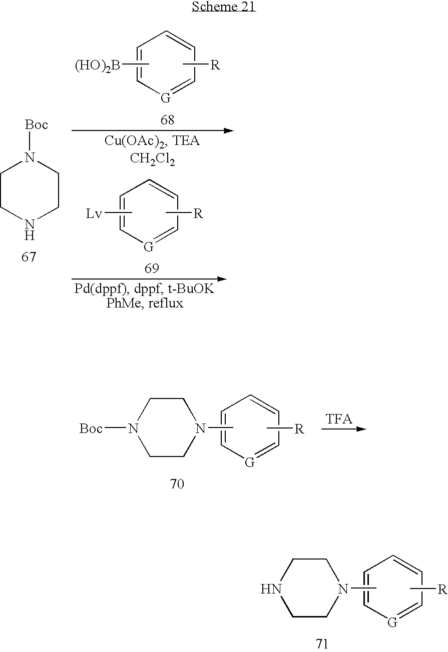

A series of aryl- or heteroaryl-tetrahydropyridines 74 can be prepared by first converting the tert-butoxycarbonyl-piperid-4-one 72 to the corresponding enol triflate 75 using LDA and N-phenyltrifluoromethanesulfonamide according to Scheme 22. The enol triflate 75 can then be used directly in a Suzuki-type coupling reaction with a variety of aromatic boronic acids 68 to produce the aryl- or heteroaryl-tetrahydropyridines 76, wherein G is either N or CH (M. G. Bursavich, D. H. Rich, Org. Lett. 2001, 3, 2625). Alternatively, the enol triflate 75 can be converted to the corresponding enol boronic ester 77 (or a corresponding enol boronic acid) via palladium mediated coupling and then subsequently coupled with an aryl-heteroaryl-halide 69 through a Suzuki-type reaction. Finally, the Boc protecting group of compound 76 can be removed by treatment with an acid such as TFA to afford the desired 4-aryl tetrahydropyridine 74.

The 4-aromatic tetrahydropyridines 74 can also be prepared through alternative methods known by those skilled in the art of organic synthesis, such as direct nucleophilic addition of an anion of aryl or heteroaryl 69 (through metal/halide exchange) to a piperidone 72 afford an alcohol compound 73, which is subsequently subjected to dehydration and removing of the Boc group to afford compound 74.

In addition, hydrogenation of the 4-aryl tetrahydropyridine 74 can provide the corresponding 4-aryl- or 4-heteroaryl-piperidine compound.

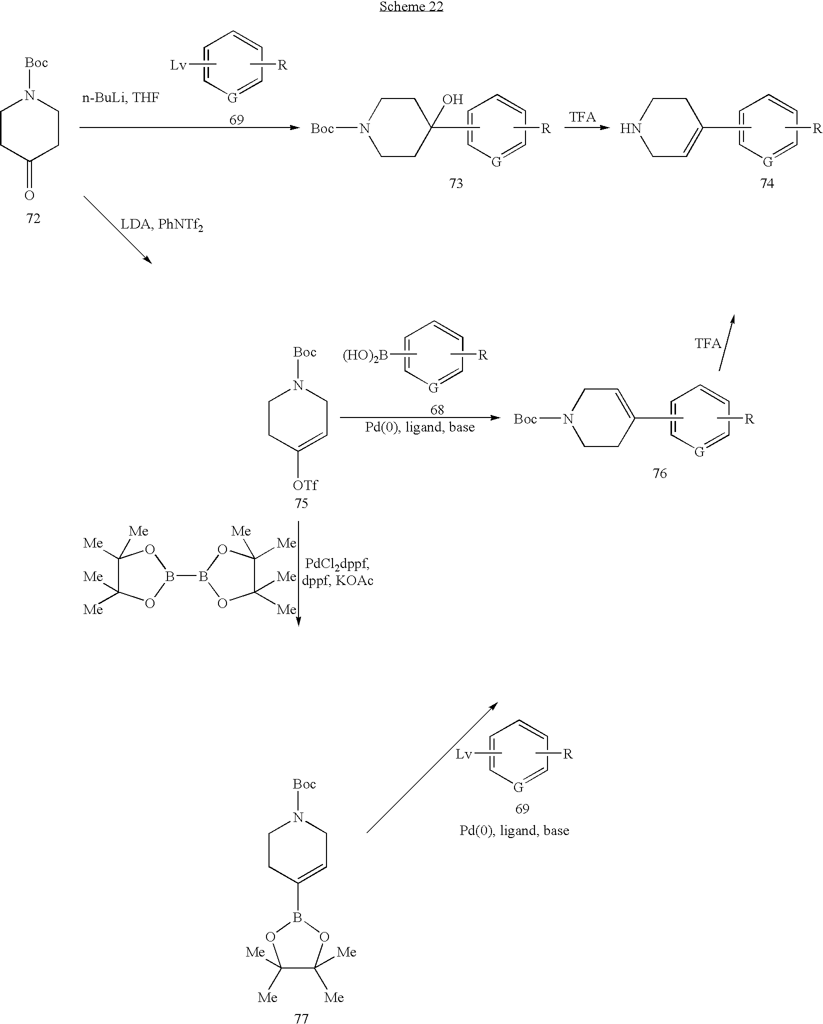

A series of aromatic piperidine derivatives 79 can be prepared according to Scheme 23, wherein Lv is a leaving group like halo; G is CH or N; R can be CN, alkyl, haloalkyl or the like. Suzuki coupling of 4-bromopyridine with an aromatic boronic acid 68 followed by hydrogenation affords the desired piperidine derivative 79. 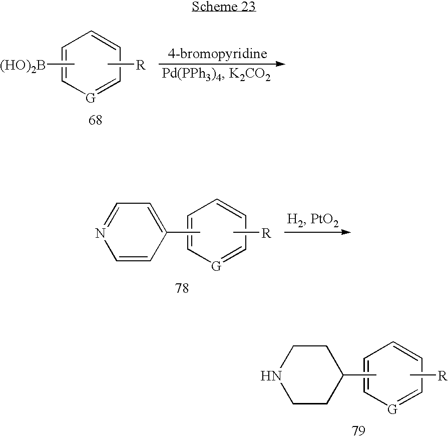
Methods

Compounds of the invention can modulate activity of 11βHSD1. The term “modulate” is meant to refer to an ability to increase or decrease activity of an enzyme. Accordingly, compounds of the invention can be used in methods of modulating 11βHSD1 by contacting the enzyme with any one or more of the compounds or compositions described herein. In some embodiments, compounds of the present invention can act as inhibitors of 11βHSD1. In further embodiments, the compounds of the invention can be used to modulate activity of 11βHSD1 in an individual in need of modulation of the enzyme by administering a modulating amount of a compound of the invention.

The present invention further provides methods of inhibiting the conversion of cortisone to cortisol in a cell, or inhibiting the production of cortisol in a cell, where conversion to or production of cortisol is mediated, at least in part, by 11βHSD1 activity. Methods of measuring conversion rates of cortisone to cortisol and vice versa, as well as methods for measuring levels of cortisone and cortisol in cells, are routine in the art.

The present invention further provides methods of increasing insulin sensitivity of a cell by contacting the cell with a compound of the invention. Methods of measuring insulin sensitivity are routine in the art.

The present invention further provides methods of treating disease associated with activity or expression, including abnormal activity and overexpression, of 11βHSD1 in an individual (e.g., patient) by administering to the individual in need of such treatment a therapeutically effective amount or dose of a compound of the present invention or a pharmaceutical composition thereof. Example diseases can include any disease, disorder or condition that is directly or indirectly linked to expression or activity of the enzyme or receptor. An 11βHSD1-associated disease can also include any disease, disorder or condition that can be prevented, ameliorated, or cured by modulating enzyme activity.

Examples of 11βHSD1-associated diseases include obesity, diabetes, glucose intolerance, insulin resistance, hyperglycemia, hypertension, hyperlipidemia, cognitive impairment, dementia, depression (e.g., psychotic depression), glaucoma, cardiovascular disorders, osteoporosis, and inflammation. Further examples of 11βHSD1-associated diseases include metabolic syndrome, coronary heart disease, type 2 diabetes, hypercortisolemia, androgen excess (hirsutism, menstrual irregularity, hyperandrogenism) and polycystic ovary syndrome (PCOS).

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal. In some embodiments, the cell is an adipocyte, a pancreatic cell, a hepatocyte, neuron, or cell comprising the eye.

As used herein, the term “contacting” refers to the bringing together of indicated moieties in an in vitro system or an in vivo system. For example, “contacting” the 11βHSD1 enzyme with a compound of the invention includes the administration of a compound of the present invention to an individual or patient, such as a human, having 11βHSD1, as well as, for example, introducing a compound of the invention into a sample containing a cellular or purified preparation containing the 11βHSD1 enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition or disorder; and

(3) ameliorating the disease; for example, ameliorating a disease, condition or disorder in an individual who is experiencing or displaying the pathology or symptomatology of the disease, condition or disorder.

Pharmaceutical Formulations and Dosage Forms

When employed as pharmaceuticals, the compounds of Formula I can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, antibodies, immune suppressants, anti-inflammatory agents and the like.

Labeled Compounds and Assay Methods

Another aspect of the present invention relates to labeled compounds of the invention (radio-labeled, fluorescent-labeled, etc.) that would be useful not only in radio-imaging but also in assays, both in vitro and in vivo, for localizing and quantitating the enzyme in tissue samples, including human, and for identifying ligands by inhibition binding of a labeled compound. Accordingly, the present invention includes enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I , ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I , ³⁵S and ⁸²Br.

Other labeled compound of the present invention contains a fluorescent label.

Synthetic methods for incorporating radio-isotopes into organic compounds are applicable to compounds of the invention and are well known in the art.

A labeled compound of the invention (radio-labeled, fluorescent-labeled, etc.) can be used in a screening assay to identify/evaluate compounds. For example, a newly synthesized or identified compound (i.e., test compound) which is labeled can be evaluated for its ability to bind a 11βHSD1 or MR by monitoring its concentration variation when contacting with the 11βHSD1 or MR, through tracking the labeling. For another example, a test compound (labeled) can be evaluated for its ability to reduce binding of another compound which is known to bind to 11βHSD1 or MR (i.e., standard compound). Accordingly, the ability of a test compound to compete with the standard compound for binding to the 11βHSD1 or MR directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

Kits

The present invention also includes pharmaceutical kits useful, for example, in the treatment or prevention of 11βHSD1- or MR-associated diseases or disorders, obesity, diabetes and other diseases referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. The compound of the Examples were found to inhibitors of 11βHSD1 and/or MR according to one or more of the assays provided herein.

### EXAMPLES

### Example 1

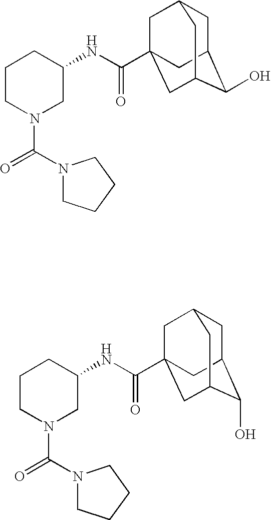

### 4-Hydroxy-N-[(3S)-1-(pyrrolidin-1-ylcarbonyl)piperidin-3-yl]adamantane-1-carboxamide

Step 1: tert-Butyl (3S)-3-{[(4-oxo-1-adamantyl)carbonyl]amino}piperidine-1-carboxylate

Oxalyl chloride (233 μL, 0.00275 mol) was added to 4-oxoadamantane-1-carboxylic acid (97.08 mg, 0.0004998 mol) in methylene chloride (10 mL) at rt followed by 2 drops of DMF. After stirring the mixture at rt for 2 h, the volatiles were evaporated under reduced pressure. The residue was azeotropically evaporated twice with toluene and the resulting residue was dissolved in DCM (10 mL). To the solution was added tert-butyl (3S)-3-aminopiperidine-1-carboxylate (100.1 mg, 0.0004998 mol) and N,N-diisopropylethylamine (0.18 mL, 0.0010 mol). After stirring at rt for 1 h, the reaction mixture was diluted with DCM (100 mL) and washed with water, 1N HCl, and brine. The organic phase was dried over Na₂SO₄, filtered, and concentrated in-vacuo to provide the desired product. LCMS: (M -t-Bu+H)⁺=321.2.

Step 2: tert-butyl (3S)-3-{[(4-hydroxy-1-adamantyl)carbonyl]amino}piperidine-1-carboxylate

1.0 M of L-selectride ® in tetrahydrofuran (0.50 mL) was added to a solution of tert-butyl (3S)-3-{[(4-oxo-1-adamantyl)carbonyl]amino}piperidine-1-carboxylate (75 mg, 0.00020 mol) in tetrahydrofuran (1.0 mL, 0.012 mol) at −78° C. The mixture was stirred at −78° C. for 30 min. and was then quenched with ice-water. The mixture was extracted with ethyl acetate (3×2 mL). The combined organic phases were washed with brine (2 mL), dried over Na₂SO₄, filtered, and concentrated under reduced pressure. The residue was purified by Combiflash, eluting with ethyl acetate/hexanes, to provide the desired product. LCMS: (M -t-Bu+H)⁺=323.2.

Step 3: 4-Hydroxy-N-[(3S)-piperidin-3-yl]adamantane-1-carboxamide hydrochloride

tert-Butyl (3S)-3-{[(4-hydroxy-1-adamantyl)carbonyl]amino}piperidine-1-carboxylate (75 mg, 0.00020 mol) was treated with 4.0 M of hydrogen chloride in 1,4-dioxane (0.30 mL) at rt for 30 min. The volatiles were evaporated and the residue was dried under reduced pressure to afford the desired product, which was used in the subsequent step without further purification. LCMS: (M+H)⁺=315.4.

Step 4: 4-Hydroxy-N-[(3S)-1-(pyrrolidin-1-ylcarbonyl)piperidin-3-yl]adamantane-1-carboxamide

A mixture of 4-hydroxy-N-[(3S)-piperidin-3-yl]adamantane-1-carboxamide (13.9 mg, 0.0000500 mol), 1-pyrrolidinecarbonyl chloride (10.0 mg, 0.0000750 mol) and N,N-diisopropylethylamine (19.4 mg, 0.000150 mol) in acetonitrile (0.75 mL, 0.014 mol) was stirred at rt for 1 h. The mixture was adjusted with TFA to pH=2.0 and was diluted with methanol (1.0 mL). The resulting solution was purified by prep.-HPLC to afford both of the desired equatorial and axial hydroxyl diastereoisomer products. LCMS: (M+H)⁺=376.2.

### Example 2

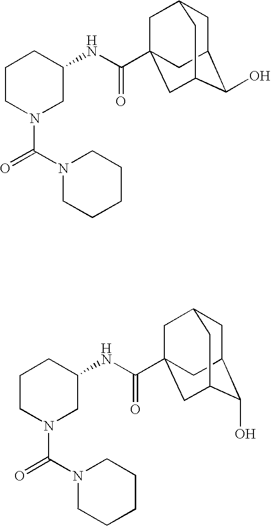

### 4-Hydroxy-N-[(3S)-1-(piperidin-1-ylcarbonyl)piperidin-3-yl]adamantane-1-carboxamide

This compound was prepared using a procedure that was analogous to that described for the synthesis of example 1, steps 1-4. LCMS: (M+H)⁺=390.3.

### Example 3

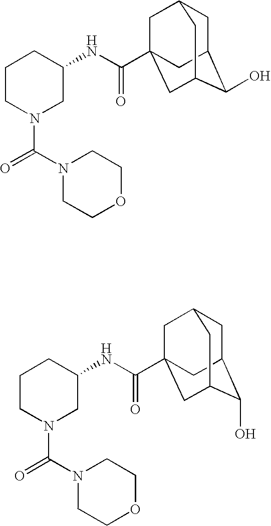

### 4-Hydroxy-N-[(3S)-1-(morpholin-4-ylcarbonyl)piperidin-3-yl]adamantane-1-carboxamide

This compound was prepared using a procedure that was analogous to that described for the synthesis of example 1, steps 1-4. LCMS: (M+H)⁺=392.3.

### Example 4

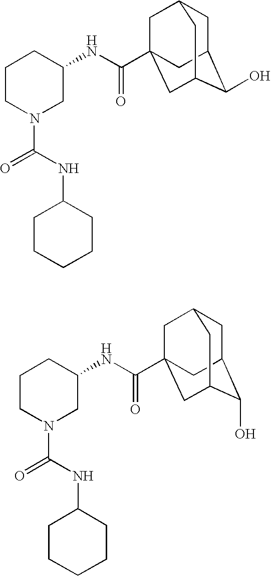

### (3S)-N-Cyclohexyl-3-{[(4-hydroxy-1-adamantyl)carbonyl]amino}piperidine-1-carboxamide

This compound was prepared using a procedure that was analogous to that described for the synthesis of example 1, steps 1-4. LCMS: (M+H)⁺=404.2.

### Example 5

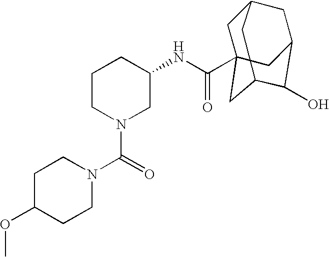

### 4-Hydroxy-N-{(3S)-1-[(4-methoxypiperidin-1-yl)carbonyl]piperidin-3-yl}adamantane-1-carboxamide

This compound was prepared using a procedure that was analogous to that described for the synthesis of example 1, steps 1-4. LCMS: (M+H)⁺=420.2.

### Example 6

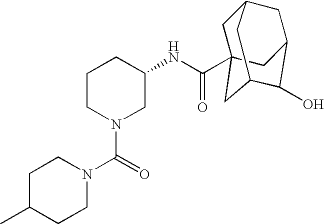

### 4-Hydroxy-N-{(3S)-1-[(4-methylpiperidin-1-yl)carbonyl]piperidin-3-yl}adamantane-1-carboxamide

This compound was prepared using a procedure that was analogous to that described for the synthesis of example 1, steps 1-4. LCMS: (M+H)⁺=404.2.

### Example 7

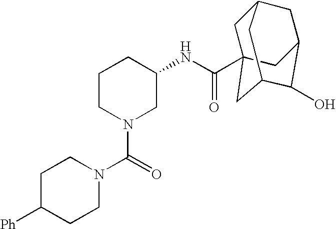

### 4-Hydroxy-N-{(3S)-1-[(4-phenylpiperidin-1-yl)carbonyl]piperidin-3-yl}adamantane-1-carboxamide

This compound was prepared using a procedure that was analogous to that described for the synthesis of example 1, steps 1-4. LCMS: (M+H)⁺=466.2.

### Example 8

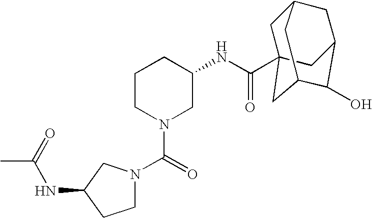

### N-((3S)-1-{[(3R)-3-(Acetylamino)pyrrolidin-1-yl]carbonyl}piperidin-3-yl)-4-hydroxyadamantane-1-carboxamide

Step 1. 4-nitrophenyl (3S)-3-{[(4-hydroxy-1-adamantyl)carbonyl]amino}piperidine-1-carboxylate

4-Hydroxy-N-[(3S)-piperidin-3-yl]adamantane-1-carboxamide (150 mg, 0.00054 mol, prepared by methods analogous to those described for the synthesis of example 1, steps 1-3) was dissolved in methylene chloride (3.8 mL, 0.060 mol) and triethylamine (0.15 mL, 0.0011 mol). To this solution was added p-nitrophenyl chloroformate (132 mg, 0.000654 mol). After stirring at rt for 4 h, the reaction mixture was washed with 0.1 N HCl twice and the combined aqueous layers were extracted with DCM. The combined organics were dried over MgSO₄, filtered, and the volatiles were removed in-vacuo to afford 691 mg of the desired product as a yellow solid. The 1H NMR spectra LCMS: (M+H)⁺=454.1/456.1. The product was used in the subsequent step without further purification.

Step 2. N-((3S)-1-{[(3R)-3-(acetylamino)pyrrolidin-1-yl]carbonyl}piperidin-3-yl)-4-hydroxyadamantane-1-carboxamide

To a solution of 4-nitrophenyl (3S)-3-{[(4-hydroxy-1-adamantyl)carbonyl]amino}piperidine-1-carboxylate (15 mg, 0.000034 mol) in tetrahydrofuran (0.5 mL, 0.006 mol) was added N-[(3R)-pyrrolidin-3-yl]acetamide (8.7 mg, 0.000068 mol) and N,N-diisopropylethylamine (18 uL, 0.00010 mol). After stirring the reaction mixture at room temperature for 2 h, the crude mixture was purified by prep-LCMS to afford the desired product. LCMS: (M+H)⁺=433.2.

### Example 9

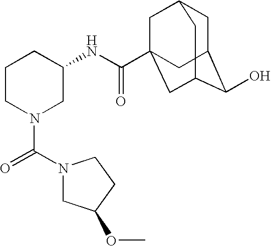

### 4-Hydroxy-N-((3S)-1-{[(3R)-3-methoxypyrrolidin-1-yl]carbonyl}piperidin-3-yl)adamantane-1-carboxamide

This compound was prepared using a procedure that was analogous to that described for the synthesis of example 8, steps 1 and 2. LCMS: (M+H)⁺=406.1.

### Example 10

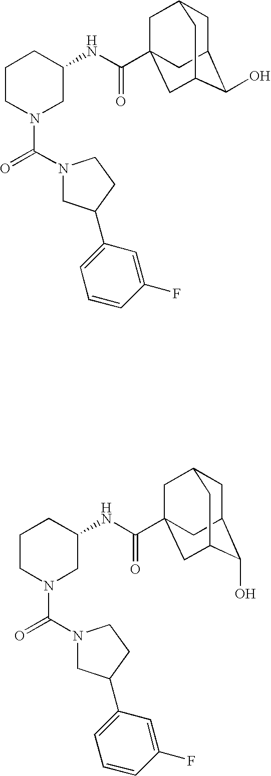

### N-((3S)-1-{[3-(3-Fluorophenyl)pyrrolidin-1-yl]carbonyl}piperidin-3-yl)-4-hydroxyadamantane-1-carboxamide

This compound was prepared using a procedure that was analogous to that described for the synthesis of example 8, steps 1-2. LCMS: (M+H)⁺=470.2.

### Example 11

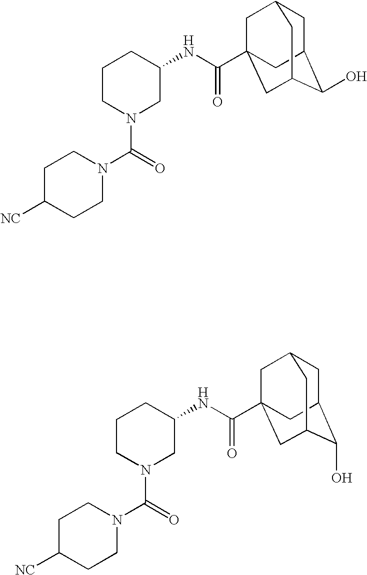

### N-{(3S)-1-[(4-Cyanopiperidin-1-yl)carbonyl]piperidin-3-yl}-4-hydroxyadamantane-1-carboxamide

This compound was prepared using a procedure that was analogous to that described for the synthesis of example 8, steps 1 and 2. LCMS: (M+H)⁺=415.3.

### Example 12

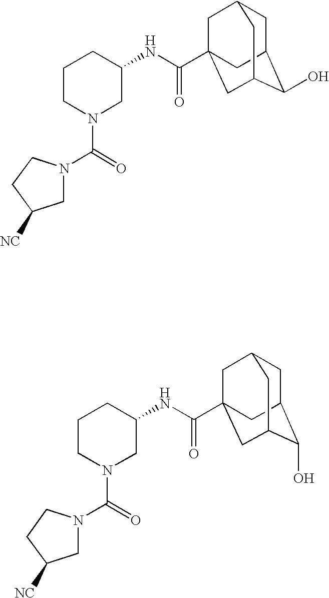

### N-((3S)-1-{[(3R)-3-Cyanopyrrolidin-1-yl]carbonyl}piperidin-3-yl)-4-hydroxyadamantane-1-carboxamide

This compound was prepared using a procedure that was analogous to that described for the synthesis of example 8, steps 1 and 2. LCMS: (M+H)⁺=401.3.

### Example 13

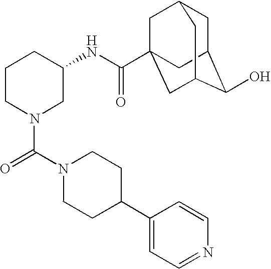

### 4-Hydroxy-N-{(3S)-1-[(4-pyridin-4-ylpiperidin-1-yl)carbonyl]piperidin-3-yl}adamantane-1-carboxamide

This compound was prepared using a procedure that was analogous to that described for the synthesis of example 8, steps 1 and 2. LCMS: (M+H)⁺=467.3.

### Example 14

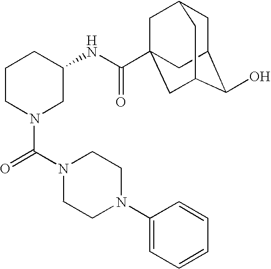

### 4-Hydroxy-N-{(3S)-1-[(4-phenylpiperazin-1-yl)carbonyl]piperidin-3-yl}adamantane-1-carboxamide

This compound was prepared using a procedure that was analogous to that described for the synthesis of example 8, steps 1 and 2. LCMS: (M+H)⁺=467.3.

### Example 15

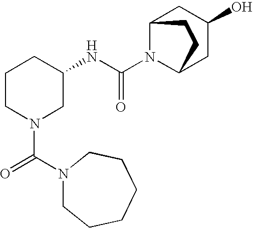

### (3-endo)-N-[(3S)-1-(Azepan-1-ylcarbonyl)piperidin-3-yl]-3-hydroxy-8-azabicyclo[3.2.1]octane-8-carboxamide

Step 1: tert-butyl (3-endo)-3-hydroxy-8-azabicyclo[3.2.1]octane-8-carboxylate

Boc-nortropinone (390 mg, 0.0017 mol) was dissolved in tetrahydrofuran (11 mL, 0.13 mol) and cooled to −69° C. (internal temperature). To this solution was added dropwise over 15 min. 1.0 M of diisobutylaluminum hydride in hexane (5.1 mL), while maintaining the temperature below −64° C. After stirring at this temperature for 3 h; the reaction was quenched with water. The reaction mixture was allowed to warm to −30° C. and water was added until effervescence ceased. The reaction mixture was then diluted with water and EtOAc and allowed to warm to ambient temperature. Sodium potassium tartrate (1 M) was added to break-up the clear gel. Following separation the organic layer was washed with sodium potassium tartrate (1 M), water, and brine. The combined organic layers were dried (Na2SO4), filtered, and the volatiles were removed to afford the desired axial alcohol product as a white solid. LCMS (M+Na)⁺=250.2.

Step 2: (3-endo)-8-Azabicyclo[3.2.1]octan-3-ol hydrochloride

tert-Butyl (3-endo)-3-hydroxy-8-azabicyclo[3.2.1]octane-8-carboxylate (195 mg, 0.000858 mol) was treated with 10 mL of 4 M HCl in dioxane at rt for 16 h. After removal of the volatiles in-vacuo, the desired HCl salt was isolated and used directly in the next step. LCMS (M+H)⁺=128.2.

Step 3: tert-butyl (3S)-3-{[(4-nitrophenoxy)carbonyl]amino}piperidine-1-carboxylate

To a mixture of p-nitrophenyl chloroformate (5.284 g, 0.02621 mol) and triethylamine (5.22 mL, 0.0374 mol) in methylene chloride (75.00 mL, 1.170 mol) at 0° C. was added a solution of tert-butyl (3S)-3-aminopiperidine-1-carboxylate (5.00 g, 0.0250 mol) in methylene chloride (25.00 mL, 0.3900 mol). After stirring at rt for 1 h, the reaction mixture was diluted with methylene chloride, washed with 1 N NaOH and brine, and the volatiles were removed in-vacuo to afford the desired product. The crude residue was used directly in the next step without further purification. LCMS (M+Na)⁺=388.2.

Step 4: tert-butyl (3S)-3-({[(3-endo)-3-hydroxy-8-azabicyclo[3.2.1]oct-8-yl]carbonyl}amino)piperidine-1-carboxylate

To a mixture of tert-butyl (3S)-3-{[(4-nitrophenoxy)carbonyl]amino}piperidine-1-carboxylate (4.91 g, 0.0134 mol) and (3-endo)-8-azabicyclo[3.2.1]octan-3-ol hydrochloride (2.00 g, 0.0122 mol) in acetonitrile (100.0 mL, 1.915 mol) was added triethylamine (5.11 mL, 0.0367 mol). After stirring at rt for 16 h, the reaction mixture was diluted with methylene chloride, washed with 1 N NaOH, brine, dried, and concentrated in-vacuo. The residue was purified on silica gel, eluting with 0 to 100% EtOAc in hexane, then 0 to 10% MeOH in methylene chloride, to give the desired product. LCMS (M+H)⁺=354.3.

Step 5: (3-endo)-3-hydroxy-N-[(3S)-piperidin-3-yl]-8-azabicyclo[3.2.1]octane-8-carboxamide hydrochloride

To a solution of tert-butyl (3S)-3-({[(3-endo)-3-hydroxy-8-azabicyclo[3.2.1]oct-8-yl]carbonyl}amino)piperidine-1-carboxylate (4.00 g, 0.0113 mol) in 10 mL of MeOH was added 40 mL of 4 M HCl in dioxane. The reaction mixture was stirred at rt for 16 h. The volatiles were removed in-vacuo and the crude solid was used directly in the next step. LCMS (M+H)⁺=254.3.

Step 6: (3-endo)-N-[(3S)-1-(azepan-1-ylcarbonyl)piperidin-3-yl]-3-hydroxy-8-azabicyclo[3.2.1]octane-8-carboxamide

To a mixture of p-nitrophenyl chloroformate (0.0230 g, 0.000114 mol) and triethylamine (0.0361 mL, 0.000259 mol) in acetonitrile (0.50 mL, 0.0096 mol) was added (3-endo)-3-hydroxy-N-[(3S)-piperidin-3-yl]-8-azabicyclo[3.2.1]octane-8-carboxamide hydrochloride (0.030 g, 0.00010 mol). After stirring at rt for 1 h, 1H-hexahydro-azepine, (0.0233 mL, 0.000207 mol) was added and the reaction mixture was heated at 100° C. and stirred for 16 h. The reaction mixture was allowed to cool to ambient temperature and was diluted with water. The crude product was purified by prep.-HPLC to afford the desired product. LCMS (M+H)⁺=379.3.

### Example 16

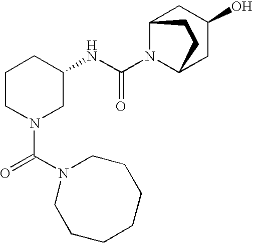

### (3-endo)-N-[(3S)-1-(Azocan-1-ylcarbonyl)piperidin-3-yl]-3-hydroxy-8-azabicyclo[3.2.1]octane-8-carboxamide

This compound was prepared using a procedure that was analogous to that described for the synthesis of example 15, steps 1-6. LCMS: (M+H)⁺=393.3.

### Example 17

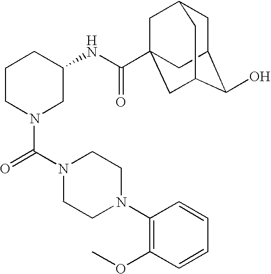

### 4-Hydroxy-N-((3S)-1-{[4-(2-methoxyphenyl)piperazin-1-yl]carbonyl}piperidin-3-yl)adamantane-1-carboxamide

Step 1: 4-hydroxy-N-[(3S)-1-(1H-imidazol-1-ylcarbonyl)piperidin-3-yl]adamantane-1-carboxamide

To a suspension of N,N-carbonyldiimidazole (0.38 g, 0.0024 mol) in tetrahydrofuran (4.0 mL, 0.049 mol) was added 4-hydroxy-N-[(3S)-piperidin-3-yl]adamantane-1-carboxamide (0.60 g, 0.0022 mol, prepared as the product in step 3 of example 1) and the resulting mixture was stirred at rt for 2 h. After removal of the volatiles in-vacuo, the resultant residue was dissolved in dichloromethane and washed with water (2×10 mL). The organic layer was dried over anhydrous MgSO4, filtered, and concentrated in vacuo to yield the desired product.

Step 2: 1-[((3S)-3-{[(4-hydroxy-1-adamantyl)carbonyl]amino}piperidin-1-yl)carbonyl]-3-methyl-1H-imidazol-3-ium iodide

To a solution of 4-hydroxy-N-[(3S)-1-(1H-imidazol-1-ylcarbonyl)piperidin-3-yl]adamantane-1-carboxamide (2.2 mmol, 0.0022 mol) in acetonitrile (5.0 mL, 0.096 mol) was added methyl iodide (550 uL, 0.0088 mol). The mixture was stirred at rt for 16 h. The solvent was removed under vacuum to yield the carbamoyl imidazolium salt, which was used in the next step without further purification.

Step 3: 4-hydroxy-N-((3S)-1-{[4-(2-methoxyphenyl)piperazin-1-yl]carbonyl}piperidin-3-yl)adamantane-1-carboxamide

To a solution of 1-[((3S)-3-{[(4-hydroxy-1-adamantyl)carbonyl]amino}piperidin-1-yl)carbonyl]-3-methyl-1H-imidazol-3-ium iodide (22.0 mg, 0.0000428 mol) in acetonitrile (0.5 mL, 0.01 mol) was added 1-(2-methoxyphenyl)piperazine (8.2 mg, 0.000043 mol) and triethylamine (12 uL, 0.000086 mol). The reaction mixture was stirred at rt for 16 h. The crude mixture was purified by prep.-LCMS to afford the desired product. LCMS: (M+H)⁺=497.3.

### Example 18

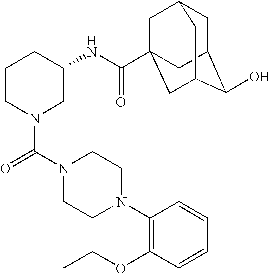

### N-((3S)-1-{[4-(2-Ethoxyphenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=511.3.

### Example 19

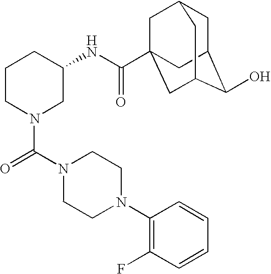

### N-((3S)-1-{[4-(2-Fluorophenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=485.3.

### Example 20

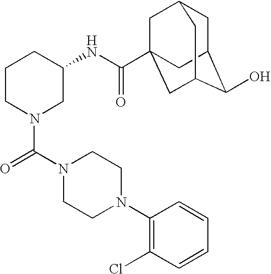

### N-((3S)-1-{[4-(2-Chlorophenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=501.3/503.3.

### Example 21

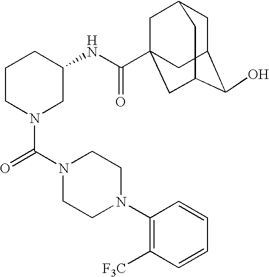

### 4-Hydroxy-N-[(3S)-1-({4-[2-(Trifluoromethyl)phenyl]piperazin-1-yl}carbonyl)piperidin-3-yl]adamantane-1-carboxamide

This compound was prepared using a procedure that was analogous to that described for the synthesis of example 17, steps 1-3. LCMS: (M+H)⁺=535.3.

### Example 22

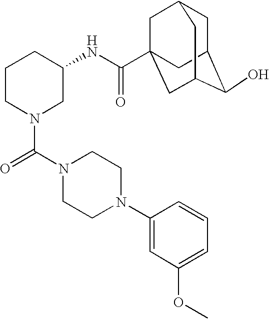

### 4-Hydroxy-N-((3S)-1-{[4-(3-methoxyphenyl)piperazin-1-yl]carbonyl}piperidin-3-yl)adamantane-1-carboxamide

This compound was prepared using a procedure that was analogous to that described for the synthesis of example 17, steps 1-3. LCMS: (M+H)⁺=497.3.

### Example 23

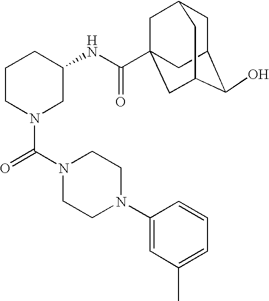

### 4-Hydroxy-N-((3S)-1-{[4-(3-methylphenyl)piperazin-1-yl]carbonyl}piperidin-3-yl)adamantane-1-carboxamide

This compound was prepared using a procedure that was analogous to that described for the synthesis of example 17, steps 1-3. LCMS: (M+H⁺=481.4.

### Example 24

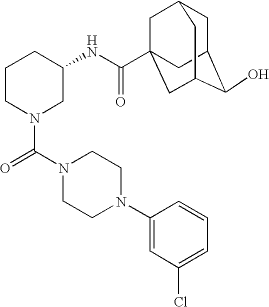

### N-((3S)-1-{[4-(3-Chlorophenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=501.3/503.3.

### Example 25

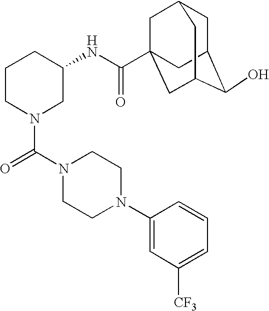

### 4-Hydroxy-N-[(3S)-1-({4-[3-(trifluoromethyl)phenyl]piperazin-1-yl}carbonyl)piperidin-3-yl]adamantane-1-carboxamide

This compound was prepared using a procedure that was analogous to that described for the synthesis of example 17, steps 1-3. LCMS: (M+H)⁺=535.3.

### Example 26

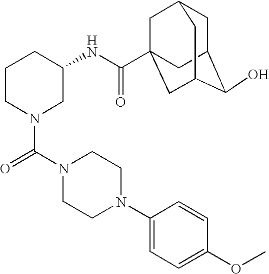

### 4-Hydroxy-N-((3S)-1-{[4-(4-methoxyphenyl)piperazin-1-yl]carbonyl}piperidin-3-yl)adamantane-1-carboxamide

This compound was prepared using a procedure that was analogous to that described for the synthesis of example 17, steps 1-3. LCMS: (M+H)⁺=497.3.

### Example 27

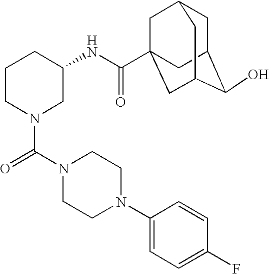

### N-((3S)-1-{[4-(4-Fluorophenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=485.3.

### Example 28

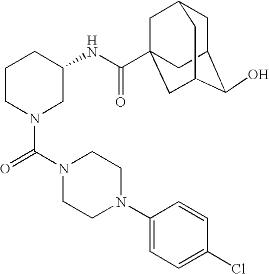

### N-((3S)-1-{[4-(4-Chlorophenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=501.3/ 503.3.

### Example 29

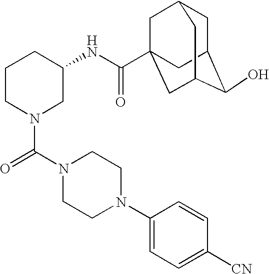

### N-((3S)-1-{[4-(4-Cyanophenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=492.3.

### Example 30

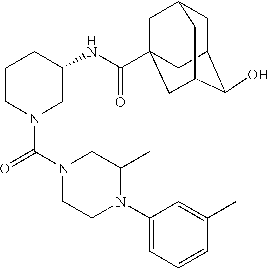

### 4-Hydroxy-N-((3S)-1-{[3-methyl-4-(3-methylphenyl)piperazin-1-yl]carbonyl}piperidin-3-yl)adamantane-1-carboxamide

This compound was prepared using a procedure that was analogous to that described for the synthesis of example 17, steps 1-3. LCMS: (M+H)⁺=495.3.

### Example 31

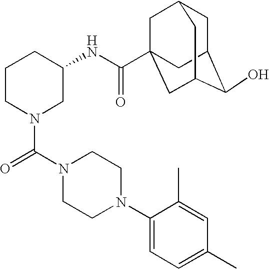

### N-((3S)-1-{[4-(2,4-Dimethylphenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=495.3.

### Example 32

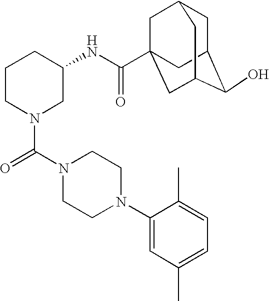

### N-((3S)-1-{[4-(2,5-dimethylphenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=495.3.

### Example 33

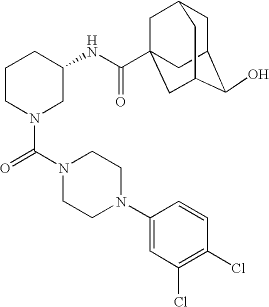

### N-((3S)-1-{[4-(3,4-Dichlorophenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=535.2/ 537.2.

### Example 34

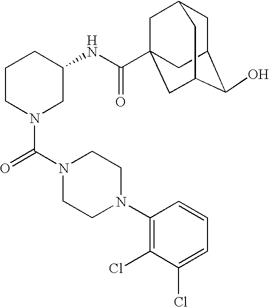

### N-((3S)-1-{[4-(2,4-Dimethylphenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=535.2/ 537.2.

### Example 35

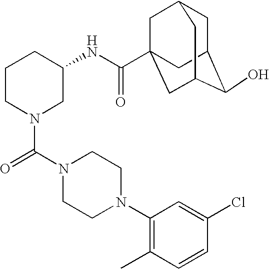

### N-((3S)-1-{[4-(5-Chloro-2-methylphenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=515.3/ 517.3.

### Example 36

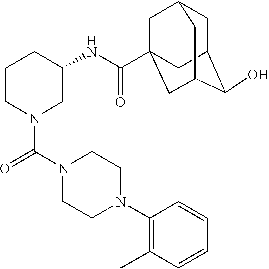

### 4-Hydroxy-N-((3S)-1-{[4-(2-methylphenyl)piperazin-1-yl]carbonyl}piperidin-3-yl)adamantane-1-carboxamide

This compound was prepared using a procedure that was analogous to that described for the synthesis of example 17, steps 1-3. LCMS: (M+H)⁺=481.3.

### Example 37

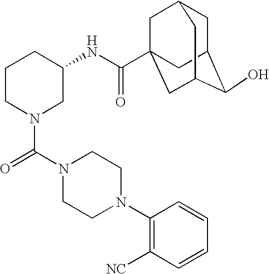

### N-((3S)-1-{[4-(2-Cyanophenyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=492.3.

### Example 38

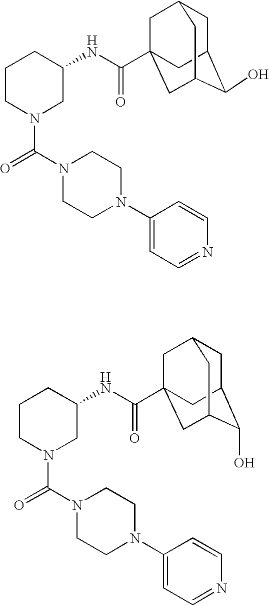

### 4-Hydroxy-N-{(3S)-1-[(4-pyridin-4-ylpiperazin-1-yl)carbonyl]piperidin-3-yl}adamantane-1-carboxamide

This compound was prepared using a procedure that was analogous to that described for the synthesis of example 17, steps 1-3. LCMS: (M+H)⁺=468.3.

### Example 39

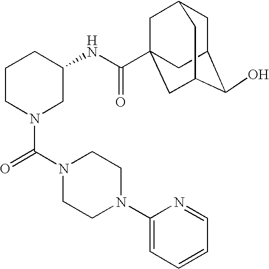

### 4-Hydroxy-N-{(3S)-1-[(4-pyridin-2-ylpiperazin-1-yl)carbonyl]piperidin-3-yl}adamantane-1-carboxamide

This compound was prepared using a procedure that was analogous to that described for the synthesis of example 17, steps 1-3. LCMS: (M+H)⁺=468.3.

### Example 40

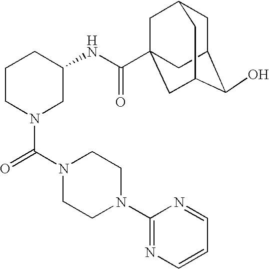

### 4-Hydroxy-N-{(3S)-1-[(4-pyrimidin-2-ylpiperazin-1-yl)carbonyl]piperidin-3-yl}adamantane-1-carboxamide

This compound was prepared using a procedure that was analogous to that described for the synthesis of example 17, steps 1-3. LCMS: (M+H)⁺=469.3.

### Example 41

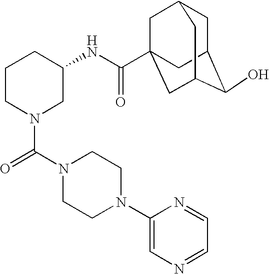

### 4-Hydroxy-N-{(3S)-1-[(4-pyrazin-2-ylpiperazin-1-yl)carbonyl]piperidin-3-yl}adamantane-1-carboxamide

This compound was prepared using a procedure that was analogous to that described for the synthesis of example 17, steps 1-3. LCMS: (M+H)⁺=469.3.

### Example 42

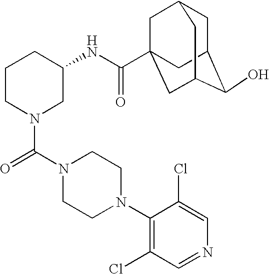

### N-((3S)-1-{[4-(3,5-Dichloropyridin-4-yl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=536.2/ 538.2.

### Example 43

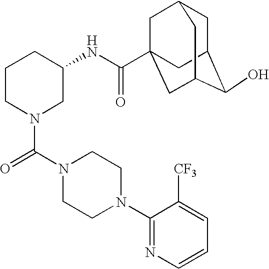

### 4-Hydroxy-N-[(3S)-1-({4-[3-(trifluoromethyl)pyridin-2-yl]piperazin-1-yl}carbony)piperidin-3-yl]adamantane-1-carboxamide

This compound was prepared using a procedure that was analogous to that described for the synthesis of example 17, steps 1-3. LCMS: (M+H)⁺=536.3.

### Example 44

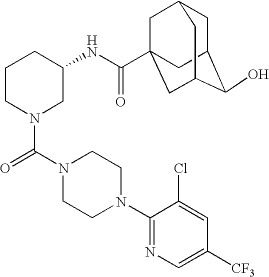

### N-[(3S)-1-({4-[3-Chloro-5-(trifluoromethyl)pyridin-2-yl]piperazin-1-yl}carbonyl)piperidin-3-yl]-4-hydroxyadamantane-1-carboxamide

This compound was prepared using a procedure that was analogous to that described for the synthesis of example 17, steps 1-3. LCMS: (M+H)⁺=570.2/572.2.

### Example 45

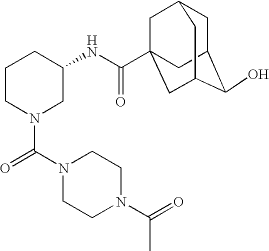

### N-{(3S)-1-[(4-Acetylpiperazin-1-yl)carbonyl]piperidin-3-yl}-4-hydroxyadamantane-1-carboxamide

This compound was prepared using a procedure that was analogous to that described for the synthesis of example 17, steps 1-3. LCMS: (M+H)⁺=433.3.

### Example 46

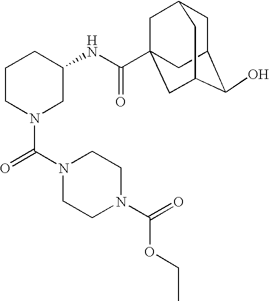

### Ethyl 4-[((3S)-3-{[(4-hydroxy-1-adamantyl)carbonyl]amino}piperidin-1-yl)carbonyl]piperazine-1-carboxylate

This compound was prepared using a procedure that was analogous to that described for the synthesis of example 17, steps 1-3. LCMS: (M+H)⁺=463.3.

### Example 47

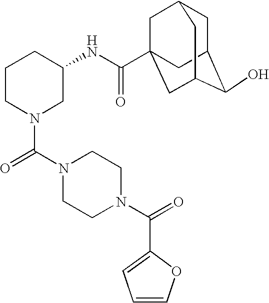

### N-((3S)-1-{[4-(2-Furoyl)piperazin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=485.3.

### Example 48

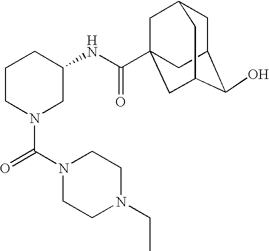

### N-{(3S)-1-[(4-Ethylpiperazin-1-yl)carbonyl]piperidin-3-yl}-4-hydroxyadamantane-1-carboxamide

This compound was prepared using a procedure that was analogous to that described for the synthesis of example 17, steps 1-3. LCMS: (M+H)⁺=419.3.

### Example 49

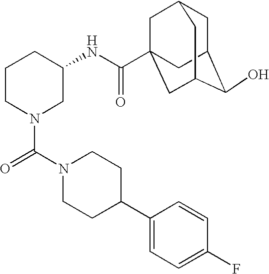

### N-((3S)-1-{[4-(4-Fluorophenyl)piperidin-1-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=484.3.

### Example 50

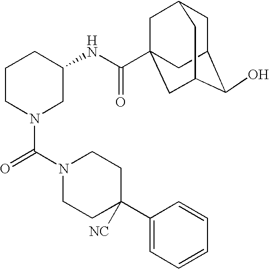

### N-{(3S)-1-[(4-Cyano-4-phenylpiperidin-1-yl)carbonyl]piperidin-3-yl}-4-hydroxyadamantane-1-carboxamide

This compound was prepared using a procedure that was analogous to that described for the synthesis of example 17, steps 1-3. LCMS: (M+H)⁺=491.3.

### Example 51

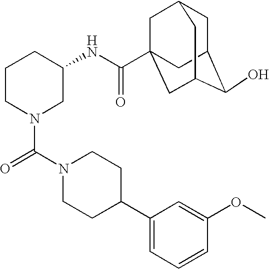

### 4-Hydroxy-N-((3S)-1-{[4-(3-methoxyphenyl)piperidin-1-yl]carbonyl}piperidin-3-yl)adamantane-1-carboxamide

This compound was prepared using a procedure that was analogous to that described for the synthesis of example 17, steps 1-3. LCMS: (M+H)⁺=496.3.

### Example 52

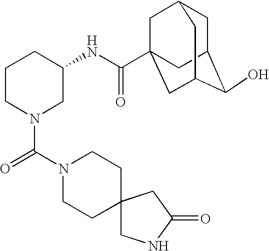

### 4-Hydroxy-N-{(3S)-1-[(3-oxo-2,8-diazaspiro[4.5]dec-8-yl)carbonyl]piperidin-3-yl}adamantane-1-carboxamide

This compound was prepared using a procedure that was analogous to that described for the synthesis of example 17, steps 1-3. LCMS: (M+H)⁺=459.3.

### Example 53

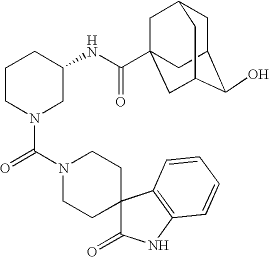

### 4-Hydroxy-N-{(3S)-1-[(2-oxo-1,2-dihydro-1′H-spiro[indole-3,4′-piperidin]-1′-yl)carbonyl]piperidin-3-yl}adamantane-1-carboxamide

This compound was prepared using a procedure that was analogous to that described for the synthesis of example 17, steps 1-3. LCMS: (M+H)⁺=507.3.

### Example 54

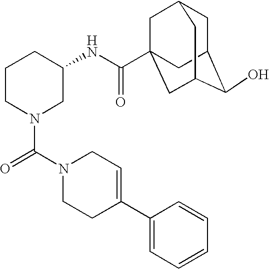

### 4-Hydroxy-N-{(3S)-1-[(4-phenyl-3,6-dihydropyridin-1(2H)-yl)carbonyl]piperidin-3-yl}adamantane-1-carboxamide

This compound was prepared using a procedure that was analogous to that described for the synthesis of example 17, steps 1-3. LCMS: (M+H)⁺=464.3.

### Example 55

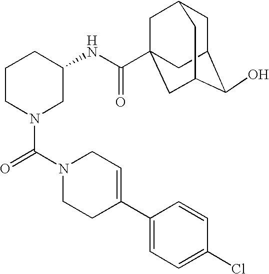

### N-((3S)-1-{[4-(4-Chlorophenyl)-3,6-dihydropyridin-1(2H)-yl]carbonyl}piperidin-3-yl)-4-hydroxyadamantane-1-carboxamide

This compound was prepared using a procedure that was analogous to that described for the synthesis of example 17, steps 1-3. LCMS: (M+H)⁺=498.3/500.3.

### Example 56

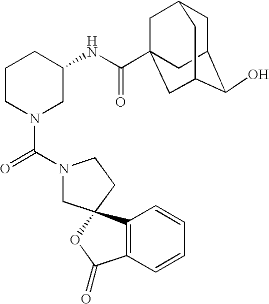

### 4-Hydroxy-N-((3S)-1-{[(1R)-3-oxo-1′H,3H-spiro[2-benzofuran-1,3′-pyrrolidin]-1′-yl]carbonyl}piperidin-3-yl)adamantane-1-carboxamide

This compound was prepared using a procedure that was analogous to that described for the synthesis of example 17, steps 1-3. LCMS: (M+H)⁺=495.3. Synthesis of (IS)-(+)-10-camphorsulfonic acid-3H-spiro-[2-benzofuran-1,3′-pyrrolidin]-3-one (used in step 3) is provided as follows.

Synthesis of (1S)-(+)-10-Camphorsulfonic acid-3H-spiro-[2-benzofuran-1,3′-pyrrolidin]-3-one.

Step 1. Benzyl 3-oxo-1′H,3H-spiro[2-benzofuran-1,3′-pyrrolidine]-1′carboxylate

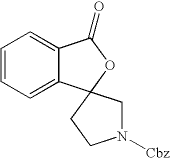

To a solution of methyl-2-iodobenzoate (8.8 mL, 0.060 mol) in THF (300 mL) at −60° C. was slowly added a solution of isopropylmagnesium bromide in THF (1.0 M, 66.0 mL) and the mixture was stirred below −50° C. for 1 h. A solution of benzyl-3-oxopyrrolidine-1-carboxylate (11.0 g, 0.05 mol) in THF (20.0 mL) was added to the above mixture and the reaction mixture was stirred below −20° C. for 2 h. The reaction was quenched by the addition of saturated NH₄Cl aqueous solution and the resulting mixture was extracted with ethyl acetate several times. The combined extracts were washed with water and brine, dried, and concentrated in-vacuo. The product was purified by CombiFlash eluting with hexane/ethyl acetate.
Step 2. (IS)-(+)-10-Camphorsulfonic acid-3H-spiro-[2-benzofuran-1,3′-pyrrolidin]-3-one

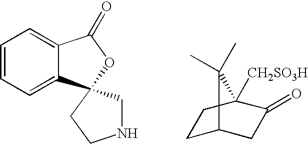

Palladium on carbon (10%, 0.5 g) was added to a solution of benzyl 3-oxo-1′H,3H-spiro[2-benzofuran-1,3′-pyrrolidine]-1′carboxylate (5.0 g, 15.5 mmol) in methanol (100 mL) and the mixture was stirred under hydrogen balloon for 4 h. The volatiles were removed under reduced pressure and the residue was dissolved in acetonitrile (200 mL). The solution was heated to 50° C. prior to the slow addition of a solution of (1S)-(+)-10-camphorsulfonic acid (3.6 g, 15.5 mmol) in acetonitrile (20 mL). The crystalline solid that was formed was filtered and dried to afford the desired product. LC-MS 190.1 (M+H)⁺.

### Example 57

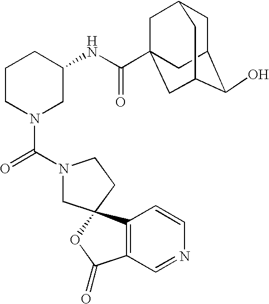

### 4-Hydroxy-N-((3S)-1-{[(1R)-3-oxo-1′H,3H-spiro[furo[3,4-c]pyridine-1,3′-pyrrolidin]-1′-yl]carbonyl}piperidin-3-yl)adamantane-1-carboxamide

This compound was prepared using a procedure that was analogous to that described for the synthesis of example 56. LCMS: (M+H)⁺=495.3.

### Example 58

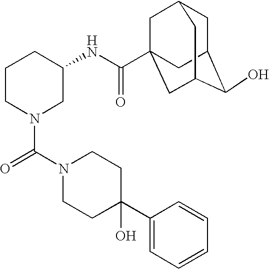

### 4-Hydroxy-N-{(3S)-1-[(4-hydroxy-4-phenylpiperidin-1-yl)carbonyl]piperidin-3-yl}adamantane-1-carboxamide

This compound was prepared using a procedure that was analogous to that described for the synthesis of example 17, steps 1-3. LCMS: (M+H)⁺=482.3.

### Example 59

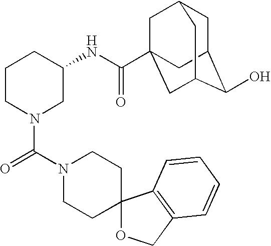

### 4-Hydroxy-N-[(3S)-1-(1′H,3H-spiro[2-benzofuran-1,4′-piperidin]-1′-ylcarbonyl)piperidin-3-yl]adamantane-1-carboxamide

This compound was prepared using a procedure that was analogous to that described for the synthesis of example 17, steps 1-3. LCMS: (M+H)⁺=494.3.

### Example 60

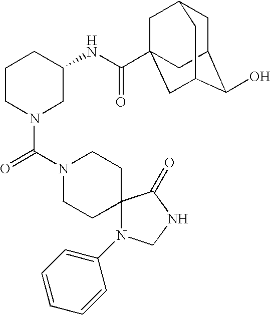

### 4-Hydroxy-N-{(3S)-1-[(4-oxo-1-phenyl-1,3,8-triazaspiro[4.5]dec-8-yl)carbonyl]piperidin-3-yl}adamantane-1-carboxamide

This compound was prepared using a procedure that was analogous to that described for the synthesis of example 17, steps 1-3. LCMS: (M+H)⁺=536.3.

### Example 61

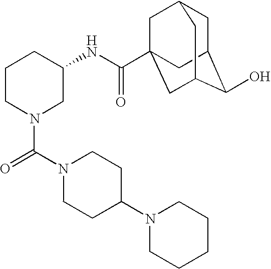

### N-[(3S)-1-(1,4′-Bipiperidin-1′-ylcarbonyl)piperidin-3-yl]-4-hydroxyadamantane-1-carboxamide

This compound was prepared using a procedure that was analogous to that described for the synthesis of example 17, steps 1-3. LCMS: (M+H)⁺=473.3.

### Example 62

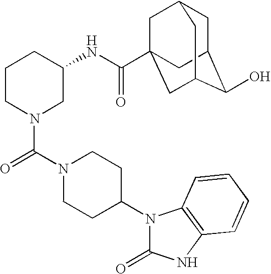

### 4-Hydroxy-N-((3S)-1-{[4-(2-oxo-2,3-dihydro-1H-benzimidazol-1-yl)piperidin-1-yl]carbonyl}piperidin-3-yl)adamantane-1-carboxamide

This compound was prepared using a procedure that was analogous to that described for the synthesis of example 17, steps 1-3. LCMS: (M+H)⁺=522.3.

### Example 63

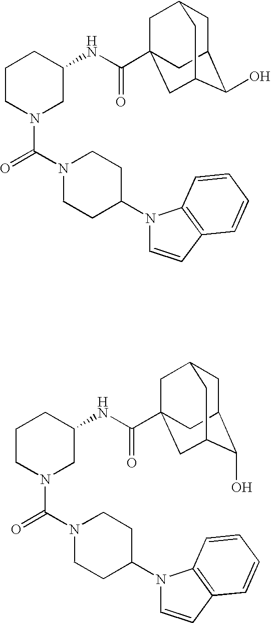

### 4-Hydroxy-N-((3S)-1-{[4-(1H-indol-1-yl)piperidin-1-yl]carbonyl}piperidin-3-yl)adamantane-1-carboxamide

This compound was prepared using a procedure that was analogous to that described for the synthesis of example 17, steps 1-3. LCMS: (M+H)⁺=505.3.

### Example 64

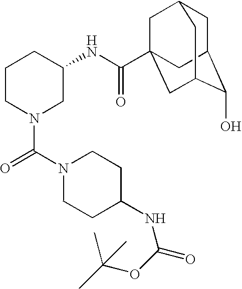

### tert-Butyl {1-[((3S)-3-{[(4-hydroxy-1-adamantyl)carbonyl]amino}piperidin-1-yl)carbonyl]piperidin-4-yl}carbamate

This compound was prepared using a procedure that was analogous to that described for the synthesis of example 17, steps 1-3. LCMS: (M+H)⁺=505.3.

### Example 65

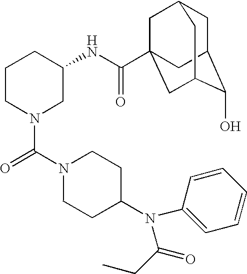

### 4-Hydroxy-N-[(3S)-1-({4-[phenyl(propionyl)amino]piperidin-1-yl}carbonyl)piperidin-3-yl]adamantane-1-carboxamide

This compound was prepared using a procedure that was analogous to that described for the synthesis of example 17, steps 1-3. LCMS: (M+H)⁺=537.4.

### Example 66

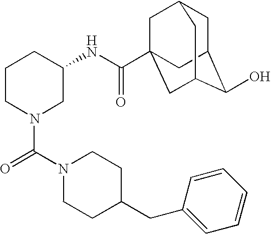

### N-{(3S)-1-[(4-Benzylpiperidin-1-yl)carbonyl]piperidin-3-yl}-4-hydroxyadamantane-1-carboxamide

This compound was prepared using a procedure that was analogous to that described for the synthesis of example 17, steps 1-3. LCMS: (M+H)⁺=480.3.

### Example 67

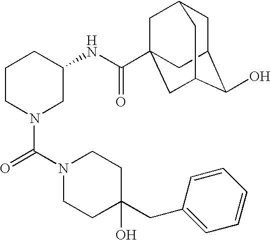

### N-{(3S)-1-[(4-Benzyl-4-hydroxypiperidin-1-yl)carbonyl]piperidin-3-yl}-4-hydroxyadamantane-1-carboxamide

This compound was prepared using a procedure that was analogous to that described for the synthesis of example 17, steps 1-3. LCMS: (M+H)⁺=496.3.

### Example 68

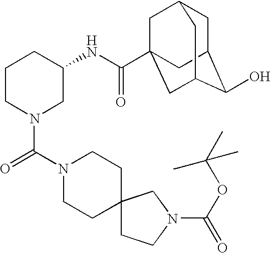

### tert-Butyl 8-[((3S)-3-{[(4-hydroxy-1-adamantyl)carbonyl]amino}piperidin-1-yl)carbonyl]-2,8-diazaspiro[4.5]decane-2-carboxylate

This compound was prepared using a procedure that was analogous to that described for the synthesis of example 17, steps 1-3. LCMS: (M+H)⁺=545.4.

### Example 69

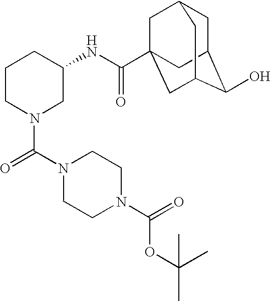

### tert-Butyl 4-[((3S)-3-{[(4-hydroxy-1-adamantyl)carbonyl]amino}piperidin-1-yl)carbonyl]piperazine-1-carboxylate

This compound was prepared using a procedure that was analogous to that described for the synthesis of example 17, steps 1-3. LCMS: (M+H)⁺=491.3.

### Example 70

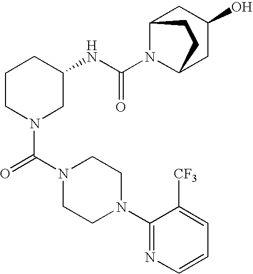

### (3-endo)-3-Hydroxy-N-[(3S)-1-({4-[3-(trifluoromethyl)pyridin-2-yl]piperazin-1-yl}carbonyl)piperidin-3-yl]-8-azabicyclo[3.2.1]octane-8-carboxamide

This compound was prepared using a procedure that was analogous to that described for the synthesis of example 15, steps 1-6. LCMS: (M+H)⁺=511.2.

### Example 71

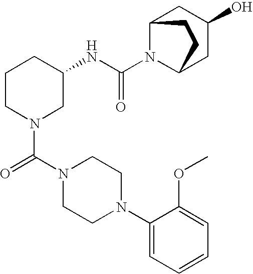

### (3-endo)-3-Hydroxy-N-((3S)-1-{[4-(2-methoxyphenyl)piperazin-1-yl]carbonyl}piperidin-3-yl)-8-azabicyclo[3.2.1]octane-8-carboxamide

This compound was prepared using a procedure that was analogous to that described for the synthesis of example 15, steps 1-6. LCMS: (M+H)⁺=472.3.

### Example 72

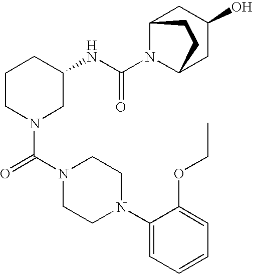

### (3-endo)-N-((3S)-1-{[4-(2-Ethoxyphenyl)piperazin-1-yl]carbonyl}piperidin-3-yl)-3-hydroxy-8-azabicyclo[3.2.1]octane-8-carboxamide

This compound was prepared using a procedure that was analogous to that described for the synthesis of example 15, steps 1-6. LCMS: (M+H)⁺=486.3.

### Example 73

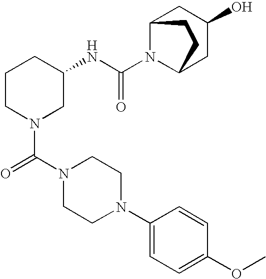

### (3-endo)-3-Hydroxy-N-((3S)-1-{[4-(4-methoxyphenyl)piperazin-1-yl]carbonyl}piperidin-3-yl)-8-azabicyclo[3.2.1]octane-8-carboxamide

This compound was prepared using a procedure that was analogous to that described for the synthesis of example 15, steps 1-6. LCMS: (M+H)⁺=472.3.

### Example 74

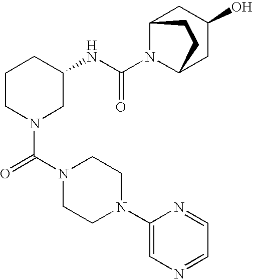

### (3-endo)-3-Hydroxy-N-{(3S)-1-[(4-pyrazin-2-ylpiperazin-1-yl)carbonyl]piperidin-3-yl}-8-azabicyclo[3.2.1]octane-8-carboxamide

This compound was prepared using a procedure that was analogous to that described for the synthesis of example 15, steps 1-6. LCMS: (M+H)⁺=444.3.

### Example 75

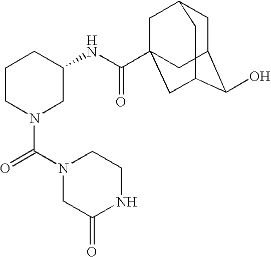

### 4-Hydroxy-N-{(3S)-1-[(3-oxopiperazin-1-yl)carbonyl]piperidin-3-yl}adamantane-1-carboxamide

This compound was prepared using a procedure that was analogous to that described for the synthesis of example 17, steps 1-3. LCMS: (M+H)⁺=405.3.

### Example 76

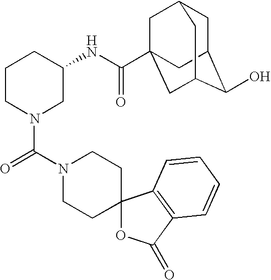

### 4-Hydroxy-N-{(3S)-1-[(3-oxo-1′H,3H-spiro[2-benzofuran-1,4′-piperidin]-1′-yl)carbonyl]piperidin-3-yl}adamantane-1-carboxamide

This compound was prepared using a procedure that was analogous to that described for the synthesis of example 56. LCMS: (M+H)⁺=508.3.

### Example 77

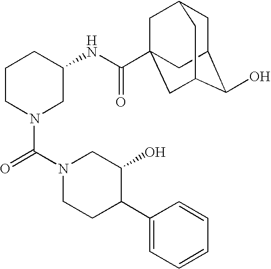

### 4-Hydroxy-N-((3S)-1-{[(3R,4R)-3-hydroxy-4-phenylpiperidin-1-yl]carbonyl}piperidin-3-yl)adamantane-1-carboxamide

This compound was prepared using a procedure that was analogous to that described for the synthesis of example 17, steps 1-3. LCMS: (M+H)⁺=482.3.

### Example 78

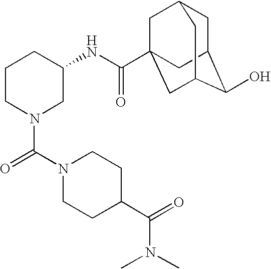

### 1-[((3S)-3-{[(4-Hydroxy-1-adamantyl)carbonyl]amino}piperidin-1-yl)carbonyl]-N,N-dimethylpiperidine-4-carboxamide

This compound was prepared using a procedure that was analogous to that described for the synthesis of example 17, steps 1-3. LCMS: (M+H)⁺=461.3.

### Example 79

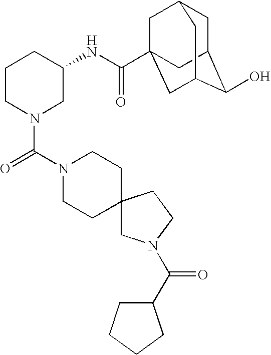

### N-((3S)-1-{[2-(Cyclopentylcarbonyl)-2,8-diazaspiro[4.5]dec-8-yl]carbonyl}piperidin-3-yl)-4-hydroxyadamantane-1-carboxamide

Step 1. N-[(3S)-1-(2,8-diazaspiro[4.5]dec-8-ylcarbonyl)piperidin-3-yl]-4-hydroxyadamantane-1-carboxamide hydrochloride

4.0 M HCl in dioxane (1 mL) was added to tert-butyl 8-[((3S)-3-{[(4-hydroxy-1-adamantyl)carbonyl]amino}piperidin-1-yl)carbonyl]-2,8-diazaspiro[4.5]decane-2-carboxylate (20 mg, 0.00004 mol, this compound was prepared using a procedure that was analogous to that described for the synthesis of example 17, steps 1-3). The reaction mixture was stirred at room temperature for 2 h. The volatiles were removed in-vacuo to afford the desired product, which was used directly in the next step.

Step 2. N-((3S)-1-{[2-(cyclopentylcarbonyl)-2,8-diazaspiro[4.5]dec-8-yl]carbonyl}piperidin-3-yl)-4-hydroxyadamantane-1-carboxamide

Cyclopentanecarbonyl chloride (5.0 uL, 0.000042 mol) was added to a solution of N-[(3S)-1-(2,8-diazaspiro[4.5]dec-8-ylcarbonyl)piperidin-3-yl]4-hydroxyadamantane-1-carboxamide hydrochloride (10 mg, 0.00002 mol) and N,N-diisopropylethylamine (11 uL, 0.000062 mol) in acetonitrile (0.5 mL, 0.01 mol). The crude reaction mixture was purified by prep.-LCMS to afford the desired product. LCMS: (M+H)⁺=541.4.

### Example 80

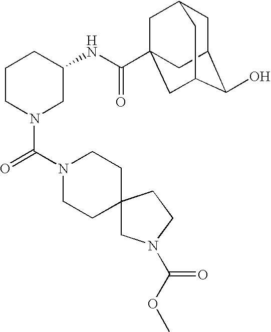

### Methyl 8-[((3S)-3-{[(4-hydroxy-1-adamantyl)carbonyl]amino}piperidin-1-yl)carbonyl]-2,8-diazaspiro[4.5]decane-2-carboxylate

This compound was prepared using a procedure that was analogous to that described for the synthesis of example 79, steps 1 and 2. LCMS: (M+H)⁺=503.3.

### Example 81

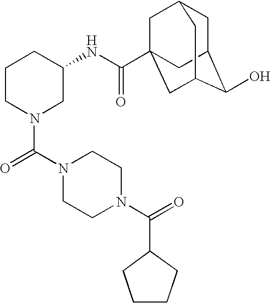

### N-((3S)-1-{[4-(Cyclopentylcarbonyl)piperazin-1-yl]carbonyl}piperidin-3-yl)-4-hydroxyadamantane-1-carboxamide

This compound was prepared using a procedure that was analogous to that described for the synthesis of example 79, steps 1 and 2. LCMS: (M+H)⁺=487.4.

### Example 82

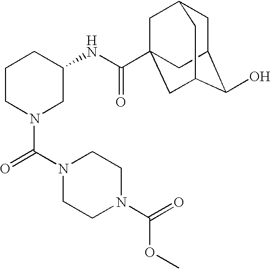

### Methyl 4-[((3S)-3-{[(4-hydroxy-1-adamantyl)carbonyl]amino}piperidin-1-yl)carbonyl]piperazine-1-carboxylate

This compound was prepared using a procedure that was analogous to that described for the synthesis of example 79, steps 1 and 2. LCMS: (M+H)⁺=449.3.

### Example 83

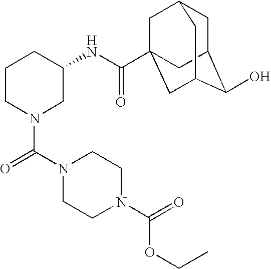

### Ethyl 4-[((3S)-3-{[(4-hydroxy-1-adamantyl)carbonyl]amino}piperidin-1-yl)carbonyl]piperazine-1-carboxylate

This compound was prepared using a procedure that was analogous to that described for the synthesis of example 17, steps 1-3. LCMS: (M+H)⁺=463.3.

### Example 84

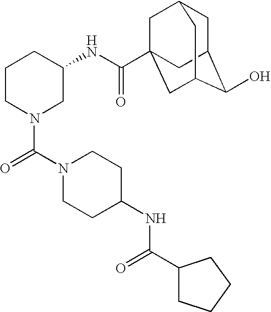

### N-[(3S)-1-({4-[(Cyclopentylcarbonyl)amino]piperidin-1-yl}carbonyl)piperidin-3-yl]-4-hydroxyadamantane-1-carboxamide

This compound was prepared using a procedure that was analogous to that described for the synthesis of example 79, steps 1 and 2. LCMS: (M+H)⁺=501.4.

### Example 85

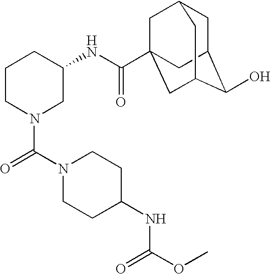

### Methyl {1-[((3S)-3-{[(4-hydroxy-1-adamantyl)carbonyl]amino}piperidin-1-yl)carbonyl]piperidin-4-yl}carbamate

This compound was prepared using a procedure that was analogous to that described for the synthesis of example 79, steps 1 and 2. LCMS: (M+H)⁺=463.3.

### Example 86

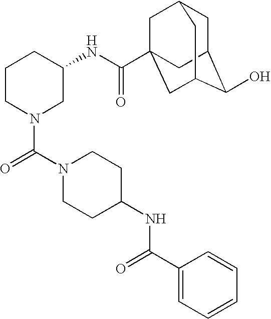

### N-((3S)-1-{[4-(Benzoylamino)piperidin-1-yl]carbonyl}piperidin-3-yl)-4-hydroxyadamantane-1-carboxamide

This compound was prepared using a procedure that was analogous to that described for the synthesis of example 79, steps 1 and 2. LCMS: (M+H)⁺=509.4.

### Example 87

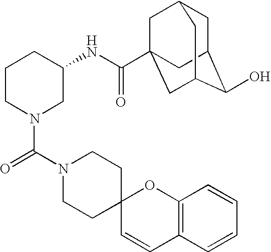

### 4-Hydroxy-N-[(3S)-1-(1′H-spiro[chromene-2,4′-piperidin]-1′-ylcarbonyl)piperidin-3-yl]adamantane-1-carboxamide

This compound was prepared using a procedure that was analogous to that described for the synthesis of example 17, steps 1-3. LCMS: (M+H)⁺=506.3.

### Example A

### Enzymatic Assay of 11βHSD1

All in vitro assays were performed with clarified lysates as the source of 11βHSD1 activity. HEK-293 transient transfectants expressing an epitope-tagged version of full-length human 11βHSD1 were harvested by centrifugation. Roughly 2×10⁷ cells were resuspended in 40 mL of lysis buffer (25 mM Tris-HCl, pH 7.5, 0.1 M NaCl, 1 mM MgCl₂ and 250 mM sucrose) and lysed in a microfluidizer. Lysates were clarified by centrifugation and the supernatants were aliquoted and frozen.

Inhibition of 11βHSD1 by test compounds was assessed in vitro by a Scintillation Proximity Assay (SPA). Dry test compounds were dissolved at 5 mM in DMSO. These were diluted in DMSO to suitable concentrations for the SPA assay. 0.8 μL of 2-fold serial dilutions of compounds were dotted on 384 well plates in DMSO such that 3 logs of compound concentration were covered. 20 μL of clarified lysate was added to each well. Reactions were initiated by addition of 20 μL of substrate-cofactor mix in assay buffer (25 mM Tris-HCl, pH 7.5, 0.1 M NaCl, 1 mM MgCl₂) to final concentrations of 400 μM NADPH, 25 nM ³H-cortisone and 0.007% Triton X-100. Plates were incubated at 37° C. for one hour. Reactions were quenched by addition of 40 μL of anti-mouse coated SPA beads that had been pre-incubated with 10 μM carbenoxolone and a cortisol-specific monoclonal antibody. Quenched plates were incubated for a minimum of 30 minutes at RT prior to reading on a Topcount scintillation counter. Controls with no lysate, inhibited lysate, and with no mAb were run routinely. Roughly 30% of input cortisone is reduced by 11βHSD1 in the uninhibited reaction under these conditions.

Test compounds having an IC₅₀ value less than about 20 μM according to this assay were considered active.

### Example B

### Cell-Based Assays for HSD Activity

Peripheral blood mononuclear cells (PBMCs) were isolated from normal human volunteers by Ficoll density centrifugation. Cells were plated at 4×10⁵ cells/well in 200 μL of AIM V (Gibco-BRL) media in 96 well plates. The cells were stimulated overnight with 50 ng/ml recombinant human IL-4 (R&D Systems). The following morning, 200 nM cortisone (Sigma) was added in the presence or absence of various concentrations of compound. The cells were incubated for 48 hours and then supernatants were harvested. Conversion of cortisone to cortisol was determined by a commercially available ELISA (Assay Design).

Test compounds having an IC₅₀ value less than about 20 μM according to this assay were considered active.

### Example C

### Cellular Assay to Evaluate MR Antagonism

Assays for MR antagonism were performed essentially as described (Jausons-Loffreda et al. J Biolumin and Chemilumin, 1994, 9: 217-221). Briefly, HEK293/MSR cells (Invitrogen Corp.) were co-transfected with three plasmids: 1) one designed to express a fusion protein of the GAL4 DNA binding domain and the mineralocorticoid receptor ligand binding domain, 2) one containing the GAL4 upstream activation sequence positioned upstream of a firefly luciferase reporter gene (pFR-LUC, Stratagene, Inc.), and 3) one containing the Renilla luciferase reporter gene cloned downstream of a thymidine kinase promoter (Promega). Transfections were performed using the FuGENE6 reagent (Roche). Transfected cells were ready for use in subsequent assays 24 hours post-transfection.

In order to evaluate a compound's ability to antagonize the MR, test compounds are diluted in cell culture medium (E-MEM, 10% charcoal-stripped FBS, 2 mM L-glutamine) supplemented with 1 nM aldosterone and applied to the transfected cells for 16-18 hours. After the incubation of the cells with the test compound and aldosterone, the activity of firefly luciferase (indicative of MR agonism by aldosterone) and Renilla luciferase (normalization control) were determined using the Dual-Glo Luciferae Assay System (Promega). Antagonism of the mineralocorticoid receptor was determined by monitoring the ability of a test compound to attenuate the aldosterone-induced firefly luciferase activity.

Compounds having an IC₅₀ of 100 μM or less were considered active.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula I 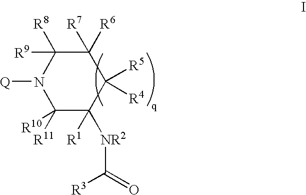
or a pharmaceutically acceptable salt or prodrug thereof, wherein:
Q is —SO₂-Cy, —C(O)O-Cy or —C(O)NR^{A}R^{B};
Cy is cycloalkyl or heterocycloalkyl, each optionally substituted by 1, 2, 3, 4 or 5 —W—X—Y-Z;
R^{A} and R^{B} are independently selected from H, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl, and heterocycloalkylalkyl, wherein said C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl, or heterocycloalkylalkyl are each optionally substituted with 1, 2, 3, 4 or 5 —W—X—Y-Z;
or R^{A} and R^{B} together with the N atom to which they are attached form a 4-20 membered heterocycloalkyl ring optionally substituted with 1, 2, 3, 4 or 5 —W—X—Y-Z;
R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;
R² is H, C₁₋₆ alkyl, arylalkyl, heteroarylalkyl, cycloalkyl, cycloalkylalkyl, heterocycloalkyl or heterocycloalkylalkyl, wherein said C₁₋₆ alkyl, arylalkyl, heteroarylalkyl, cycloalkyl, cycloalkylalkyl, heterocycloalkyl or heterocycloalkylalkyl is optionally substituted by 1, 2 or 3 R¹⁴;
R³ is H, NR^{3a}R^{3b}, C₁₋₆ alkyl, aryl, cycloalkyl, heteroaryl, or heterocycloalkyl, wherein said C₁₋₆ alkyl, aryl, cycloalkyl, heteroaryl, or heterocycloalkyl is optionally substituted by 1, 2 or 3 —W′—X′—Y′-Z′;
R^{3a} and R^{3b} are independently selected from H, C₁₋₆ alkyl, aryl, cycloalkyl, heteroaryl, and heterocycloalkyl, wherein said C₁₋₆ alkyl, aryl, cycloalkyl, heteroaryl, or heterocycloalkyl is optionally substituted by 1, 2 or 3 —W′—X′—Y′-Z′;
or R^{3a} and R^{3b} together with the N atom to which they are attached form a 4-14 membered heterocycloalkyl group which is optionally substituted by 1, 2 or 3 —W′—X′—Y′-Z′;
R⁴, R⁵, R⁶, R⁷, R⁸, R⁹, R¹⁰ and R¹¹ are independently selected from H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl is optionally substituted by 1, 2 or 3 R¹⁴;
or R² and R³ together with the nitrogen and carbon atoms to which they are attached form a 3-14 membered heterocycloalkyl group which is optionally substituted by 1, 2 or 3 R¹⁴;
or R⁴ and R⁵ together with the carbon atom to which they are attached form a 3-14 membered cycloalkyl or 3-14 membered heterocycloalkyl group which is optionally substituted by 1, 2 or 3 R¹⁴;
or R⁶ and R⁷ together with the carbon atom to which they are attached form a 3-14 membered cycloalkyl or 3-14 membered heterocycloalkyl group which is optionally substituted by 1, 2 or 3 R¹⁴;
or R⁸ and R⁹ together with the carbon atom to which they are attached form a 3-14 membered cycloalkyl or 3-14 membered heterocycloalkyl group which is optionally substituted by 1, 2 or 3 R¹⁴;
or R¹⁰ and R¹¹ together with the carbon atom to which they are attached form a 3-14 membered cycloalkyl or 3-14 membered heterocycloalkyl group which is optionally substituted by 1, 2 or 3 R¹⁴;
or R⁴ and R⁶ together with the carbon atoms to which they are attached form a 3-7 membered fused cycloalkyl group or 3-7 membered fused heterocycloalkyl group which is optionally substituted by 1, 2 or 3 R¹⁴;
or R⁶ and R⁸ together with the carbon atoms to which they are attached form a 3-7 membered fused cycloalkyl group or 3-7 membered fused heterocycloalkyl group which is optionally substituted by 1, 2 or 3 R¹⁴;
R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of said C₁₋₆ alkylenyl, C₂₋₆ alkenylenyl, or C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino, and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl, and heterocycloalkyl, wherein each of said C₁₋₆ alkylenyl, C₂₋₆ alkenylenyl, C₂₋₆ alkynylenyl, cycloalkyl, heteroaryl or heterocycloalkyl is optionally substituted by one or more substituents independently selected from halo, CN, NO₂, OH, C₁₋₄ alkoxy, C₁₋₄ haloalkoxy, amino, C₁₋₄ alkylamino, and C₂₋₈ dialkylamino;
Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein each of said C₁₋₆ alkylenyl, C₂₋₆ alkenylenyl, and C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino, and C₂₋₈ dialkylamino;
Z, Z′ and Z″ are independently selected from H, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, pentahalosulfanyl, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl, and heterocycloalkyl, wherein each of said C₁₋₆ alkyl, C₂₋₆ alkenyl, C₂₋₆ alkynyl, aryl, cycloalkyl, heteroaryl or heterocycloalkyl is optionally substituted by 1, 2 or 3 substituents independently selected from halo, C₁₋₆ alkyl, C₂₋₆ alkenyl, C₂₋₆ alkynyl, C₁₋₄ haloalkyl, aryl, cycloalkyl, heteroaryl, heterocycloalkyl, pentahalosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkyl or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{e} and R^{f} together with the N atom to which they are attached form a 4-, 5-, 6- or 7-membered heterocycloalkyl group; and
q is 1 or 2.
with the provisos:
(a) when q is 1 and R⁴ is H, then R⁵ is other than —NHC(O)R^{g}, wherein R^{g} is heteroaryl substituted by halo;
(b) when Q is —C(O)NR^{A}R^{B} and R^{A} is H, C₁₋₄ alkyl, or arylalkyl substituted by halo, then R^{B} is other than C₁₋₄ alkyl optionally substituted by COOH, COO(C₁₋₄ alkyl), aryl substituted by halo, or aryloxy substituted by 1 or 2 C₁₋₆ alkyl; and
(c) R³ is other than N-substituted piperidin-3-yl.

2. The compound of claim 1, or pharmaceutically acceptable salt thereof, wherein:
Q is —C(O)NR^{A}R^{B};
R^{A} is H, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl, wherein each of said C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl is optionally substituted by 1, 2, 3, 4 or 5 —W—X—Y-Z; and
R^{B} is cycloalkyl, heterocycloalkyl, cycloalkylalkyl or heterocycloalkylalkyl, each optionally substituted by 1, 2, 3, 4 or 5 —W—X—Y-Z.

3. The compound of claim 1, or pharmaceutically acceptable salt thereof, wherein:
Q is —C(O)NR^{A}R^{B};
R^{A} is H, C₁₋₁₀ alkyl, C₂₋₁₀ alkenyl, C₂₋₁₀ alkynyl, cycloalkyl, heterocycloalkyl, cycloalkylalkyl or heterocycloalkylalkyl, wherein each of said C₁₋₁₀ alkyl, C₂₋₁₀ alkenyl, C₂₋₁₀ alkynyl, cycloalkyl, heterocycloalkyl, cycloalkylalkyl or heterocycloalkylalkyl is optionally substituted by 1, 2, 3, 4 or 5 —W—X—Y-Z; and
R^{B} is cycloalkyl or heterocycloalkyl, each optionally substituted by 1, 2, 3, 4 or 5 —W—X—Y-Z.

4. The compound of claim 1, or pharmaceutically acceptable salt thereof, wherein:
Q is —C(O)NR^{A}R^{B}; and
R^{A} and R^{B} together with the N atom to which they are attached form a 4-20 membered heterocycloalkyl ring optionally substituted by 1, 2, 3, 4 or 5 —W—X—Y-Z.

5. The compound of claim 4, or pharmaceutically acceptable salt thereof, wherein:
R^{A} and R^{B} together with the N atom to which they are attached form a moiety having the formula: 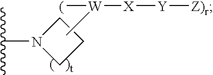
wherein:
r is 0, 1, 2, 3, 4 or 5; and
t is 1, 2, 3, 4 , or 5.

6. The compound of claim 4, or pharmaceutically acceptable salt thereof, wherein:
R^{A} and R^{B} together with the N atom to which they are attached form a moiety having the formula: 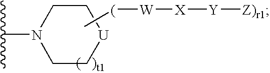
wherein:
r1 is 0, 1, 2 or 3;
t1 is 0 or 1; and
U is CH₂, NH or O.

7. The compound of claim 4, or pharmaceutically acceptable salt thereof, wherein:
R^{A} and R^{B} together with the N atom to which they are attached form a moiety having the formula: 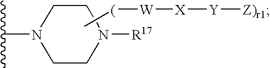
wherein:
r1 is 0, 1, 2 or 3;
R¹⁷ is C(O)R^{b}, C(O)NR^{c}R^{d}, C(O)OR^{a}, C₁₋₆ alkyl, aryl or heteroaryl, wherein each of said C₁₋₆ alkyl, aryl or heteroaryl is optionally substituted by 1, 2 or 3, halo, C₁₋₄ alkyl, C₁₋₄ alkoxy or C₁₋₄ haloalkyl.

8. The compound of claim 4, or pharmaceutically acceptable salt thereof, wherein:
R^{A} and R^{B} together with the N atom to which they are attached form a moiety having the formula: 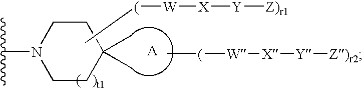
wherein:
ring A is a 3-14 membered cycloalkyl group or a 3-14 membered heterocycloalkyl group;
r1 is 0, 1, 2 or 3; and
r2 is 0, 1, 2, or 3.

9. The compound of claim 8, or pharmaceutically acceptable salt thereof, wherein ring A is a 5-10 membered heterocycloalkyl group.

10. The compound of claim 4, or pharmaceutically acceptable salt thereof, wherein:
R^{A} and R^{B} together with the N atom to which they are attached form a moiety having formula IIa or IIb: 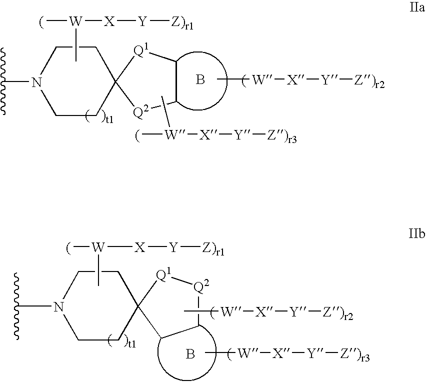
wherein:
Q¹ is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;
Q² is O, S, NH, CH₂, CO, CS, SO, SO₂, OCH₂, SCH₂, NHCH₂, CH₂CH₂, CH═CH, COCH₂, CONH, COO, SOCH₂, SONH, SO₂CH₂, or SO₂NH;
ring B is a fused 5- or 6-membered aryl or fused 5- or 6-membered heteroaryl group;
r1 is 0, 1 or 2;
r2 is 0, 1 or 2;
r3 is 0, 1, or 2; and
the sum of r1, r2 and r3 is 0, 1, 2 or 3.

11. The compound of claim 4, or pharmaceutically acceptable salt thereof, wherein R^{A} and R^{B} together with the N atom to which they are attached form pyrrolidinyl, piperidinyl, piperizinyl, morpholino, 1,2,3,6-tetrahydro-pyridinyl, 3-oxo-piperazinyl, azepanyl or azocanyl, each optionally substituted by 1, 2 or 3 OH, CN, C₁₋₄ alkyl, C₁₋₄ alkoxy, arylalkyl, heterocycloalkyl, aryl, heteroaryl, NR^{c}C(O)R^{d}, NR^{c}C(O)OR^{a}, C(O)R^{b}, C(O)NR^{c}R^{d} or C(O)OR^{a}, wherein each of said aryl or heteroaryl is optionally substituted by 1, 2 or 3 halo, CN, C₁₋₄ alkyl, C₁₋₄ alkoxy or C₁₋₄ haloalkyl.

12. The compound of claim 1, or pharmaceutically acceptable salt thereof, wherein R² is H.

13. The compound of claim 1, or pharmaceutically acceptable salt thereof, wherein R³ is cycloalkyl or heterocycloalkyl, each optionally substituted by 1, 2 or 3 —W′—X′—Y′-Z′.

14. The compound of claim 1, or pharmaceutically acceptable salt thereof, wherein R³ is cycloalkyl or heterocycloalkyl, each optionally substituted by OH.

15. The compound of claim 1, or pharmaceutically acceptable salt thereof, wherein R³ is adamantyl optionally substituted by 1, 2 or 3 —W′—X′—Y′-Z′.

16. The compound of claim 1, or pharmaceutically acceptable salt thereof, wherein R³ is adamantyl optionally substituted by OH.

17. The compound of claim 1, or pharmaceutically acceptable salt thereof, wherein:
R³ is NR^{3a}R^{3b}; and
R^{3a} and R^{3b} together with the N atom to which they are attached form a 4-14 membered heterocycloalkyl group which is optionally substituted by 1, 2 or 3 —W′—X′—Y′-Z′.

18. The compound of claim 1, or pharmaceutically acceptable salt thereof, wherein R³ is 8-azabicyclo[3.2.1]octanyl optionally substituted by 1, 2 or 3 —W′—X′—Y′-Z′.

19. The compound of claim 1, or pharmaceutically acceptable salt thereof, wherein R³ is 8-azabicyclo[3.2.1]octanyl optionally substituted by OH.

20. The compound of claim 1, or pharmaceutically acceptable salt thereof, wherein R⁴, R⁵, R⁶, R⁷, R⁸, R⁹, R¹⁰ and R¹¹ are each H.

21. The compound of claim 1, or pharmaceutically acceptable salt thereof, wherein R¹ is H.

22. The compound of claim 1, or pharmaceutically acceptable salt thereof, wherein R² is H.

23. The compound of claim 1, or pharmaceutically acceptable salt thereof, having Formula III: 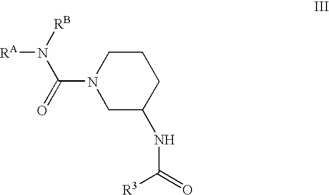
wherein R^{A} and R^{B} together with the N atom to which they are attached form a 4-20 membered heterocycloalkyl ring which is optionally substituted by 1, 2, 3, 4 or 5 —W—X—Y-Z.

24. The compound of claim 23, or pharmaceutically acceptable salt thereof, wherein R³ is cycloalkyl or heterocycloalkyl, each optionally substituted by 1, 2 or 3 —W′—X′—Y′-Z.

25. The compound of claim 1, or pharmaceutically acceptable salt thereof, wherein —W—X—Y-Z is each, independently, OH, CN, C₁₋₄ alkyl, C₁₋₄ alkoxy, arylalkyl, heterocycloalkyl, aryl, heteroaryl, NR^{c}C(O)R^{d}, NR^{c}C(O)OR^{a}, C(O)R^{b}, C(O)NR^{c}R^{d} or C(O)OR^{a}, wherein each of said aryl or heteroaryl is optionally substituted by 1, 2 or 3 halo, CN, C₁₋₄ alkyl, C₁₋₄ alkoxy or C₁₋₄ haloalkyl.

26. The compound of claim 1, or pharmaceutically acceptable salt thereof, wherein —W′—X′—Y′-Z′ is OH.

27. The compound of claim 1, or pharmaceutically acceptable salt thereof, wherein —W″—X″—Y″-Z″ is aryl, C(O)R^{b} or C(O)OR^{a}.

28. A compound selected from:
4-Hydroxy-N-[(3S)-1-(pyrrolidin-1-ylcarbonyl)piperidin-3-yl]adamantane-1-carboxamide;
4-Hydroxy-N-[(3S)-1-(piperidin-1-ylcarbonyl)piperidin-3-yl]adamantane--carboxamide;
4-Hydroxy-N-[(3S)-1-(morpholin-4-ylcarbonyl)piperidin-3-yl]adamantane-1-carboxamide;
(3S)-N-Cyclohexyl-3-{[(4-hydroxy-1-adamantyl)carbonyl]amino}piperidine-1-carboxamide;
4-Hydroxy-N-{(3S)-1-[(4-methoxypiperidin-1-yl)carbonyl]piperidin-3-yl}adamantane-1-carboxamide;
4-Hydroxy-N-{(3S)-1-[(4-methylpiperidin-1-yl)carbonyl]piperidin-3-yl}adamantane-1-carboxamide;
4-Hydroxy-N-{(3S)-1-[(4-phenylpiperidin-1-yl)carbonyl]piperidin-3-yl}adamantane-1-carboxamide;
N-((3S)-1-{[(3R)-3-(Acetylamino)pyrrolidin-1-yl]carbonyl}piperidin-3-yl)-4-hydroxyadamantane-1-carboxamide;
4-Hydroxy-N-((3S)-1-{[(3R)-3-methoxypyrrolidin-1-yl]carbonyl}piperidin-3-yl)adamantane-1-carboxamide;
N-((3S)-1-{[3-(3-Fluorophenyl)pyrrolidin-1-yl]carbonyl}piperidin-3-yl)-4-hydroxyadamantane-1-carboxamide;
N-{(3S)-1-[(4-Cyanopiperidin-1-yl)carbonyl]piperidin-3-yl}-4-hydroxyadamantane-1-carboxamide;
N-((3S)-1-{[(3R)-3-Cyanopyrrolidin-1-yl]carbonyl}piperidin-3-yl)-4-hydroxyadamantane-1-carboxamide;
4-Hydroxy-N-{(3S)-1-[(4-pyridin-4-ylpiperidin-1-yl)carbonyl]piperidin-3-yl}adamantane-1-carboxamide;
4-Hydroxy-N-{(3S)-1-[(4-phenylpiperazin-1-yl)carbonyl]piperidin-3-yl}adamantane-1-carboxamide;
(3-endo)-N-[(3S)-1-(Azepan-1-ylcarbonyl)piperidin-3-yl]-3-hydroxy-8-azabicyclo[3.2.1]octane-8-carboxamide;
(3-endo)-N-[(3S)-1-(Azocan-1-ylcarbonyl)piperidin-3-yl]-3-hydroxy-8-azabicyclo[3.2.1]octane-8-carboxamide;
4-Hydroxy-N-((3S)-1-{[4-(2-methoxyphenyl)piperazin-1-yl]carbonyl}piperidin-3-yl)adamantane-1-carboxamide;
N-((3S)-1-{[4-(2-Ethoxyphenyl)piperazin-1-yl]carbonyl}piperidin-3-yl)-4-hydroxyadamantane-1-carboxamide;
N-((3S)-1-{[4-(2-Fluorophenyl)piperazin-1-yl]carbonyl}piperidin-3-yl)-4-hydroxyadamantane-1-carboxamide;
N-((3S)-1-{[4-(2-Chlorophenyl)piperazin-1-yl]carbonyl}piperidin-3-yl)-4-hydroxyadamantane-1-carboxamide;
4-Hydroxy-N-[(3S)-1-({4-[2-(Trifluoromethyl)phenyl]piperazin-1-yl}carbonyl)piperidin-3-yl]adamantane-1-carboxamide;
4-Hydroxy-N-((3S)-1-{[4-(3-methoxyphenyl)piperazin-1-yl]carbonyl}piperidin-3-yl)adamantane-1-carboxamide;
4-Hydroxy-N-((3S)-1-{[4-(3-methylphenyl)piperazin-1-yl]carbonyl}piperidin-3-yl)adamantane-1-carboxamide;
N-((3S)-1-{[4-(3-Chlorophenyl)piperazin-1-yl]carbonyl}piperidin-3-yl)-4-hydroxyadamantane-1-carboxamide;
4-Hydroxy-N-[(3S)-1-({4-[3-(trifluoromethyl)phenyl]piperazin-1-yl}carbonyl)piperidin-3-yl]adamantane-1-carboxamide;
4-Hydroxy-N-((3S)-1-{[4-(4-methoxyphenyl)piperazin-1-yl]carbonyl}piperidin-3-yl)adamantane-1-carboxamide;
N-((3S)-1-{[4-(4-Fluorophenyl)piperazin-1-yl]carbonyl}piperidin-3-yl)-4-hydroxyadamantane-1-carboxamide;
N-((3S)-1-{[4-(4-Chlorophenyl)piperazin-1-yl]carbonyl}piperidin-3-yl)-4-hydroxyadamantane-1-carboxamide;
N-((3S)-1-{[4-(4-Cyanophenyl)piperazin-1-yl]carbonyl}piperidin-3-yl)-4-hydroxyadamantane-1-carboxamide;
4-Hydroxy-N-((3S)-1-{[3-methyl-4-(3-methylphenyl)piperazin-1-yl]carbonyl}piperidin-3-yl)adamantane-1-carboxamide;
N-((3S)-1-{[4-(2,4-Dimethylphenyl)piperazin-1-yl]carbonyl}piperidin-3-yl)-4-hydroxyadamantane-1-carboxamide;
N-((3S)-1-{[4-(2,5-dimethylphenyl)piperazin-1-yl]carbonyl}piperidin-3-yl)-4-hydroxyadamantane-1-carboxamide;
N-((3S)-1-{[4-(3,4-Dichlorophenyl)piperazin-1-yl]carbonyl}piperidin-3-yl)-4-hydroxyadamantane-1-carboxamide;
N-((3S)-1-{[4-(2,4-Dimethylphenyl)piperazin-1-yl]carbonyl}piperidin-3-yl)-4-hydroxyadamantane-1-carboxamide;
N-((3S)-1-{[4-(5-Chloro-2-methylphenyl)piperazin-1-yl]carbonyl}piperidin-3-yl)-4-hydroxyadamantane-1-carboxamide;
4-Hydroxy-N-((3S)-1-{[4-(2-methylphenyl)piperazin-1-yl]carbonyl}piperidin-3-yl)adamantane-1-carboxamide;
N-((3S)-1-{[4-(2-Cyanophenyl)piperazin-1-yl]carbonyl}piperidin-3-yl)-4-hydroxyadamantane-1-carboxamide;
4-Hydroxy-N-{(3S)-1-[(4-pyridin-4-ylpiperazin-1-yl)carbonyl]piperidin-3-yl}adamantane-1-carboxamide;
4-Hydroxy-N-{(3S)-1-[(4-pyridin-2-ylpiperazin-1-yl)carbonyl]piperidin-3-yl}adamantane-1-carboxamide;
4-Hydroxy-N-{(3S)-1-[(4-pyrimidin-2-ylpiperazin-1-yl)carbonyl]piperidin-3-yl}adamantane-1-carboxamide;
4-Hydroxy-N-{(3S)-1-[(4-pyrazin-2-ylpiperazin-1-yl)carbonyl]piperidin-3-yl}adamantane-1-carboxamide;
N-((3S)-1-{[4-(3,5-Dichloropyridin-4-yl)piperazin-1-yl]carbonyl}piperidin-3-yl)-4-hydroxyadamantane-1-carboxamide;
4-Hydroxy-N-[(3S)-1-({4-[3-(trifluoromethyl)pyridin-2-yl]piperazin-1-yl}carbonyl)piperidin-3-yl]adamantane-1-carboxamide;
N-[(3S)-1-({4-[3-Chloro-5-(trifluoromethyl)pyridin-2-yl]piperazin-1-yl}carbonyl)piperidin-3-yl]-4-hydroxyadamantane-1-carboxamide;
N-{(3S)-1-[(4-Acetylpiperazin-1-yl)carbonyl]piperidin-3-yl}-4-hydroxyadamantane-1-carboxamide;
Ethyl 4-[((3S)-3-{[(4-hydroxy-1-adamantyl)carbonyl]amino}piperidin-1-yl)carbonyl]piperazine-1-carboxylate;
N-((3S)-1-{[4-(2-Furoyl)piperazin-1-yl]carbonyl}piperidin-3-yl)-4-hydroxyadamantane-1-carboxamide;
N-{(3S)-1-[(4-Ethylpiperazin-1-yl)carbonyl]piperidin-3-yl}-4-hydroxyadamantane-1-carboxamide;
N-((3S)-1-{[4-(4-Fluorophenyl)piperidin-1-yl]carbonyl}piperidin-3-yl)-4-hydroxyadamantane-1-carboxamide;
N-{(3S)-1-[(4-Cyano-4-phenylpiperidin-1-yl)carbonyl]piperidin-3-yl}-4-hydroxyadamantane-1-carboxamide;
4-Hydroxy-N-((3S)-1-{[4-(3-methoxyphenyl)piperidin-1-yl]carbonyl}piperidin-3-yl)adamantane-1-carboxamide;
4-Hydroxy-N-{(3S)-1-[(3-oxo-2,8-diazaspiro[4.5]dec-8-yl)carbonyl]piperidin-3-yl}adamantane-1-carboxamide;
4-Hydroxy-N-{(3S)-1-[(2-oxo-1,2-dihydro-1′H-spiro[indole-3,4′-piperidin]-1′-yl)carbonyl]piperidin-3-yl}adamantane-1-carboxamide;
4-Hydroxy-N-{(3S)-1-[(4-phenyl-3,6-dihydropyridin-1(2H)-yl)carbonyl]piperidin-3-yl}adamantane-1-carboxamide;
N-((3S)-1-{[4-(4-Chlorophenyl)-3,6-dihydropyridin-1(2H)-yl]carbonyl}piperidin-3-yl)-4-hydroxyadamantane-1-carboxamide;
4-Hydroxy-N-((3S)-1-{[(1R)-3-oxo-1′H,3H-spiro[2-benzofuran-1,3′-pyrrolidin]-1′-yl]carbonyl}piperidin-3-yl)adamantane-1-carboxamide;
4-Hydroxy-N-((3S)-1-{[(1R)-3-oxo-1′H,3H-spiro[furo[3,4-c]pyridine-1,3′-pyrrolidin]-1′-yl]carbonyl}piperidin-3-yl)adamantane-1-carboxamide;
4-Hydroxy-N-{(3S)-1-[(4-hydroxy-4-phenylpiperidin-1-yl)carbonyl]piperidin-3-yl}adamantane-1-carboxamide;
4-Hydroxy-N-[(3S)-1-(1′H,3H-spiro[2-benzofuran-1,4′-piperidin]-1′-ylcarbonyl)piperidin-3-yl]adamantane-1-carboxamide;
4-Hydroxy-N-{(3S)-1-[(4-oxo-1-phenyl-1,3,8-triazaspiro[4.5]dec-8-yl)carbonyl]piperidin-3-yl}adamantane-1-carboxamide;
N-[(3S)-1-(1,4′-Bipiperidin-1′-ylcarbonyl)piperidin-3-yl]-4-hydroxyadamantane-1-carboxamide;
4-Hydroxy-N-((3S)-1-{[4-(2-oxo-2,3-dihydro-1H-benzimidazol-1-yl)piperidin-1-yl]carbonyl}piperidin-3-yl)adamantane-1-carboxamide;
4-Hydroxy-N-((3S)-1-{[4-(1H-indol-1-yl)piperidin-1-yl]carbonyl}piperidin-3-yl)adamantane-1-carboxamide;
tert-Butyl {1-[((3S)-3-{[(4-hydroxy-1-adamantyl)carbonyl]amino}piperidin-1-yl)carbonyl]piperidin-4-yl}carbamate;
4-Hydroxy-N-[(3S)-1-({4-[phenyl(propionyl)amino]piperidin-1-yl}carbonyl)piperidin-3-yl]adamantane-1-carboxamide;
N-{(3S)-1-[(4-Benzylpiperidin-1-yl)carbonyl]piperidin-3-yl}-4-hydroxyadamantane-1-carboxamide;
N-{(3S)-1-[(4-Benzyl-4-hydroxypiperidin-1-yl)carbonyl]piperidin-3-yl}-4-hydroxyadamantane-1-carboxamide;
tert-Butyl 8-[((3S)-3-{[(4-hydroxy-1-adamantyl)carbonyl]amino}piperidin-1-yl)carbonyl]-2,8-diazaspiro[4.5]decane-2-carboxylate;
tert-Butyl 4-[((3S)-3-{[(4-hydroxy-1-adamantyl)carbonyl]amino}piperidin-1-yl)carbonyl]piperazine-1-carboxylate;
(3-endo)-3-Hydroxy-N-[(3S)-1-({4-[3-(trifluoromethyl)pyridin-2-yl]piperazin-1-yl}carbonyl)piperidin-3-yl]-8-azabicyclo[3.2.1]octane-8-carboxamide;
(3-endo)-3-Hydroxy-N-((3S)-1-{[4-(2-methoxyphenyl)piperazin-1-yl]carbonyl}piperidin-3-yl)-8-azabicyclo[3.2.1]octane-8-carboxamide;
(3-endo)-N-((3S)-1-{[4-(2-Ethoxyphenyl)piperazin-1-yl]carbonyl}piperidin-3-yl)-3-hydroxy-8-azabicyclo[3.2.1]octane-8-carboxamide;
(3-endo)-3-Hydroxy-N-((3S)-1-{[4-(4-methoxyphenyl)piperazin-1-yl]carbonyl}piperidin-3-yl)-8-azabicyclo[3.2.1]octane-8-carboxamide;
(3-endo)-3-Hydroxy-N-{(3S)-1-[(4-pyrazin-2-ylpiperazin-1-yl)carbonyl]piperidin-3-yl}-8-azabicyclo[3.2.1]octane-8-carboxamide;
4-Hydroxy-N-{(3S)-1-[(3-oxopiperazin-1-yl)carbonyl]piperidin-3-yl}adamantane-1-carboxamide;
4-Hydroxy-N-{(3S)-1-[(3-oxo-1′H,3H-spiro[2-benzofuran-1,4′-piperidin]-1′-yl)carbonyl]piperidin-3-yl}adamantane-1-carboxamide;
4-Hydroxy-N-((3S)-1-{[(3R,4R)-3-hydroxy-4-phenylpiperidin-1-yl]carbonyl}piperidin-3-yl)adamantane-1-carboxamide;
1-[((3S)-3-{[(4-Hydroxy-1-adamantyl)carbonyl]amino}piperidin-1-yl)carbonyl]-N,N-dimethylpiperidine-4-carboxamide;
N-((3S)-1-{[2-(Cyclopentylcarbonyl)-2,8-diazaspiro[4.5]dec-8-yl]carbonyl}piperidin-3-yl)-4-hydroxyadamantane-1-carboxamide;
Methyl 8-[((3S)-3-{[(4-hydroxy-1-adamantyl)carbonyl]amino}piperidin-1-yl)carbonyl]-2,8-diazaspiro[4.5]decane-2-carboxylate;
N-((3S)-1-{[4-(Cyclopentylcarbonyl)piperazin-1-yl]carbonyl}piperidin-3-yl)-4-hydroxyadamantane-1-carboxamide;
Methyl 4-[((3S)-3-{[(4-hydroxy-1-adamantyl)carbonyl]amino}piperidin-1-yl)carbonyl]piperazine-1-carboxylate;
Ethyl 4-[((3S)-3-{[(4-hydroxy-1-adamantyl)carbonyl]amino}piperidin-1-yl)carbonyl]piperazine-1-carboxylate;
N-[(3S)-1-({4-[(Cyclopentylcarbonyl)amino]piperidin-1-yl}carbonyl)piperidin-3-yl]4-hydroxyadamantane-1-carboxamide;
Methyl {1-[((3S)-3-{[(4-hydroxy-1-adamantyl)carbonyl]amino}piperidin-1-yl)carbonyl]piperidin-4-yl}carbamate;
N-((3S)-1-{[4-(Benzoylamino)piperidin-1-yl]carbonyl}piperidin-3-yl)-4-hydroxyadamantane-1-carboxamide; and
4-Hydroxy-N-[(3S)-1-(1′H-spiro[chromene-2,4′-piperidin]-1′-ylcarbonyl)piperidin-3-yl]adamantane-1-carboxamide,
or a pharmaceutically acceptable salt thereof.

29. A composition comprising a compound of claim 1, or pharmaceutically acceptable salt thereof, and a pharmaceutically acceptable carrier.

30. A method of modulating 11βHSD1 or MR comprising contacting said 11βHSD1 or MR with a compound of a compound of Formula I: 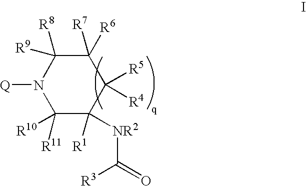
or a pharmaceutically acceptable salt or prodrug thereof, wherein:
Q is —SO₂-Cy, —C(O)O-Cy or —C(O)NR^{A}R^{B};
Cy is cycloalkyl or heterocycloalkyl, each optionally substituted by 1, 2, 3, 4 or 5 —W—X—Y-Z;
R^{A} and R^{B} are independently selected from H, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl, and heterocycloalkylalkyl, wherein said C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 —W—X—Y-Z;
or R^{A} and R^{B} together with the N atom to which they are attached form a 4-20 membered heterocycloalkyl ring optionally substituted with 1, 2, 3, 4 or 5 —W—X—Y-Z;
R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;
R² is H, C₁₋₆ alkyl, arylalkyl, heteroarylalkyl, cycloalkyl, cycloalkylalkyl, heterocycloalkyl or heterocycloalkylalkyl, wherein said C₁₋₆ alkyl, arylalkyl, heteroarylalkyl, cycloalkyl, cycloalkylalkyl, heterocycloalkyl or heterocycloalkylalkyl is optionally substituted by 1, 2 or 3 R¹⁴;
R³ is H, NR^{3a}R^{3b}, C₁₋₆ alkyl, aryl, cycloalkyl, heteroaryl, or heterocycloalkyl, wherein said C₁₋₆ alkyl, aryl, cycloalkyl, heteroaryl, or heterocycloalkyl is optionally substituted by 1, 2 or 3 —W′—X′—Y′-Z′;
R^{3a} and R^{3b} are independently selected from H, C₁₋₆ alkyl, aryl, cycloalkyl, heteroaryl, and heterocycloalkyl, wherein said C₁₋₆ alkyl, aryl, cycloalkyl, heteroaryl, or heterocycloalkyl is optionally substituted by 1, 2 or 3 —W′—X′—Y′-Z′;
or R^{3a} and R^{3b} together with the N atom to which they are attached form a 4-14 membered heterocycloalkyl group which is optionally substituted by 1, 2 or 3 —W′—X′—Y′-Z′;
R⁴, R⁵, R⁶, R⁷, R⁸, R⁹, R¹⁰ and R¹¹ are independently selected from H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl is optionally substituted by 1, 2 or 3 R¹⁴;
or R² and R³ together with the nitrogen and carbon atoms to which they are attached form a 3-14 membered heterocycloalkyl group which is optionally substituted by 1, 2 or 3 R¹⁴;
or R⁴ and R⁵ together with the carbon atom to which they are attached form a 3-14 membered cycloalkyl or 3-14 membered heterocycloalkyl group which is optionally substituted by 1, 2 or 3 R¹⁴;
or R⁶ and R⁷ together with the carbon atom to which they are attached form a 3-14 membered cycloalkyl or 3-14 membered heterocycloalkyl group which is optionally substituted by 1, 2 or 3 R¹⁴;
or R⁸ and R⁹ together with the carbon atom to which they are attached form a 3-14 membered cycloalkyl or 3-14 membered heterocycloalkyl group which is optionally substituted by 1, 2 or 3 R¹⁴;
or R¹⁰ and R¹¹ together with the carbon atom to which they are attached form a 3-14 membered cycloalkyl or 3-14 membered heterocycloalkyl group which is optionally substituted by 1, 2 or 3 R¹⁴;
or R⁴ and R⁶ together with the carbon atoms to which they are attached form a 3-7 membered fused cycloalkyl group or 3-7 membered fused heterocycloalkyl group which is optionally substituted by 1, 2 or 3 R¹⁴;
or R⁶ and R⁸ together with the carbon atoms to which they are attached form a 3-7 membered fused cycloalkyl group or 3-7 membered fused heterocycloalkyl group which is optionally substituted by 1, 2 or 3 R¹⁴;
R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of said C₁₋₆ alkylenyl, C₂₋₆ alkenylenyl, or C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino, and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl, and heterocycloalkyl, wherein each of said C₁₋₆ alkylenyl, C₂₋₆ alkenylenyl, C₂₋₆ alkynylenyl, cycloalkyl, heteroaryl, and heterocycloalkyl is optionally substituted by one or more substituents independently selected from halo, CN, NO₂, OH, C₁₋₄ alkoxy, C₁₋₄ haloalkoxy, amino, C₁₋₄ alkylamino, and C₂₋₈ dialkylamino;
Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein each of said C₁₋₆ alkylenyl, C₂₋₆ alkenylenyl, and C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino, and C₂₋₈ dialkylamino;
Z, Z′ and Z″ are independently selected from H, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, pentahalosulfanyl, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl, and heterocycloalkyl, wherein each of said C₁₋₆ alkyl, C₂₋₆ alkenyl, C₂₋₆ alkynyl, aryl, cycloalkyl, heteroaryl or heterocycloalkyl is optionally substituted by 1, 2 or 3 substituents independently selected from halo, C₁₋₆ alkyl, C₂₋₆ alkenyl, C₂₋₆ alkynyl, C₁₋₄ haloalkyl, aryl, cycloalkyl, heteroaryl, heterocycloalkyl, pentahalosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkyl or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{e} and R^{f} together with the N atom to which they are attached form a 4-, 5-, 6- or 7-membered heterocycloalkyl group; and
q is 1 or 2.

31. The method of claim 30 wherein said modulating is inhibiting.

32. A method of treating a disease in a patient, wherein said disease is associated with expression or activity of 11βHSD1 or MR, comprising administering to said patient a therapeutically effective amount of a compound of Formula I: 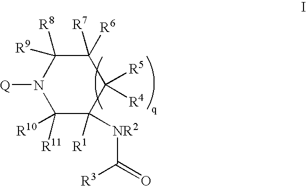
or a pharmaceutically acceptable salt or prodrug thereof, wherein:
Q is —SO₂-Cy, —C(O)O-Cy or —C(O)NR^{A}R^{B};
Cy is cycloalkyl or heterocycloalkyl, each optionally substituted by 1, 2, 3, 4 or 5 —W—X—Y-Z;
R^{A} and R^{B} are independently selected from H, C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl, and heterocycloalkylalkyl, wherein said C₁₋₁₀ alkyl, C₂₋₁₀ alkenyl, C₂₋₁₀ alkynyl, cycloalkyl, heterocycloalkyl, arylalkyl, heteroarylalkyl, cycloalkylalkyl or heterocycloalkylalkyl are each optionally substituted with 1, 2, 3, 4 or 5 —W—X—Y-Z;
or R^{A} and R^{B} together with the N atom to which they are attached form a 4-20 membered heterocycloalkyl ring optionally substituted with 1, 2, 3, 4 or 5 —W—X—Y-Z;
R¹ is H, C(O)OR^{b′}, S(O)R^{a′}, S(O)NR^{c′}R^{d′}, S(O)₂R^{a′}, S(O)₂NR^{c′}R^{d′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl is optionally substituted by 1, 2 or 3 R¹⁴;
R² is H, C₁₋₆ alkyl, arylalkyl, heteroarylalkyl, cycloalkyl, cycloalkylalkyl, heterocycloalkyl or heterocycloalkylalkyl, wherein said C₁₋₆ alkyl, arylalkyl, heteroarylalkyl, cycloalkyl, cycloalkylalkyl, heterocycloalkyl or heterocycloalkylalkyl is optionally substituted by 1, 2 or 3 R¹⁴;
R³ is H, NR^{3a}R^{3b}, C₁₋₆ alkyl, aryl, cycloalkyl, heteroaryl, or heterocycloalkyl, wherein said C₁₋₆ alkyl, aryl, cycloalkyl, heteroaryl, or heterocycloalkyl is optionally substituted by 1, 2 or 3 —W′—X′—Y′-Z′;
R^{3a} and R^{3b} are independently selected from H, C₁₋₆ alkyl, aryl, cycloalkyl, heteroaryl, and heterocycloalkyl, wherein said C₁₋₆ alkyl, aryl, cycloalkyl, heteroaryl, or heterocycloalkyl is optionally substituted by 1, 2 or 3 —W′—X′—Y′-Z′;
or R^{3a} and R^{3b} together with the N atom to which they are attached form a 4-14 membered heterocycloalkyl group which is optionally substituted by 1, 2 or 3 —W′—X′—Y′-Z′;
R⁴, R⁵, R⁶, R⁷, R⁸, R⁹, R¹⁰ and R¹¹ are independently selected from H, OC(O)R^{a′}, OC(O)OR^{b′}, C(O)OR^{b′}, OC(O)NR^{c′}R^{d′}, NR^{c′}R^{d′}, NR^{c′}C(O)R^{a′}, NR^{c′}C(O)OR^{b′}, S(O)R^{a′}, S(O)NR^{c′}R^{d′}, S(O)₂R^{a′}, S(O)₂NR^{c′}R^{d′}, SR^{b′}, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, and heterocycloalkylalkyl, wherein said C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl, or heterocycloalkylalkyl is optionally substituted by 1, 2 or 3 R¹⁴;
or R² and R³ together with the nitrogen and carbon atoms to which they are attached form a 3-14 membered heterocycloalkyl group which is optionally substituted by 1, 2 or 3 R¹⁴;
or R⁴ and R⁵ together with the carbon atom to which they are attached form a 3-14 membered cycloalkyl or 3-14 membered heterocycloalkyl group which is optionally substituted by 1, 2 or 3 R¹⁴;
or R⁶ and R⁷ together with the carbon atom to which they are attached form a 3-14 membered cycloalkyl or 3-14 membered heterocycloalkyl group which is optionally substituted by 1, 2 or 3 R¹⁴;
or R⁸ and R⁹ together with the carbon atom to which they are attached form a 3-14 membered cycloalkyl or 3-14 membered heterocycloalkyl group which is optionally substituted by 1, 2 or 3 R¹⁴;
or R¹⁰ and R¹¹ together with the carbon atom to which they are attached form a 3-14 membered cycloalkyl or 3-14 membered heterocycloalkyl group which is optionally substituted by 1, 2 or 3 R¹⁴;
or R⁴ and R⁶ together with the carbon atoms to which they are attached form a 3-7 membered fused cycloalkyl group or 3-7 membered fused heterocycloalkyl group which is optionally substituted by 1, 2 or 3 R¹⁴;
or R⁶ and R⁸ together with the carbon atoms to which they are attached form a 3-7 membered fused cycloalkyl group or 3-7 membered fused heterocycloalkyl group which is optionally substituted by 1, 2 or 3 R¹⁴;
R¹⁴ is halo, C₁₋₄ alkyl, C₁₋₄ haloalkyl, aryl, cycloalkyl, heteroaryl, heterocycloalkyl, CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{d′}, NR^{c′}C(O)OR^{a′}, S(O)R^{b′}, S(O)NR^{c′}R^{d′}, S(O)₂R^{b′}, or S(O)₂NR^{c′}R^{d′};
W, W′ and W″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, and NR^{e}CONR^{f}, wherein each of said C₁₋₆ alkylenyl, C₂₋₆ alkenylenyl, or C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino, and C₂₋₈ dialkylamino;
X, X′ and X″ are independently selected from absent, C₁₋₆ alkylenyl, C₂₋₆ alkenylenyl, C₂₋₆ alkynylenyl, aryl, cycloalkyl, heteroaryl, and heterocycloalkyl, wherein each of said C₁₋₆ alkylenyl, C₂₋₆ alkenylenyl, C₂₋₆ alkynylenyl, cycloalkyl, heteroaryl, and heterocycloalkyl is optionally substituted by one or more substituents independently selected from halo, CN, NO₂, OH, C₁₋₄ alkoxy, C₁₋₄ haloalkoxy, amino, C₁₋₄ alkylamino, and C₂₋₈ dialkylamino;
Y, Y′ and Y″ are independently selected from absent, C₁₋₆ alkylenyl, C₂₋₆ alkenylenyl, C₂₋₆ alkynylenyl, O, S, NR^{e}, CO, COO, CONR^{e}, SO, SO₂, SONR^{e}, or NR^{e}CONR^{f}, wherein each of said C₁₋₆ alkylenyl, C₂₋₆ alkenylenyl, and C₂₋₆ alkynylenyl is optionally substituted by 1, 2 or 3 substituents independently selected from halo, OH, C₁₋₄ alkoxy, C₁₋₄ haloalkoxy, amino, C₁₋₄ alkylamino, and C₂₋₈ dialkylamino;
Z, Z′ and Z″ are independently selected from H, halo, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, S(O)₂NR^{c}R^{d}, C₁₋₄ alkoxy, C₁₋₄ haloalkoxy, pentahalosulfanyl, amino, C₁₋₄ alkylamino, C₂₋₈ dialkylamino, C₁₋₆ alkyl, C₂₋₆ alkenyl, C₂₋₆ alkynyl, aryl, cycloalkyl, heteroaryl, and heterocycloalkyl, wherein each of said C₁₋₆ alkyl, C₂₋₆ alkenyl, C₂₋₆ alkynyl, aryl, cycloalkyl, heteroaryl or heterocycloalkyl is optionally substituted by 1, 2 or 3 substituents independently selected from halo, C₁₋₆ alkyl, C₂₋₆ alkenyl, C₂₋₆ alkynyl, C₁₋₄ haloalkyl, aryl, cycloalkyl, heteroaryl, heterocycloalkyl, pentahalosulfanyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{d}, NR^{c}C(O)OR^{a}, S(O)R^{b}, S(O)NR^{c}R^{d}, S(O)₂R^{b}, and S(O)₂NR^{c}R^{d};
wherein two —W—X—Y-Z attached to the same atom optionally form a 3-14 membered cycloalkyl or 3-14 membered heterocycloalkyl group optionally substituted by 1, 2 or 3 —W″—X″—Y″-Z″;
wherein two —W′—X′—Y′-Z′ attached to the same atom optionally form a 3-14 membered cycloalkyl or 3-14 membered heterocycloalkyl group optionally substituted by 1, 2 or 3 —W″—X″—Y″-Z″;
wherein —W—X—Y-Z is other than H;
wherein —W′—X′—Y′-Z′ is other than H;
wherein —W″—X″—Y″-Z″ is other than H;
R^{a} and R^{a′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, aryl, arylalkyl, heteroaryl, heteroarylalkyl, cycloalkyl or heterocycloalkyl;
R^{b} and R^{b′} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
R^{c} and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c} and R^{d} together with the N atom to which they are attached form a 4-, 5-, 6- or 7-membered heterocycloalkyl group;
R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{c′} and R^{d′} together with the N atom to which they are attached form a 4-, 5-, 6- or 7-membered heterocycloalkyl group;
R^{e} and R^{f} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted with H, OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{e} and R^{f} together with the N atom to which they are attached form a 4-, 5-, 6- or 7-membered heterocycloalkyl group; and
q is 1 or 2.

33. The method of claim 32 wherein said disease is obesity, diabetes, glucose intolerance, insulin resistance, hyperglycemia, atherosclerosis, hypertension, hyperlipidemia, cognitive impairment, dementia, depression, glaucoma, cardiovascular disorders, osteoporosis, inflammation, metabolic syndrome, coronary heart disease, type 2 diabetes, hypercortisolemia, androgen excess, or polycystic ovary syndrome (PCOS).
